SEQUENCE LISTING (7 sequences)

SEQ ID NO: 1 - protein, 9 aa, Homo sapiens
YLEPGPVTA

SEQ ID NO: 2 - protein, 9 aa, Homo sapiens
KTWGQYWQV

SEQ ID NO: 3 - protein, 9 aa, Homo sapiens
YMDGTMSQV

SEQ ID NO: 4 - protein, 16 aa, Homo sapiens
WWRQLYPEWTEAQRLD

SEQ ID NO: 5 - protein, 15 aa, Homo sapiens
DYSYLQDSDPDSFQD

SEQ ID NO: 6 - protein, 9 aa, Homo sapiens
ALLAVGATK

SEQ ID NO: 7 - protein, 9 aa, Homo sapiens
SLFRAVITK

(19) United States
(12) Patent Application Publication
(10) Pub. No.: US 2012/0076827 A1
(43) Pub. Date: Mar. 29, 2012

(54) Compositions and methods for generating an immune response in a subject

(75) Inventors: Ingrid Jolanda Monique de Vries, Nijmegen (NL); Carl Gustav Figdor, Hertogenbosch (NL); Daniel Benitez Ribas, Barcelona (ES)

(73) Assignee: Stichting Katholieke Universiteit Radboud University Nijmegen Medical Centre, Nijmegen (NL)

(21) Appl. No.: 12/657,612

(22) Filed: Jan. 22, 2010

Related U.S. Application Data
(63) Continuation of application No. PCT/EP2008/006182, filed on Jul. 24, 2008.
(60) Provisional application No. 60/961,861, filed on Jul. 24, 2007.

(51) Int. Cl. A61K 39/00; A61P 35/00; A61P 37/04; C12N 5/0784

(52) U.S. Cl. 424/277.1; 435/375; 435/325; 424/184.1

(57) ABSTRACT

The invention relates to a method for providing an activated antigen-presenting cell or a composition that comprises at least one activated antigen-presenting cell, which method at last comprises the steps of providing a composition that comprises at least one antigen-presenting cell and contacting said composition with a vaccine. Suitably, the at least one dendritic cell is brought into a state in which it is capable of stimulating T-cells and/or a T-cell mediated response.

The present invention relates to compositions and methods that can be used to generate an immune response in a subject.

In particular, the present invention relates to compositions and methods that can be used to generate an immune response in a subject against one or more predetermined antigens.

The invention also relates to methods for preparing such compositions, as well as to the use of such compositions in (methods for) generating an immune response in a subject.

As will become clear from the further description herein, the compositions used in the present invention comprise activated (as defined herein) antigen-presenting cells (such as dendritic cells) that have been loaded (as defined herein) with the one or more predetermined antigens. As will also become clear from the further description herein, the activating and loading of the antigen-presenting cells may be performed in vitro (such as ex vivo) or in vivo (i.e. in the body of the subject in which the immune response is to be raised). In both these aspects, the invention also provides means and materials (for example biological materials, proteins or polypeptides, or other chemical entities) for performing the methods described herein, which may also be in the form of suitable compositions (as described herein) or a kit-of-parts (also as described herein).

In the methods described herein, the “antigen(s)” may also be cellular antigens, by which is generally meant herein one or more antigens or antigenic determinants that are expressed by or otherwise present in or on cells or tissues against which the immune response is to be raised. These cells or tissues will usually be present in the body of the subject in which the immune response is to be raised, for example to either kill the cells or destroy the tissues and/or to stop, reduce or reverse the (further) proliferation or growth of the cells or tissues (i.e. where it is desired to kill the cell, remove the tissue or prevent or reduce the (further) proliferation or growth of cells or tissue, such as in the case of tumor cells or tumors). As will be further described herein, in such a case, the antigen can for example be any suitable antigen or antigenic determinant that is derived from and/or expressed by the cell or tissue, but may for example also be any suitable fraction (such as, without limitation, a membrane fraction), extract or lysate that has derived from the cells or tissue (or from a similar cell or tissue, such as a tumor cell line), such as, without limitation tumor lysates, tumor cell line lysates, tumor-derived RNA or (other) suitable cell fractions or cell extracts.

Accordingly, it should be understood that all of the foregoing antigens are included in the term “predetermined antigen” as used herein in its broadest sense, even if such an antigen is not fully characterised in the sense that it is has not been fully defined (i.e. in advance or subsequently) against which specific protein, epitope or antigen(ic determinant) present in the predetermined antigen the immune response is raised. For example, when an immune response is raised using a cell fragment, extract or lysate, this cell fragment, extract or lysate is (used as) the “predetermined antigen” as defined herein, even if it is not fully defined or characterised (in advance or subsequently) against which specific protein or antigen(ic determinant) contained or present within said fragment, extract or lysate the immune response obtained is directed. Based on the disclosure herein, it will also be clear to the skilled person that, in the methods of the invention, such a cell fragment, extract or lysate (for example of a tumor cell or tumor cell line) can be used to raise an effective immune response (that is also of practical use in the invention and more generally in the fields of therapy, imaging or diagnosis, for example for the immunotherapy of cancer, as further described herein), even if it is not fully defined or characterised against which specific protein or antigen the immune response is directed. In fact, it is one of the practical advantages of the present invention that such a cell fragment, extract or lysate (for example of a tumor cell or tumor cell line) can be used in the methods described herein to raise an effective immune response against a certain cell or type of cells (for example, against tumor cells), without it being required that a specific protein or antigen(ic determinant) present on said cell or type of cells is identified and characterised in advance, and subsequently isolated and used to raise an immune response (although the use of such a protein or antigen(ic) determinant is also included in the present invention).

Thus, in further aspects, the invention relates to the activated and loaded antigen-presenting cells that can be obtained using the methods described herein, to compositions comprising such activated and loaded antigen-presenting cells, to uses of such activated and loaded antigen-presenting cells and compositions, to methods of treatment involving the use of such activated and loaded antigen-presenting cells and of such compositions, as well as to methods for preparing such activated and loaded antigen-presenting cells and such compositions. The “antigen-presenting cells” may be any suitable antigen presenting cells (as further defined herein), and may in particular be dendritic cells.

In a specific, but non-limiting aspect, the invention relates to methods for immunotherapy in a subject that involve the use of such activated and loaded antigen-presenting cells and/or of such compositions, as well as to activated and loaded antigen-presenting cells or compositions for use in methods of immunotherapy. For example, as further described herein, the methods described herein can be used to provide activated antigen-presenting cells that have been loaded with one or more tumor-derived antigens, and such activated and loaded antigen-presenting cells (or compositions comprising the same) can be used in the immunotherapy of cancer. Again, the “antigen-presenting cells” may be any suitable antigen presenting cells (as further defined herein), and may in particular be dendritic cells.

The invention also relates to methods for activating (as defined herein) antigen-presenting cells so as to provide activated antigen-presenting cells that can be loaded (as defined herein) with one or more antigens in order to provide activated and loaded antigen-presenting cells. The invention also relates to the activated antigen-presenting cells that can be obtained (or have been obtained) using the methods described herein, and to compositions comprising the same.

In another aspect, the invention also provides compounds, constructs or complexes that can be used to activate antigen-presenting cells, that can be used in the methods described herein, and/or that can be administered to a subject (for example systemically or in or near the site where the immune response is to be raised, such as in or in the immediate vicinity of a tumour to be treated) in order to activate at least one antigen-presenting cell (such as a dendritic cell) in the body of said subject, and optionally also to raise an immune response in said subject against one or more desired antigens.

As further described herein, such a compound, construct or complex may generally comprise:
(i) a first moiety that is capable of targeting the compound, construct or complex towards the antigen-presenting cell(s) to be activated (either in vitro, ex vivo or in vivo, i.e. in the body of a subject to be treated). This first moiety may for example be an antibody or antibody fragment directed against the antigen-presenting cell, as further described herein; and in addition one or both of:
(ii) an antigenic compound (i.e. for activating the antigen-presenting cell(s), as further described herein); and/or
(iii) the desired predetermined antigen or antigens (as defined herein) against which the immune response is to be raised. For example, when an immune response is to be raised against a tumor cell, this may be any suitable material or antigen that is derived from said tumor cell (or from an equivalent or similar tumor cell or cell tumor line), such as cellular antigens (as described herein), proteins, polypeptides, or RNA.

As further described herein, such a compound, complex or construct may be targeted towards (e.g. directed against) any suitable or desired “antigen-presenting cells” (as described herein), and may in particular be targeted towards dendritic cells.

As also further described herein, the invention further relates to compositions comprising such a compound, complex or construct (which compositions may in particular be pharmaceutical compositions, as described herein); to kits comprising such a compound, complex, construct or composition; and to applications and methods for using such a compound, complex, construct, composition or kit (for example in immunotherapy, such as the immunotherapy of cancer); all of which may be as further described herein.

Further aspects, embodiments, uses, applications and advantages of the invention will become clear from the further description herein.

The earliest host response to pathogens is the innate immune response, in which dendritic cells (DCs) play a pivotal role. DC's are the most potent antigen presenting cells (APC) of the immune system. Upon infection or inflammation, immature DC are activated and differentiate into mature DC that instruct and activate B and T lymphocytes, the mediators of adaptive cimmunity. As further described herein, DC's can sense pathogens through pathogen-recognition receptors, of which the Toll-like receptors or “TLRs” are a subclass.

Generally, DC's in the blood can be subdivided into two major populations, namely CD11c+ DC's (which are thought to be myeloid-derived and therefore also known as “myeloid DC's” or “mDC's”) and CD11-DC's (which are also called “plasmacytoid-derived DC's” or “pDC's”). Reference is for example made to Gibson et al., Cellular immunology, 218, (2002), 74-86; and to Liu, Cell 106:259-262 (2001).

pDC's are also considered to be precursors of DC's, since pDC's need to be (further) differentiated in order to be capable of stimulating T-cells (e.g. via upregulation of CD80 and CD86). Reference is again made to Gibson et al., as well as to for example Soumelis and Liu, Eur. J. Immunol., 2006, 36.

It is also known that DC's can (further) stimulate T-cells and immune responses. through the production and secretion of cytokines such as, amongst others IL-12 (in the case of mDC's) and interferons such as Type I IFN's (IFN-alpha/beta) (in the case of pDC's). Reference is again made to Gibson et al., to Soumelis and Liu., to Barchet et al., Semin Immunol. August 2005; 17(4): 253-61, and to Mansour Haeryfar, Trends in Immunology, 26, Jun. 2005, 311-317.

In addition, DC's can also stimulate B-cell mediated immune responses. For example, plasmacytoid DCs (pDCs) have the ability to link innate and adaptive immune responses by secretion of type I IFN and increasing costimulatory- and antigen presenting molecules, respectively. DCs expressing these molecules can stimulate antigen-specific T cells, which then can provide help to B cells to produce protective antibodies that generally determine the efficacy of the response of the immune system to the pathogen or antigen.

It is also known that pDC's contribute to innate antiviral and bacterial immune responses by producing type I interferon. The transition of pDCs from plasmacytoid to dendritic morphology and function coincides with their cessation of massive type I IFN production, which can by achieved by viral or bacterial activation leading to the upregulation of costimulatory markers (see for example Soumelis and Liu, supra).

It has further been described that human pDCs are very potent inducers of allogeneic T cell responses and capable of priming specific CD4+ and CD8+ lymphocytes against different types of viruses or tumor antigens (see for example Salio et al., Eur J Immunol. 2003 April; 33(4):1052-62 and Fonteneau et al., Blood. 2003 May 1; 101(9):3520-6).

In vitro, because DC's are the major type I IFN producer and have a high capacity to (cross-)present antigen, activated pDCs are able to expand specific CTLs for tumor antigens. In addition, synergistic interaction between pDCs and mDCs generate Ag-specific antitumor immune responses in mouse models.

DC's may also be cultured (i.e. in vitro/ex vivo) from suitable progenitor cells or precursor cells such as monocytes or CD34+ cells. Reference is for example made to Feuerstein et al., Journal of Immunological Methods, 245 (2000), 15-29 and to the further references mentioned on page 16 thereof. These DC's can for example also give rise to a population of cells known as Langerhans cells.

It is known, for example from the prior art cited herein (see for example Feuerstein et al. and the review by Tuyaerts et al., both cited herein), that dendritic cells (also referred to herein as “DC's”) can be loaded (as defined herein) with one or more predetermined antigens, and that dendritic cells that have been loaded with one or more antigens (herein also referred to as “loaded dendritic cells” or “loaded DC's”) can be used to generate an immune response against said antigen(s) in a subject. For example, for this purpose, dendritic cells (or suitable precursors thereof, such as pDC's —as described herein - or suitable monocytes or cells derived from precursor CD34+ cells)) can be harvested from a patient, loaded ex vivo with the antigen(s) by suitably contacting the activated (as defined herein) dendritic cells with the one or more antigens, upon which the antigens bind to the dendritic cells (and/or are taken up by the dendritic cells) and are (subsequently) loaded onto the MHC complex that is present on the surface of the DC's). When such loaded dendritic cells are subsequently administered to a subject, the loaded dendritic cells present the antigen(s) to effector lymphocytes (CD4+ T cells, CD8+ T cells, and to B cells also) and so are capable of triggering a specific cytotoxic response against the antigen(s); and in particular of stimulating killer T-cells so as to induce a T-cell mediated immune response, and/or of stimulating a B-cell mediated antibody response. Thus, by suitably choosing the antigen(s) used in such “dendritic cell vaccination” techniques, such an immune response can be used to obtain a desired therapeutic and/or prophylactic effect in a subject.

One specific use of DC-vaccines is in methods for cancer immunotherapy. In these methods, tumor-derived antigens are loaded onto (and/or into) the dendritic cells, upon which the dendritic cells are used to target the immune system to these antigens (i.e. by administering the loaded DC's to a subject to be treated). The loaded DC's thus obtained can be used to initiate an immune response against the tumor, but also induce “memory' and can break immunological tolerance against the tumor. Reference is again made to the further prior art cited herein, such as the review by Tuyaerts et al.

It has been shown that pDC are capable of inducing strong human anti-tumor immune responses in-vitro. Also, pDCs in mouse models have been proposed to induce and expand tumor-specific cytotoxic T-cells (see for example Rothenfusser, Blood. 2004 Mar. 15; 103(6):2162-9, Salio et al., supra and Fonteneau et al., supra).

The use of DC-based vaccines based on mDC's and CD34-derived DC's is also being explored in clinical trials, predominantly in cancer patients. In these trials, different subpopulations of antigen presenting cells have been used as vaccines to boost the immune system against malignant tumors in patients with cancer (see for example Banchereau, Dev Biol (Basel). 2004; 116:147-56; and Nestle, Curr Opin Immunol. 2005 April; 17(2):163-9.)

The currently used DC-based vaccines consist of antigen-loaded autologous monocyte-derived DCs that are administrated to patients with the intention of inducing antigen-specific T-and B-cell responses.

For such use in the generation of anti-tumor immune responses, the maturated DC's used should not only be capable of presenting the tumor antigen(s), but in addition should preferably also be capable of inducing of Th1-type CD4+ T cells and CD8+ cytotoxic T lymphocytes, of expressing costimulatory molecules, and have a migratory phenotype to migrate from the injection site to T-cell areas in lymph nodes where they can present the antigen(s) to T cells.

In the vaccines used to date, inflammatory mediators such as TNF-, IL-1, IL-6 and Prostaglandin E2 have been used to mature monocyte-derived DCs. However, activation of DC by solely pro-inflammatory cytokines yields DC's that support CD4+ T cell clonal expansion, but fail to efficiently direct helper T cell differentiation. DC polarize immune responses via secretion of soluble factors, such as cytokines (Kapsenberg, Nature Reviews Immunology 2003; 3:984-93). IL-12p70 favours the differentiation of IFNy producing T helper 1 cells, and is thus relevant in enhancing in vivo anti-tumor responses (Trinchieri, Nature Reviews Immunology 2003; 3:133-46; Kim et al., Cancer Immunology Immunotherapy 2006; 55:1309-1).

DC matured with only with proinflammatory cytokines do not produce IL-12p70

(Boullart et al., Cancer Immunology Immunotherapy 2008;DOI 10.1007/s00262-008-0489-2). In contrast, exposure of these cells to pathogen components generated DC that did produce IL-12p70 and promote T cell help (Mailliard, Cancer Research 2004; 64:5934-7; Sporri and Sousa, Nature Immunology 2005; 6:163-70).

Thus, the maturation stimuli and methods that have been used up to now to provide activated and antigen-loaded DC's for use in DC-based vaccines may not be completely satisfactory, and for example may not result in optimal Th1 responses or other functional characteristics that are desired for use in immunotherapy of tumors. This may be confirmed by the observation of the present inventors that when DC's that are activated with these known methods are used in clinical trials, sometimes only a limited number of clinical responses are observed, with some patients responding to DC vaccinations while others do not (see for example the following non-prepublished reference: Lesterhuis et al., Critical Reviews in Oncology Hematology, 2008, 66:118-134. It should however be noted that this observation is still the subject of further research, and that consequently, the present inventors do not wish to be limited to any specific explanation or hypothesis).

pDCs, as natural circulating DCs and main source of type I interferons, have also been proposed for use in DC-based vaccines. In mice, vaccination with pDCs confer protection against Leishmania major (Remer, Eur J Immunol. 2007 September; 37(9):2463-73). Furthermore pDCs in combination with myeloid DCs (mDCs), synergistically enhance the anti-tumor immune response. Revealing the capacity of pDCs to generate Ag-specific T cell responses themselves and also enhances the ability of mDCs to stimulate T cells.

More generally, as will be clear to the skilled person based on the disclosure herein, the antigen-presenting cells mentioned herein can give rise to, initiate, mediate or enhance various types of immune responses against the antigen(s) that they are presenting (e.g. with which they are “loaded”, as described herein).

As will be clear to the skilled person, all this makes loaded antigen-presenting cells (such as loaded dendritic cells) a promising means for therapy, and in particular for immunotherapy, of a range of different diseases and disorders, depending upon the antigen(s) that are present on the loaded antigen-presenting cells.

Thus, it is a general objective of the invention to provide antigen-presenting cells that are loaded with one or more desired antigens that can be used in the prevention and treatment of diseases and disorders in a subject and/or that can be used to generate an immune response in a subject against the one or more antigens present on the loaded antigen-presenting cells.

As already mentioned herein, the “antigen-presenting cells” or “APC's” may be any suitable antigen-presenting cell(s), and suitable antigen-presenting cells will be clear to the skilled person based on the disclosure herein. Generally, this may be any cell that presents and/or displays (or is capable of presenting and/or displaying) an antigen (such as a foreign antigen), e.g. so as to present it to (other) cells of the immune system such as T-cells or B-cells. Usually, an APC will present and/or display such an antigen on its surface (or is capable of doing so), often as a complex with a suitable receptor expressed by the APC, such as (in particular) the Major Histocompatability Complex (MHC, such as MHC class-I or MHC class-II).

The APC's that may be activated using the methods described herein may in particular be cells that can prime T-cells and/or that express MHC-class-II (sometimes also referred to as “professional APC's”); although the invention in its broadest sense is not limited thereto, and for example also includes APC's (such as DC's) that are (also) capable of triggering a B-cell mediated immune response. It should also be noted that generally, the invention is not particularly limited to any mechanism, explanation or hypothesis as to the manner in which, using the methods of described herein, the desired or intended immune response is generated. Thus, this may for example be a T-cell mediated immune response, a B-cell mediated immune response, or any other suitable immunological mechanism for generating an immune response; or any combination of the foregoing.

Some non-limiting APC's that may be activated and/or loaded using the methods described herein are dendritic cells, macrophages. B-cells and monocytes; as well as specialized cells in specific tissues or organs such as astrocytes/microglial cells (in the brain), Ito cells/Kupfer cells (in the liver), liver sinusoidal endothelial cells (LSEC), alveolar macrophages (in the lungs), osteoclasts (in bone), sinusoidal lining cells (in the spleen).

Thus, generally, the methods described herein can be used to activate and/or load one or more of these APC's, either systemically or at a specific site (such as in a specific tissue, organ or part) of the body of the subject to be treated. For example, when the methods described herein are used to treat a tumor, the methods described herein be used to activate and/or load one or more of these APC's either systemically or in the organ(s) or tissue(s) in which the tumour is present (e.g. by administration to said tissue or organ, and/or by administration into the tumor or into the immediate surroundings of the tumor).

The methods described herein may be used to activate and/or load one or more specific APC's in the tissue or organ in which they occur. For example, methods described herein may be used to activate and/or load astrocytes/microglial cells in the brain, Ito cells/Kupfer cells and/or liver sinusoidal endothelial cells (LSEC) in the liver, alveolar macrophages in the lungs, osteoclasts in bone, or sinusoidal lining cells in the spleen.

Other suitable APC's that can be activated and/or loaded using the methods described herein will be clear to the skilled person based on the disclosure herein.

Generally, when the methods described herein are used to activate (and thereafter optionally load) APC's in vitro or ex vivo, the methods described herein can be used to provide “clinical grade” activated (and optionally loaded) APC's, by which is meant activated (and optionally loaded) APC's that are suitable for administration to a human subject.

In one specific, but non-limiting aspect, the APC's will be dendritic cells, such as pDC's, mDC's or suitable precursors or progenitors thereof, as further described herein; including, without limitation DC's that have been cultured in vitro such as monocyte-derived DC's or CD34-derived DC, or DC that have been directly isolated from body fluids or tissues, as further described herein.

Accordingly, in the present specification herein, the invention will be described with particular reference to dendritic cells. However, based on the disclosure herein, it will be clear to the skilled person that the invention in its broadest sense is not limited thereto and may also be applied to other APC's (as described herein).

Generally, in order provide loaded DC's, the DC's must be both activated (as defined herein) as well as loaded with the one or more desired antigens (in practice, usually, the DC's are first activated and then loaded with the antigen). However, the means that are currently available for activating DC's have a number of drawbacks, in particular when the activated and loaded DC's are to be used for administration to a subject. Thus, it is a further objective of the invention to provide methods and means for activating DC's that do not have the drawbacks of the currently available means.

It is known in the art (from example from Gibson et al., supra) that DC's can be activated using either ligands (and in particular agonists) of the “toll-like receptors” or “TLR's” that are present on the DC's, or using small chemical compounds that act as agonists of TLR's (also known as “immune response modifiers” or “IRM's”). For a general description of TLR's and TLR signalling pathways (in particular on or in DC's), reference is again made to the prior art cited herein, as well as to for example the review by Kanzler et al., Nature Medicine, May 2007, Volume 13, No 5, 552-559 and by Baccala et al., May 2007, Volume 13, No 5, 543-551, as well as to for example Takeda and Akira, Seminars in Immunology, 16 (2004), 3-9; Akira and Takeda, Nat. Rev. Immunol., 4, 499-511 (2004), Akira et al., Cell, 124, 783-801 (2006); and Marschak-Rothstein, Nat. Rev. Immunol., 6, 823-835 (2006). However, such ligands and IRM's are either not readily available and/or may have safety concerns associated with their use for activating DC's that are intended for subsequent administration to a subject.

For example, pDCs have surface expression of Toll-like receptor 1 (TLR1), and endosomal expression of TLR7 and TLR9, and the stimulatory effects of bacterial and viral DNA are ascribed to the presence of unmethylated CpG oligonucleotide (ODN) motifs, which are recognized by TLR-7 and (predominantly) TLR-9. Synthetic oligonucleotides with unmethylated CpG motifs have been developend and used to mimic the immune-stimulatory effects of bacterial DNA on pDC's, and it has been described that such synthetic TLR agonists are very potent inducers of pDC activation.

It has also been described that activation using these synthetic TLR-ligands leads to anti-tumor responses and several phase I clinical trials have been initiated (Molenkamp et al., Clin Cancer Res. 2007 May 15; 13(10):2961-9).

The synthetic CpG ODNs that have been described in the art can be classified on the basis of their immunological effects on purified B cells and pDCs. Thus far, three classes of chemically modified CpG-ODNs with different sequence motifs have been developed: the A-, B-, and C- classes, which differ in their immune-stimulatory activity. CpG-A skews to the innate immune response by inducing production of type I IFNs by pDC, whereas CpG-B, a potent B cell stimulator and inducer of pDCs maturation, leads to adaptive immunity. The combination of structural elements of both CpG-A and CpG-B led to the synthesis of CpG-C which induces high amount of type I IFNs in pDCs and are also capable to activate and mature B cells and pDCs, respectively.

However, as mentioned above, these synthetic TLR agonists are not readily available nor proven safe or efficacious for use in providing DC-based vaccines that are intended for administration to patients.

Thus, there is a need in the art to provide safe and readily available means that can be used to provide activated DC's that can subsequently be loaded with one or more desired antigens and that thereafter can be used for prophylaxis or therapy in a subject.

According to one specifically preferred (but non-limiting) aspect of the invention, it has been found that commercially available vaccines (or suitable components or constituents thereof, as described herein) can conveniently be used to activate antigen-presenting cells (and in particular, but without limitation, dendritic cells). The vaccines used in the invention can in particular (but without limitation) be vaccines that are based on and/or derived from bacteria or viruses, such as inactivated or attenuated bacteria or viruses. Also, with advantage, the vaccines used in the invention are vaccines that are commercially available and/or approved for administration and use in human subjects, and thus are generally considered safe. Moreover, they are conveniently available in a ready-to-use form.In addition, according to a specific aspect of the invention, it has been found that by specific selection of the vaccine used for such activation, it is not only possible to activate the antigen-presenting cells, but also to regulate the nature of the response of the activated pDC's, i.e. towards (increased) production of cytokines (such as, in particular, Type I IFN's such as IFN-alpha); towards the ability to differentiate (i.e. activate and/or mature) B-cells and in particular pDC's (for example, determined by measuring the upregulation of co-stimulatory molecules such as, in particular, CD80 and/or CD86, see for example Examples 3B and 4A); or towards both (increased) production of cytokines as well as the ability to differentiate B-cells and/or pDC's (with the latter “dual action” usually being preferred, although the invention is not limited thereto).

In the work that has led to the present invention, the inventors have tested a number of commercially available vaccines for their capacity to induce pDC activation (see Table 1 below). As can be seen from the Experimental Section below, some of the vaccines tested INFANRIX, BMR, Rabies) were found to have the ability to induce IFN-a production; whereas Act-Hib and BCG were found to have an ability to induce the differentiation of pre-pDCs into pDCs (as measured by the ability to induce the antigen-presenting molecules CD80 and CD86) but were not able to induce highly increased levels of type I IFNs. FSME was found to be able to induce both IFN-a production and phenotypic maturation of pDCs.

Thus, the invention not only provides means for activating pDC's, but also means for directing the response of the pDC's towards a response that is similar to the response of pDC's to the synthetic ODN GpC-A (i.e. towards IFN-alpha production), towards a response that is similar to the response of pDC's to the synthetic ODN GpC-B (i.e. towards maturation and upregulation of antigen presenting molecules such as CD80 and CD86)); or towards a response that is similar to the response of pDC's to the synthetic ODN GpC-C (i.e. towards both Type I IFN production as well as phenotypic maturation of pDC's and induction of co-stimulatory molecules such as CD80 and CD 86).

The inventors have also found that when pDC's are simultaneously incubated with the vaccines used in the present invention, but in the additional presence of chloroquine (a compound which is known to prevent endosomal maturation, primarily through inhibition of vesicular acidification (see for example Lande, Nature. 2007 Oct. 4; 449(7162):564-9), both the above-described secretion of IFN-a secretion as well as the above described differentiation of pDCs which were found to occur without the presence of chloroquine were both found to be inhibited or reduced. One possible explanation for this is that the advantageous effect of the vaccines used in the invention on the activation of pDC's is mediated by endosomal maturation and potentially involves binding of antigenic components in the vaccines to endosomal TLR's, such as TLR-7 and in particular TLR-9 (although the inventors do not wish to be limited to any specific mechanism, hypothesis or explanation).

It was further found that when pDC's are simultaneously incubated with the vaccines used in the present invention, but that in the additional presence of the synthetic ODN TTAGGG (an antagonist of TLR-9), the ability of the vaccines used to activate pDC's (either by increasing IFN-alpha production, phenotypic maturation of pDC's, or both, depending on the vaccine used), was inhibited. This further strengthens the hypothesis that the advantageous effect of the vaccines used in the invention on the activation of pDC's is mediated endosomal TLR's, such as TLR-7 and in particular TLR-9 (although it should again be noted that the inventors do not wish to be limited to any specific mechanism, hypothesis or explanation).

It was also found that the ability of the vaccines tested to induce pDC's was independent of the adjuvants present in the vaccine (data not shown).

Thus, in a preferred, but non-limiting, specific aspect, the invention relates to a method for providing an activated (as defined herein) antigen-presenting cell (and in particular, but without limitation, dendritic cell), and/or a composition that comprises at least one activated (as defined herein) antigen-presenting cell (and in particular, but without limitation, dendritic cell), which method at least comprises the steps of:
a) providing a composition that comprises at least one antigen-presenting cell (i.e. one or more antigen-presenting cell s, and in particular a population of antigen-presenting cells, such as a population of antigen-presenting cell s with a size that is sufficient for the purposes of immunotherapy);
b) contacting said composition with a vaccine (i.e. in such a way that the antigen-presenting cell is activated as defined herein).

As mentioned above, in this method, the antigen-presenting cell may be any desired or intended antigen-presenting cell, but may in particular be a dendritic cell (as further described herein).

The invention also relates to a composition that comprises at least one antigen-presenting cell (and in particular, but without limitation, dendritic cell) that has been activated (as defined herein) using a vaccine and/or using one of the methods described herein. As will be clear from the further description herein, such a composition (and/or the APC's present therein) are preferably such that it is suitable for administration to a subject, for example in methods for immunotherapy as described herein.

The invention further relates to a antigen-presenting cell (and in particular, but without limitation, dendritic cell) that has been activated using a vaccine and/or using one of the methods described herein, and to compositions comprising at least one such activated antigen-presenting cell.

The invention further relates to the use of a vaccine in the preparation of a composition that comprises at least one activated antigen-presenting cell (and in particular, but without limitation, dendritic cell), and also to the use of a vaccine in activating a antigen-presenting cell (and in particular, but without limitation, a dendritic cell).

The invention also relates to a vaccine for (use in) activating dendritic cells and/or in preparing a composition that comprises at least one dendritic cell. The invention also relates to a method for activating (as defined herein) an antigen-presenting cell (and in particular, but without limitation, dendritic cell), which method comprises contacting the antigen-presenting cell with one or more antigenic components (as defined herein) that are derived from a vaccine, wherein the contacting of the antigen-presenting cell with the antigenic component(s) is performed by contacting a composition that comprises the antigen-presenting cell with a vaccine that comprises the antigenic component(s). As mentioned herein, the antigenic component(s) may for example be an attenuated, weakened or inactivated bacterium, virus or virus particle (i.e. as present in the vaccine and/or suitable for use in a vaccine) or a nucleic acid present in or encoded by such a virus or bacterium. Examples of such vaccines, antigenic components, bacteria and viruses will become clear from the further description herein.

The invention further relates to applications and uses of an antigen-presenting cell (and in particular, but without limitation, dendritic cell) that has been activated using one of the methods described herein, and to applications and uses of compositions comprising such an activated antigen-presenting cell. Such applications and uses will become clear to the skilled person based on the further disclosure herein. In particular, as mentioned above, the activated antigen-presenting cells obtained using the methods described herein can be loaded with one or more desired antigens in order to provide activated and loaded antigen-presenting cells (and in particular, but without limitation, activated and loaded dendritic cells) that can for example be used in methods for immunotherapy, as further described herein.

By “loading the APC (or DC) with antigen(s)” is generally meant any process whereby an antigen-presenting cell (i.e. an APC or DC that has been suitably activated as defined herein) is treated with one or more antigens (or with nucleic acids that encode the one or more antigens) so as to make the APC capable of presenting the antigen(s) to T-cells, and/or to B-cells, and/or more generally of raising a specific immune response against said antigen(s) (optionally after the cell has suitably processed said antigen). This is usually performed by contacting or treating the APC's with the one or more antigens (or with one or more nucleic acids that encode the one or more antigens) in such a way that the (activated) APC's will carry or express the antigen(s), i.e. on their surface.

For example, for this purpose, the activated antigen-presenting cells (such as dendritic cells) may be pulsed or otherwise contacted with the one or more antigens in such a way that the antigens bind to the surface of the APC's (and/or to a receptor, complex or protein present on the surface of the activated APC's, such as the MHC complex, and in particular but without limitation, when the APC is a professional APC, the MHC Class-II complex). This can be performed in any suitable manner and using any suitable technique known per se to the skilled person.

Alternatively, it is also possible to transform or transfect (e.g. transiently) the APC's (such as DC's) with a nucleic acid that encodes the antigen(s), such that the antigen(s) are expressed on the surface of the APC's. This may for example be performed by using electroporation, suitable viral vectors (such as viral vectors for gene therapy known per se), methods and techniques known per se, which will be clear to the skilled person. However, it will be clear to the skilled person that the use of viral vectors will usually be more cumbersome than simply contacting the activated APC's with the antigen(s) of interest, so that the latter will generally be preferred. Also, when the activated and loaded APC's are to be returned to a subject (e.g. in methods for immunotherapy), the use of APC's that have been treated with viral vectors may (again) cause safety concerns.

It should also be noted that when a viral vector is used to load the APC with the desired antigen(s) (i.e. by transforming or transfecting the APC's with a nucleic acid encoding the antigen), the viral vector used may further be such that it also activates the APC (i.e. serves as an antigenic component, as further described herein). According to this aspect of the invention, the method for activating and loading the APC's may thus at least comprise a (single) step of contacting the APC's (such as DC's) with a virus, viral particle, viral vector (such as a viral nucleic acid) or any other virus-derived composition or preparation (such as a viral lysate, fragment, fraction, supernatant or suspension) that is capable of activating the APC's (as described herein) and that encodes the desired antigen(s) (and/or contains or comprises a nucleic acid that encodes the desired antigen(s)), such that the APC's are activated (as further described herein) and such that the APC's are transformed or transfected with a nucleic acid that encodes the desired antigen(s), in particular such that the APC's are loaded (as described herein) with the desired antigen(s). Accordingly, this aspect of the invention further relates to a virus, viral particle, viral vector (such as a nucleic acid, for example a gene therapy vector) or other virus-derived composition or preparation that is capable of activating an APC (and in particular, a DC) and that is capable of loading the APC's (and in particular, DC's) with one or more desired antigens (i.e. that encodes the desired antigen(s) and/or contains or comprises a nucleic acid that encodes the desired antigen(s) and that is capable of transforming or transfecting the APC's (and in particular, DC's) with a nucleic acid encoding the desired antigen(s), such that the APC's express the desired antigen(s)).

For a further description of “antigen loading” of APC's such as dendritic cells, and of peptide/protein based techniques and genetic techniques that can be used to load APC's (and in particular, DC's) with a desired antigen, reference is for example also made to the review by Tuyaerts et al. cited herein.

Thus, in another aspect, the invention relates to the use of an antigen-presenting cell (and in particular, but without limitation, dendritic cell) that has been activated by one of the methods described herein, in preparing an antigen-presenting cell (and in particular, but without limitation, dendritic cell) that has been loaded with one or more antigens, and/or in preparing a composition that contains such an activated and loaded an antigen-presenting cell.

In another aspect, the invention relates to a method for providing an activated antigen-presenting cell (and in particular, but without limitation, dendritic cell) that has been loaded with one or more desired antigens, and/or a composition that comprises an activated antigen-presenting cell (and in particular, but without limitation, dendritic cell)that has been loaded with one or more desired antigens, which method comprises at least the steps of:
a) providing a composition that comprises at least one antigen-presenting cell;
b) contacting said composition with a vaccine so as to activate (as defined herein) said at least one antigen-presenting cell; and
c) loading (as defined herein) the activated antigen-presenting cell with the one or more desired antigens.

Again, in this method, the antigen-presenting cell may be any desired or intended antigen-presenting cell, but may in particular be a dendritic cell (as further described herein).

In the above method, after step b) and before step c), (the composition comprising) the APC's may be treated or washed in order to remove the antigenic component (or any excess thereof) and/or excess of the activating composition.

The invention also relates to a composition that comprises at least one antigen-presenting cell (and in particular, but without limitation, dendritic cell) that has been activated (as defined herein) using one of the methods described herein and loaded (as defined herein) with one or more desired antigens (e.g. also using the methods described herein). Again, such a composition (and/or the APC's/DC's present therein) are preferably such that it is suitable for administration to a subject, for example in methods for immunotherapy, as described herein. It will be clear to the skilled person that for this purpose, the antigen(s) loaded onto the APC's should most preferably also be suitable for administration to a subject, and more preferably be suitable for use in methods for immunotherapy, as described herein.

The invention further relates to an antigen-presenting cell (and in particular, but without limitation, dendritic cell) that has been activated and loaded with one or more desired antigens using one of the methods described herein, and to compositions comprising at least one such activated and loaded antigen-presenting cell.

The invention further relates to the use of a vaccine in the preparation of a composition that comprises at least one activated and loaded antigen-presenting cell (and in particular, but without limitation, dendritic cell), and also to the use of a vaccine in preparing an activated and loaded antigen-presenting cell.

The invention also relates to a vaccine for (use in) preparing activated and loaded antigen-presenting cell (and in particular, but without limitation, dendritic cell).

The invention also relates to a method for activating (as defined herein) and loading (as defined herein) an antigen-presenting cell (and in particular, but without limitation, a dendritic cell), which method comprises (i) activating the antigen-presenting cell by contacting the antigen-presenting cell with one or more antigenic components (as defined herein) that are derived from a vaccine, wherein the contacting of the antigen-presenting cell with the antigenic component(s) is performed by contacting a composition that comprises the antigen-presenting cell with a vaccine that comprises the antigenic component(s); and (ii) loading antigen-presenting cell with one or more antigens (preferably after the dendritic cell has been activated and the one or more antigenic components have been removed, i.e. by washing). The invention further relates to applications and uses of an antigen-presenting cell (and in particular, but without limitation, dendritic cell) that has been activated and loaded using one of the methods described herein, and to applications and uses of compositions comprising such activated and loaded antigen-presenting cells. Such applications and uses will become clear to the skilled person based on the further disclosure herein, and will mainly depend on the antigen(s) with which the antigen-presenting cell has been loaded. For example, as further described herein, the loaded antigen-presenting cells may be used to generate a cytotoxic or other immune response against the antigen(s) and/or in methods for immunotherapy in which such a cytotoxic response (or other desired immune response) against the antigen(s) is to be raised. One specific, but non-limiting use is in methods for immunotherapy of tumours/cancer, by using antigen-presenting cell (and in particular, but without limitation, dendritic cells) that have been activated and loaded (i.e. using the methods described herein) with an antigen that is specific for the tumor against which an immune response is to be raised (i.e. an antigen that is expressed on the surface of the tumor cells).

When the antigen-presenting cells used in the methods described herein are dendritic cells, they can be any suitable or desired dendritic cell, such as body fluid or tissue derived pDC's, mDC's or DC's cultured from suitable precursors or progenitors such as monocytes or CD34+ cells (such as mDC's cultured from monocytes or CD34+ cells). In this respect, it is remarked that although in current methods for immunotherapy, usually mDC's are used, the methods described herein can equally efficaciously be used with pDC's, so that the methods described herein further contribute to establishing the use of pDC's as a viable alternative to the use of mDC's.

When the antigen-presenting cells used in the methods described herein are dendritic cells (either pDC's, mDC's, or monocyte-derived DC's), they may be obtained from any suitable source, such as from any mammal and in particular from a human subject, using any suitable technique known per se. The DC's may also be obtained by in vitro cultivation, for example starting from a sample of DC's or progenitors or precursors for DC's that has been obtained from a mammal or human subject. For example, and without limitation, when it is intended to administer the DC's to a subject (for example for methods for immunotherapy as described herein), the DC's may be DC's that have been obtained from said subject and/or that have been obtained by in vitro cultivation starting from a sample of DC's obtained from said subject. Suitable methods and techniques for obtaining and cultivating DC's are well known to the skilled person. Reference is for example made to Adoptive Immunotherapy: Methods and Protocols, (edited by B. Ludewig and M. W. Hoffman), from the series “Methods in Molecular Medicine”, Humana Press (2004). Reference is also made to the review by Tuyaerts et al., “Current approaches in dendritic cell generation and future implications for cancer immunotherapy”, Cancer Immunol Immunother. 2007 May 15; e-publication ahead of print, PMID: 17503040.

As the preferred methods described herein are meant to activate (as defined herein) DC's, the DC's that are used as a starting material in the methods described herein are preferably in a non-activated state, and may for example be immature and/or undifferentiated DC's (and in particular immature and/or undifferentiated pDC's). However, it should be noted that the invention in its broadest sense is not limited thereto and generally encompasses any suitable and/or appropriate use of the methods described herein to provide activated DC's and/or to provide DC's that can be loaded with one or more antigens. The same applies to the activation of other APC's using the methods described herein, where said APC's also occur in a non-activated state (such as an immature and/or undifferentiated state).

By “activating” DC's (or more generally APC's, where applicable) is generally meant herein the steps or the process of bringing DC's (or APC's) into a state in which they have the capacity of initiating an immune response, and in particular of stimulating T-cells and/or a T-cell mediated response (and/or stimulating B-cells and/or a B-cell mediated response). More in particular, “activating” DC's (or more generally APC's, where applicable) can involve bringing DC's (or APC's) into a state in which they can be loaded (as described herein) with one or more desired antigens and subsequently used to present these antigens to T-cells (and in particular killer T-cells) or B-cells, most preferably in such a way that they can initiate and/or stimulate a T-cell (and/or B-cell) mediated response against said antigen(s).

As will be clear to the skilled person based on the disclosure and prior art cited herein, when the DC's (or more generally APC's, where applicable) that are used as the starting material are immature or undifferentiated DC's (such as immature or undifferentiated pDC's and mDC's, for example the pDC's and mDC's that are present in the blood, which can be considered as “precursor” DC's, see Gibson et al., supra), “activating” of the DC's will usually involve (further) maturation and/or differentiation of the DC's. Also, when the DC's that are used as the starting material are not (sufficiently) capable of upregulating CD80 and/or CD86, “activating” of the DC's will usually mean that the DC's are brought into a state in which they can (sufficiently) upregulate CD80 and/or CD86. Similarly, when the DC's that are used as the starting material are not capable of producing cytokines (or do not produce cytokines at a level that is sufficient to stimulate T-cells), “activating” of the DC's will usually mean that the DC's are brought into a state in which they produce such cytokines (i.e. at a level that is sufficient to stimulate and skew T-cells). For example, and without limitation, activation of mDC's (or progenitors or precursors for mDC's) may involve bringing the mDC's into a state where they produce (amongst other cytokines) IL-12, whereas activation of pDC's (or precursors for pDC's) may involve bringing the pDC's into a state where they produce (amongst other cytokines) interferons such as Type I IFN's (IFN-alpha/beta). More generally, “activation” of the DC's may involve increasing the ability of the DC's to stimulate and skew T-cells, whether via (increased) production and secretion of cytokines, via (increased) upregulation of CD 80 and/or CD86, and/or via any other suitable biological mechanism or action.

The vaccines used in the methods described herein can be any suitable vaccine that is capable of activating (as defined herein) the intended or desired antigen-presenting cell(s) (and in particular, but without limitation, dendritic cells). Preferably, said vaccines comprise one or more antigens or antigenic components that are capable of activating (as defined herein) the intended or desired antigen-presenting cell(s), which antigens or antigenic components may in particular be as further defined herein.

In particular, the vaccines used herein may be formulations or preparations of such antigens or antigenic components that comprise the one or more antigens or antigenic components and at least one pharmaceutically acceptable carrier, such as water, a physiological (usually aqueous) solution or buffer, or another (aqueous) medium that is suitable for administration to a human subject. The vaccines used herein may in particular be in the form of injectable solutions or suspensions or in the form of a lyophilized preparation that can be reconstituted into an injectable preparation or suspension immediately prior to use. It will also be clear that from a practical standpoint, vaccines that are in the form of injectable preparations or suspensions (or that can be reconstituted into an injectable preparation or suspension) are also convenient for use in the present methods, as they can easily be added to and mixed with a suspension of the dendritic cells.

When the vaccines used herein are in the form of a formulation or preparation, they may be in a ready-to-use form (or in a form that can be constituted into a ready-to-use form). Also, the vaccines used herein may be contained in a suitable container (such as a flask, vial, bag or syringe) that may be packaged together with instructions for use of the vaccine in therapy or prophylaxis in human subjects or with a product information leaflet.

The vaccines used herein are preferably safe for use in or in connection with human subjects, and may in particular be formulations or preparations that are approved for use in or in connection with human subjects. As such, the vaccines used herein may for example be commercially available formulations or preparations.

For example, and without limitation, one of the following commercially available vaccines may be used: FSME-Immun™ (a vaccine containing inactivated FSME, a tick-borne encephalitis virus) made by Baxter AG; PNEUMO-23™ (a vaccine against Streptococcus pneumoniae (pneumococcus) prepared from purified pneumococcal capsular polysaccharide antigens) made by Aventis Pasteur MSD; INFANRIX-IPV (a vaccine against diphtheria, tetanus and Bordetella pertussis, based on diphteria and tetanus toxoids and the acellular Pertussis antigens PT, FHA and pertactin) GlaxoSmithKline; INFLUVAC™, (an Influenza vaccine based on influenza surface antigens (haemagglutinin and neuraminidase)) made by Solvay Pharma; TYPHIM (a vaccine against typhoid fever containing the Vi polysaccharide antigen of Salmonella typhi) made by Sanofi Pasteur MSD; the Tetanus vaccine made by the Netherlands Vaccine Institute (NVI), the Netherlands (which contains Tetanus immunoglobulin); ACT-HIB™ (a vaccine against influenza containing a Haemophilus b conjugate with tetanus toxoid) made by Sanofi Pasteur MSD; HBVAXPRO™ (a hepatitis B vaccine containing a hepatitis B virus surface antigen) made by Sanofi Pasteur MSD; BCG (a vaccine against tuberculosis containing an attenuated strain of Mycobacterium bovis) made by the NVI, The Netherlands; NEISVAC-C (a meningitis C vaccine containing a Neisseria meningitidis 1 Group C polysaccharide conjugate); HIB (a meningitis vaccine containing a capsular polysaccharide extracted from culture of Haemophilus influenza type b) made by GlaxoSmithKline; PREVENAR (a vaccine against Streptococcus pneumoniae (pneumococcus) containing a pneumococcal polysaccharide conjugate) made by Wyeth; the “BMR vaccine” (mumps, measles and Rubella vaccine containing attenuated mumps, measles and Rubella virus) made by the Netherlands Vaccine Institute (NVI), the Netherlands; HAVRIX™ (a hepatitis A vaccine containing inactivated Hepatitis A vaccine) made by GlaxoSmithKline; STAMARIL (a vaccine against yellow fever containing an attenuated form of the yellow fever virus) made by Sanofi Pasteur MSD, and the yellow fever vaccine YF-17D (see Querec et al., JEM, Vol. 203, No. 2, 413-424 (2006). Other suitable vaccines will be clear to the skilled person based on the disclosure herein, and for example include (without limitation) the vaccines mentioned in Tables 2-5 of the review by Kanzler et al., supra.

Table 1 gives a list of some of the vaccines that can be used in the practice of the invention

TABLE 1
Vaccines based on bacteria

| Infectious agent | Vaccine | Disease | Type of vaccine | Supplier | Adjuvant |
|---|---|---|---|---|---|
| bacteria | | | | | |
| Salmonella typhi | TYPHIM Vi | Typhoid fever | polysaccharide | Sanofi pasteur | none |
| Haemophilis influenzae | ACT-HIB | Meningitis, epiglottitis, pneumonia | conjugated | Aventis | Tetanus toxoid |
| type b | | type b | | Pasteur | |
| Mycobacterium bovis | BCG | Tuberculosis | live attenuated | NVI | none |
| bacillus | | | | | |
| viruses | | | | | |
| Encephalitis virus | FSME | Lyme disease | inactivated | Baxter | AlOH |
| Rabies virus | Rabies | Rabies | inactivated | Sanofi Pasteur | Neomycin |
| Measles virus | BMR | German measles, Respiratory tract | Live attenuated | NVI | none |
| Mumps virus | | infection, mumps, meningitis, | | | |
| Rubella virus | | orchitis | | | |
| Difteria | INFANRIX- | Difteria | subunit, inactivated, | Glaxo | AlPO4, AlOH |
| | IPV | Tetanus toxoid | conjugated | SmithKline | |
| Clostridium tetani | +HIB | Pertussis | | | |
| Acellulair pertussis | | Poliomyelitis, paralysis | | | |
| Poliovirus | | Meningitis, epiglottitis, pneumonia | | | |
| | | type b | | | |
| Haemophilis influenzae | | | | | |
| type b | | | | | |
| Influenzavirus A | INFLUVAC | Flu, respiratory diseases | inactivated | Solvay Pharma | none |
| Influenzavirus B | 2006-2007 | | | | |

Generally, the activation/maturation of the DC's that is achieved by applying the methods described herein can be determined in any manner known per se, which will usually comprise measuring one or more properties or parameters (or suitable combination thereof) of the DC's that are known to be associated with mature DC's (i.e. that are induced and/or that change as DC's mature). Examples of such properties and parameters, and methods and assays for measuring these properties, will be clear to the skilled person, for example based on the disclosure and examples herein). These for example include, without limitation:

A) in case of pDC's:
(increased) ability of the pDC's to activate T-cells. In particular, the pDC's obtained by the invention should not only be capable of inducing a Th2 response (i.e. inducing Th2 cell development, (see for example Liu, in Cell , 106:259-262, 2001), but preferably a Th1 response as well. This may for example be determined by measuring the ability of the pDC's to induce the production of cytokines (such as IFN-gamma, TNF-alpha and/or IL-2 by T-cells (see for example Example 3C below);
(increased) expression by the pDC's of costimulatory molecules such as CD80, CD86, CD83, MHC class-I and/or MHC-class II (see for example Example 3B below);
(increased) production by the pDC's of cytokines such as (in particular) IFN-alpha (see for example Example 3C);
(increased) random migration and/or CCR-7 mediated migration (see for example Example 3D below) and/or an increased expression by the pDC's of receptors involved in chemotaxis (such as CCR-7);
(increased) capacity of the pDC's to stimulate allogeneic T-cells (see for example Example 3E below);
(increased) ability of the pDC's to present antigens to effector lymphocytes (such as CD4+ cells, CD8+ cells and also to B-cells), for example as determined by measuring specific responses of such cells to pDC's that have been loaded with a suitable antigen (for example, a model antigen such as keyhole limpet hemocyanin (KLH), see for example Example 3F) and/or an (increased) ability to induce proliferation of autologous T-cells (see again for example Example 3F below);

or any suitable combination thereof.

B) in case of mDC's:
(increased) ability of the mDC's to activate T-cells. In particular, the mDC's obtained by the invention should not only be capable of inducing a Th2 response, but preferably a Th1 response as well. This may for example be determined by measuring the ability of the mDC's to induce the production of cytokines (such as IFN-gamma, TNF-alpha and/or IL-2) by T-cells (see for example Example 41 below);
(increased) expression by the mDC's of costimulatory molecules such as CD80, CD86, CD83, MHC class-I and/or MHC-class II (see for example Example 4A below);
(increased) production by the mDC's of cytokines such as (in particular) IL-12p70 (see for example Examples 4H and 4J below);
(increased) migration/chemotaxis by the mDC's, such as random migration on fibronectin or CCR-7 mediated migration (see for example Example 4F below) and/or an increased expression by the pDC's of receptors involved in chemotaxis (such as CCR-7);
(increased) capacity of the mDC's to stimulate allogeneic T-cells (see for example Example 41 below);
(increased) ability of the mDC's to present antigens to effector lymphocytes (such as CD4+ cells, CD8+ cells and also to B-cells), for example as determined by measuring specific responses of such cells to mDC's that have been loaded with a suitable antigen (for example, a model antigen such as keyhole limpet hemocyanin (KLH), see for example Example 4J below) and/or an (increased) ability to induce proliferation of autologous T-cells (see again for example Example 4J below); or any suitable combination thereof.

Preferably, by using the methods described herein, said properties of the DC's are induced or increased/improved to levels that make the DC's obtained using the methods described herein suitable of their intended use, as further described herein. In this respect, it should for example again be noted that, as mentioned above, prior art methods and techniques for activating DC's do not always lead to the desired or intended combination of properties, in particular when the DC's obtained are to be used for immunotherapy of cancer.

In one preferred, but non-limiting aspect, the vaccine used in the methods described herein is such that, when the vaccine is contacted with the DC's to be activated, it is capable of increasing the production by the DC's of cytokines that are usually produced by such (activated) DC's (such as Type I interferons and in particular of IFN-alpha in the case of pDC's, and IL-12p70 in the case of mDC's), i.e. by at least 1%, preferably by at least 10%, such as by at least 20%, for example by 50% or more, compared to the DC's before they are contacted with the vaccine. This may for example be determined as described in the Experimental Section below. This aspect of the invention has been found to be particularly suited for the activation of pDC's, but can also be used for the activation of mDC's. Examples of such vaccines will be clear to the skilled person based on the disclosure herein.

In one specific aspect, the vaccine used is capable of increasing the production of Type I interferons without substantially inducing the maturation of the DC's.

In another preferred, but non-limiting aspect, the vaccine used in the methods described herein is such that, when the vaccine is contacted with the DC's to be activated, it is capable of inducing the maturation of pre-DC's into mature DC's (and in particular into pDC's), as measured by the upregulation (i.e. increased expression) of the costimulatory molecules CD80, CD83 and/or CD86 and increased expression of the antigen presenting molecules MHC class I and MHC class II by the DC's (i.e. by at least 1%, preferably by at least 5%, such as by at least 10%, for example by 25% or more, compared to the DC's before they are contacted with the vaccine). Again, this may for example be determined as described in the Experimental Section below. This aspect of the invention has been found to be particularly suited for the activation of pDC's, but can also be used for the activation of mDC's. Examples of such vaccines will be clear to the skilled person based on the disclosure herein. In one specific aspect, the vaccine used is capable of inducing the maturation of the DC's without substantially increasing the production of Type I interferons by the activated DC's.

In yet another preferred, but non-limiting aspect, the vaccine used in the methods described herein is such that, when the vaccine is contacted with the DC's to be activated, it is capable of both increasing the production by the DC's of cytokines that are usually produced by such (activated) DC's (such as Type I interferons and in particular of IFN-alpha in the case of pDC's, and IL-12p70 in the case of mDC's), as well as of inducing the maturation of pre-DC's into DC's (and in particular pDC's), as measured by the upregulation (i.e. increased expression) of the costimulatory molecules CD80 and/or CD86 and increased expression of the antigen presenting molecules MHC class I and MHC class II by the DC's (i.e. by at least 1%, preferably by at least 5%, such as by at least 10%, for example by 25% or more, compared to the DC's before they are contacted with the vaccine). Again, this may for example be determined as described in the Experimental Section below. This aspect of the invention has been found to be particularly suited for the activation of pDC's, but can also be used for the activation of mDC's. Examples of such vaccines will be clear to the skilled person based on the disclosure herein, and include FSME. Also, in the practice of the present invention, the use of vaccines that are capable of both increasing IFN Type I production as well as inducing pDC maturation will usually be preferred, although the invention is not limited thereto.

It should also be noted that it is possible in the invention to activate DC's by using two or more different vaccines, and that in doing so, a synergistic effect may be obtained. For example, when two or more different vaccines are used, at least one vaccine may be used that is capable of increasing the production of Type I interferons such as IFN-alpha, and at least one other vaccine may be used that is capable of inducing DC maturation. Other combinations of suitable vaccines (such as the vaccines described herein) may also be used. When DC's are activated according to the methods described herein using two or more different vaccines, the DC's to be activated may be contacted with a mixture of the two or more vaccines, but it is usually preferred to contact the DC's simultaneously with the two or more vaccines or to contact the DC's with the two or more different vaccines in two separate steps (usually performed shortly after one another).

It is also possible to use, in addition to the vaccine or combination of vaccines used, to use one or more vaccines as described herein in combination with one or more cytokines (such as TNF-alpha, IL-6 and/or IL-lbeta, and/or other pharmaceutically acceptable cytokines that have been used in the art to stimulate pDC's or mDC's, respectively) and/or one or more suitable hormones such as prostaglandins (for example Prostaglandin E2. These may be mixed with the vaccine(s) used, or the vaccines and the cytokines and/or hormones may be contacted with the DC's to be activated simultaneously or in separate steps (usually performed shortly after one another).

In the practice of the invention, it has been found that the use of a vaccine that is capable of both increasing the production of Type I interferons (and in particular, of IFN-alpha) by the pDC's (i.e. by at least 1%, preferably by at least 10%, such as by at least 20%, for example by 50% or more, compared to the DC's before they are contacted with the vaccine) as well as inducing the maturation of pre-DC's into pDC's, as measured by the upregulation (i.e. increased expression) of the costimulatory molecules CD80 and/or CD86 by the pDC's (i.e. by at least 1%, preferably by at least 5%, such as by at least 10%, for example by 25% or more, compared to the pDC's before they are contacted with the vaccine), and increased expression of the antigen presenting molecules MHC class I and MHC class II, such as the use of FSME, is particularly advantageous for the activation of pDC's. For the activation of mDC's, although single vaccines such as, without limitation, BCG, Act-HIB or Typhim can be used, the use of mixtures of vaccines or activation using two different vaccines (such as BCG and at least one other vaccine, for example BCG in combination with Typhim, Influvac and/or Act-HIB) has been found to be particularly advantageous, in particular in respect of the properties that are desired for activated mDC's that are to be used for the immunotherapy of tumors (see further herein).

Table 2 below shows the upregulation of CD80 and CD86 in pDC's (as determined by flow cytometry, mean fluorescence intensity is depicted) by some of the vaccines that can be used in the present invention.

TABLE 2
Upregulation of CD80 and CD86 by
vaccines used in the invention.

| Stimulus | CD80 | CD86 |
|---|---|---|
| IL-3 (−control) | 25 | 32 |
| CpG-C (+control) | 95 | 86 |
| TYPHIM Vi | 22 | 28 |
| BCG | 62 | 52 |
| ACT-HIB | 62 | 57 |
| FSME | 77 | 37 |
| Rabies | 33 | 21 |
| BMR | 35 | 34 |
| INFANRIX-IPV | 14 | 8 |
| INFLUVAC | 25 | 32 |

In addition, the vaccines used in the methods of the invention are preferably such that, and the methods described herein are preferably performed such that:
a) the resulting DC's are have the ability (or an improved ability) to migrate from the injection site to T cell areas in lymph nodes where they can then present the antigen to T cells, as may for example be determined by measuring the kinetics of acquisition of migratory function (for example using the chemotaxis assay or, in the case of mDC's, the random migration assay described in the Experimental Part below). This ability to migrate is preferably such that the resulting DC's are suitable for use in cancer immunotherapy. The migratory capacity of the DC's obtained using the methods of the invention may further be increased by adding a prostaglandine such as PGE₂; and/or
b) the resulting DC's are have the ability (or an improved ability) to produce the cytokines that are usually produced by such (activated) DC's (such as Type I interferons and in particular of IFN-alpha in the case of pDC's, and IL-12p70 in the case of mDC's), as may for example be determined using the cytokine detection assays like ELISA's or cytokine detection bead assays described in the Experimental Part below. This ability to produce Type I IFN is preferably such that the resulting DC's are suitable for use in cancer immunotherapy; and/or
c) the resulting DC's are have the ability (or an improved ability) to induce Th1-type CD4+ T cells and CD8+ cytotoxic T lymphocytes, as may for example be determined using T cell stimulation assays (i.e. primary inductions, mixed lymphocyte reaction, stimulation of antigen specific T cell lines) described in the Experimental Part below. This ability to induce of Th1-type CD4+ T cells and CD8+ cytotoxic T lymphocytes is preferably such that the resulting DC's are suitable for use in cancer immunotherapy; and/or
d) the resulting DC's are have the ability (or an improved ability) to express co-stimulatory molecules such as [CD80 and CD86] and have the ability (or an improved ability) to express of the antigen presenting molecules MHC class I and MHC class II, as may for example be determined using the flow cytometric assays described in the Experimental Part below. This ability to express co-stimulatory molecules is preferably such that the resulting DC's are suitable for use in cancer immunotherapy; and/or
e) the resulting DC's are have the ability (or an improved ability) to induce a Th1 response, as may for example be determined using the cytokine bead or cytokine ELISA assays described in the Experimental Part below. This ability to induce a Th1 response is preferably such that the resulting DC's are suitable for use in cancer immunotherapy.

Thus, activated DC's (i.e. either pDC's or mDC's, and activated using the methods described herein, i.e. using one or more vaccines and/or one or more antigenic components derived therefrom) that have the ability to migrate to T cell areas in lymph nodes, to produce cytokines that are usually produced by such (activated) DC's (such as Type I interferons and in particular of IFN-alpha in the case of pDC's, and IL-12p70 in the case of mDC's), to induce Th1-type CD4+ T cells and CD8+ cytotoxic T lymphocytes, that show expression (or increased expression) of co-stimulatory molecules such as CD80 and CD86, and expression (or increased expression) of the antigen presenting molecules MHC class I and MHC class II, and/or that have the ability to induce a Th1 response (for example, the ability to induce production of IFN-gamma by T-cells) in addition to the ability to induce a Th2 response (all of the foregoing preferably such that the DC's are suitable for use in cancer immunotherapy) form a further aspect of the invention. Such DC's are preferably loaded with antigen and capable of presenting said antigen; and for use in cancer immunotherapy may in particular be loaded with one or more tumor antigens or a mixture of tumor antigens, as further described herein.

In particular, using the methods of the invention, DC's are obtained (i.e. either pDC's or mDC's, and activated using either one or more vaccines and/or one or more antigenic components derived therefrom, as further described herein) that have one, preferably any combination or any two or more of, and preferably all of the following properties (in addition to an upregulation of costimulatory molecules such as CD80, CD86, CD83, MHC class-I and/or MHC-class II and an (increased) ability to present antigens to effector lymphocytes):
in a transwell migration assay (such as the assay described in Example 3D for pDC's and Example 4F for mDC's) at least 1%, preferably at least 5%, and more preferably at least 10% of the activated pDC's or mDC's cells should migrate in response to a chemoattractant (CCL19 or CCL21);
at least 10%, preferably at least 40%, and most preferably at least 80% of the activated DC's should, in a random migration assay such as the assay of Example 4F below, randomly migrate on fibronectin-coated plates;
in the case of activated pDC's, the activated pDC's (at 1 million pDC's per ml) should be capable of producing at least 100 pg/ml, preferably at least 1000 pg/ml, more preferably at least 5000 pg/ml IFN-alpha (for example determined as described in Example 3C below);
in the case of activated mDC's, the activated mDC's (at 1 million mDC's per ml) should be capable of producing at least 50 pg/ml, preferably at least 100 pg/ml, most preferably at least 500 pg/ml IL-12p70;
the matured and antigen-loaded DC's obtained using the methods described hereon (either pDC's or mDC's) should be capable of inducing the production of IFN gamma by T-cells (at 1 million T cells per ml) with which they are contacted at a level of at least 50 pg/ml, preferably at least 500 pg/ml, more preferably at least 1000 pg/ml (for example determined as described in Example 3C or 41 below);

and such pDC's or mDC's that have been obtained using the methods of the invention and that optionally further have been loaded with one or more tumor antigens (such as those expressed by the tumor(s) to be treated) are particularly suited for use in the immunotherapy of cancer, and form a particularly preferred aspect of the invention.

As mentioned herein, the vaccines used in the methods described herein will generally contain one or more components that are capable of inducing an immune response, and in particular one or more components that are capable of activating (as defined herein) the intended or desired antigen-presenting cell(s) (and in particular, but without limitation, dendritic cells). Accordingly, the term “antigenic component” is generally defined herein as any component or combination of components that is capable of activating antigen-presenting cell(s) (and in particular, but without limitation, dendritic cells). In particular, the vaccines used in the methods described herein may contain any such antigenic component (or combination of components) that is capable of activating antigen-presenting cell(s) (and in particular, but without limitation, dendritic cells) via interaction with (and in particular binding to) one or more receptors that are expressed by the APC's (i.e. expressed on the surface of the APC's/DC's or expressed intracellularly).

More in particular, and although the invention is not limited to a specific hypothesis, mechanism or explanation, it is assumed that the vaccines used in the methods described herein are capable of activating APC's (and in particular DC's) through the interaction of one or more of the antigenic components present in the vaccine with one or more RNA sensors, and in particular one or more dsRNA sensors like PKR, RIG-1, MDA-5 and/or 2,5-OAS and/or one or more “toll-like receptors” or “TLR's” that are expressed by the APC's (and in particular DC's) to be activated (i.e. expressed on the surface of the APC's/DC's or expressed intracellularly). These TLR's may in particular be one or more of the following TLR's: TLR-1, TLR-2, TLR-3, TLR-4, TLR-5, TLR-6, TLR-7, TLR-8, TLR-9, TLR-10, TLR-11, TLR-12 and/or TLR-13, and/or any other TLR's expressed by APC's (and in particular DC's) that are yet to be identified and/or characterized as of the date of filing of the present application. From the further description herein, it will also become clear to the skilled person that these interactions may depend on the specific antigenic component or components that are present in the vaccine, as well as on the TLR's that are present on the APC/DC to be activated (e.g., in the case of DC's, pDC's or mDC's), as different types of APC's/DC's may carry or express different TLR's.

For an overview (non-limiting) of some of the currently identified TLR's expressed by human dendritic cells, their localization, their ligands and their microbial ligands, reference is made to Table 3 below, as well as the further prior art cited herein (note: as of the date of filing of the present application, TLR's 11 to 13 have been identified, but some of their properties have not been characterised in full. Nevertheless, it is envisaged that, based on the disclosure herein, the skilled person will be able to determine, once more detailed information on these TLR's becomes available, whether and how said TLR's can be made use of in the practice of the present invention).

TABLE 3
TLR's expressed by dendritic cells.

| DC | | | | |
|---|---|---|---|---|
| subset | TLR | Localization | Ligand | Microbial ligand |
| MDC | 1 | Cell surface | Lipids & | Triacyl lipopeptides |
| | | | lipopeptides | |
| | 2 | Cell surface | Lipids & | Triacyl lipopeptides |
| | | | lipopeptides | |
| | 3 | Intracellular | Nucleic acids | dsRNA |
| | 4 | Cell surface | Lipids & | LPS |
| | | | lipopeptides | |
| | 5 | Cell surface | Proteins | Flagellin |
| | 6 | Cell surface | Lipids & | Diacyl lipopeptides |
| | | | lipopeptides | |
| | 7 (low) | Intracellular | Nucleic acids | ssRNA (viral) |
| | 8 | Intracellular | Nucleic acids | ssRNA (viral) |
| | 10 | Cell surface | Unknown | Unknown |
| PDC | 1 | Cell surface | Lipids & | Triacyl lipopeptides |
| | | | lipopeptides | |
| | 6 | Cell surface | Lipids & | Diacyl lipopeptides |
| | | | lipopeptides | |
| | 7 (high) | Intracellular | Nucleic acids | ssRNA (viral) |
| | 8 | Intracellular | Nucleic acids | ssRNA (viral) |
| | 9 (high) | Intracellular | Nucleic acids | DNA (bacterial/viral) |
| | 10 | Cell surface | Unknown | Unknown |

As can be seen from Table 3, and as will be explained in more detail below, the pathogen-encoded ligands of TLR's may generally be subdivided into three broad classes, i.e. lipids and lipoproteins (recognized by TLR1/TLR2, TLR6/TLR1 and TLR-4), proteins (TLR-5) and nucleic acids (TLR-3, TLR-7, TLR-8 and TLR-9). The ligands for TLR-10 are currently unknown.

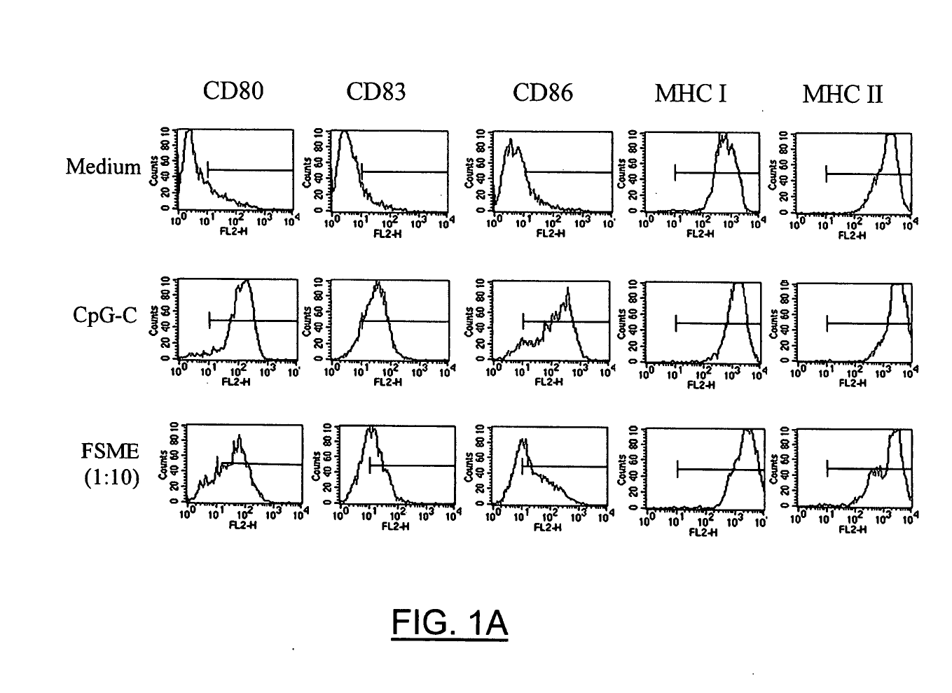

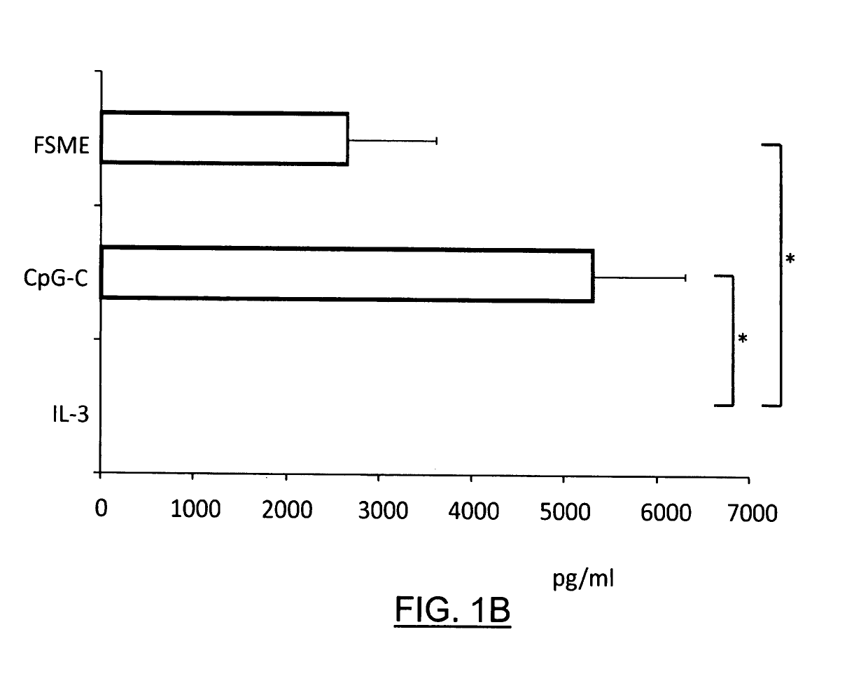

In particular, it has been described in the art (see for example FIG. 1 on page 4 of the review by Takeda and Akira, supra, as well as for example Table 1 of the review by Kanzler, supra) that both TLR-1 and TLR-6 can associate with TLR-2, and when associated recognize triacylated and diacylated lipoprotein, respectively. TLR-3 recognizes (viral) dsRNA, TLR-4 inter alia recognizes LPS and envelope proteins, and TLR-5 recognizes flagellin. TLR-7 and TLR-8 recognize (viral) single stranded RNA and have been implicated in the recognition of small molecule immune response modifiers such as the imidazoqunolines imiquimod and resiquimod. TLR-9 recognizes (bacterial or viral) DNA and has been implicated in the recognition of CpG oligonucleotides (which are also used as TLR ligands).

It has also been reported that, whereas myeloid DCs express most TLRs known to date except TLR7 and 9, pDCs have a very distinctive expression of TLRs. They express high levels of TLR7 and 9 and moderate levels of TLR 1, 6, and 10; and they do not express TLR-2, TLR-3, TLR-4 and TLR-5, and therefore do not respond to bacterial components such as peptidoglycans, LPS or flagellin, nor to extracellular double-stranded RNA, but solely recognize DNA and RNA viruses (i.e. via TLR-7, TLR-8 and TLR-9, which are expressed by pDC's). Reference is for example made to Barchet et al., supra, page 3, and the further references cited therein.

It is also mentioned by Barchet et al. that TLR-9 is engaged by unmethylated CpG rich DNA that is common in bacteria and the genomes of DNA viruses, whereas TLR-7 mediates the recognition of ribonucleotide homologs such as loxoribine, of single stranded RNA sequences and of single stranded RNA viruses, such as Influenza virus and vesiculostomatitis (VSV) virus.

Barchet et al. and Kanzler et al. also suggest that the interaction of the viruses, viral particles or viral products with the TLR's expressed by pDC's only takes place upon endocytosis of the viruses, viral particles or viral products by the pDC's, This is because the TLR's that recognize viruses (i.e. viral nucleic acids), such as TLR-3 (which is expressed by mDC's) and TLR-7, TLR-8 and TLR-9 (which are expressed by mDC's and pDC's), are expressed intracellularly and confined to an acidic endosomal compartment (unlike for example the TLR's present on mDC's that are involved in the recognition of bacterial products such as TLR-1/TLR-2, TLR-6/TLR-2 and TLR-5, which TLR's are expressed on the surface of the mDC's).

Thus, according to a specific but non-limiting aspect of the invention, the vaccine used in the methods described herein is a vaccine that contains one or more antigenic components (as defined herein) that are capable of activating (as defined herein) APC's (and in particular DC's) via interaction with one or more TLR's that are expressed by the APC's (and in particular DC's). Generally, this may be any suitable vaccine that contains one or more (microbial) ligands of one or more of the TLR's that are expressed by the APC's (and in particular DC's) to be activated, and/or any vaccine that contains a (weakened, attenuated or inactivated) pathogen that contains, expresses or encodes such a (microbial) ligand. Reference is generally made to Table 1 and the further disclosure herein.

For example, such a vaccine may contain inactivated, weakened or attenuated bacteria or viruses; inactivated, weakened or attenuated viral particles; nucleic acids (DNA, single stranded RNA or double stranded RNA) that are contained in or encoded by bacteria or viruses (or from another suitable micro-organism); or alternatively any other suitable antigenic components that are based on (and/or that have been derived from) such micro-organisms, such as bacterial or viral proteins (for example cell wall proteins, viral coat proteins, envelope proteins or other suitable bacterial or viral antigens, or any suitable fragment of the foregoing antigens; these may optionally also be suitably conjugated, for example with tetanus toxoid), as well as cell fragments or cell fractions that have been derived from bacteria, viruses or other suitable micro-organisms. Specific examples of such vaccines and antigenic components will be clear to the skilled person based on the disclosure herein, and for example include (without limitation) the commercially available vaccines referred to herein (and/or the antigenic components present therein), as well as the vaccines mentioned in Tables 2-5 of the review by Kanzler mentioned above (and/or the antigenic components present therein).

Generally, in the invention, the use of vaccines that contain bacteria and/or (inactivated, weakened or attenuated) viruses, virus particles or virus-derived antigenic components that are capable of activating APC's (and in particular DC's) via interaction with one or more TLR's will be preferred (in particular for activating pDC's, as will be further discussed below). In particular, such vaccines may contain (inactivated, weakened or attenuated) viruses or virus particles that contain or encode nucleic acids (i.e. DNA, single stranded RNA or double stranded RNA) that can that interact with TLR's expressed by the DC's that recognize such nucleic acids and/or that have such nucleic acids as a ligand (such as TLR-3, TLR-7, TLR-8 and/or TLR-9). For example, such vaccines may contain (inactivated, weakened or attenuated) DNA viruses, double stranded RNA viruses or single stranded RNA viruses; and in particular DNA viruses or single stranded RNA viruses, such as influenza virus or flaviviruses such as yellow fever virus and tick-borne encephalitis virus. Alternatively, as mentioned herein, vaccines may be used that contain nucleic acids contained in or encoded by such viruses (i.e. viral DNA, single stranded RNA or double stranded RNA).

In one preferred, but non-limiting aspect, the vaccine used in the methods described herein is such that its ability to activate pDC's (as described herein) is inhibited or reduced when the pDC's is simultaneously incubated with both the vaccine as well as an inhibitor of endosomal maturation (such as chloroquine).

In another preferred, but non-limiting aspect, the vaccine used in the methods described herein is such that its ability to activate pDC's (as described herein) is inhibited or reduced when the pDC's is simultaneously incubated with both the vaccine as well as an antagonist of a TLR, in particular an antagonist of an endosomal TLR (such as TLR-7 or TLR-9), and more in particular an inhibitor of TLR-9.

In the practice of the invention, particularly good results have been obtained with vaccines containing (inactivated, weakened or attenuated) FSME, a tick-borne encephalitis virus, such as FSME-Immun™. As described herein, FSME is particularly suited for the activation of pDC's using the methods described herein, and when used in such methods is capable of both inducing increased production of IFN-alpha as well as inducing the maturation of pDC's. Alternatively, (a composition comprising) one or more suitable antigenic components derived therefrom (such as nucleic acids) may be used. Table 4 in Example 6 below shows the binding of some of the vaccines that can be used in the practice of the invention to different TLR's.

Based on the disclosure herein, it will be clear to the skilled person that the specific vaccine to be used in the methods described herein may also depend on the specific APC's (and in particular DC's, i.e. pDC's or mDC's), and may in particular depend on the specific TLR's that are expressed by the APC's to be activated. For example, and without limitation, when the methods described herein are to be used to activate pDC's, preferably a vaccine is used that is capable of activating the pDC's by interaction with one or more of the following TLR's: TLR-1, TLR-6, TLR-7, TLR-8, TLR-9 and TLR-10; and in particular TLR-7, TLR-8 and/or TLR-9; and/or that contains one or more antigenic components that are capable of activating the pDC's by interaction with one or more of the following TLR's: TLR-1, TLR-6, TLR-7, TLR-8, TLR-9 and TLR-10; and in particular TLR-7, TLR-8 and/or TLR-9, preferably TLR-7 or TLR-9, and most preferably (at least) TLR-9. As mentioned above, such a vaccine may in particular contain a weakened, attenuated or inactivated virus or viral particle that is capable of activating pDC's via interaction with TLR-7, TLR-8 and/or TLR-9; and/or contain a nucleic acid (DNA, single stranded RNA or double stranded RNA) that is capable of activating pDC's via interaction with TLR-7, TLR-8 and/or TLR-9 (or a virus or viral particle that contains or encodes such a nucleic acid).

For example, such viruses may be DNA viruses, double stranded RNA or single stranded RNA viruses, and in particular DNA viruses or single stranded RNA viruses, such as influenza virus or flaviviruses such as yellow fever virus and tick-borne encephalitis virus (and consequently, the nucleic acids present in such vaccines or contained in or encoded by said viruses may be DNA, single stranded RNA or double stranded RNA). In particular, a vaccine may be used that contains (inactivated, weakened or attenuated) FSME (such as FSME-Immun™) or a nucleic acid derived from or encoded by FSME.

When the methods described herein are to be used to activate mDC's, preferably a vaccine is used that is capable of activating the mDC's by interaction with one or more of the following TLR's: TLR-1, TLR-2, TLR-3, TLR-4, TLR-5, TLR-6, TLR-7, TLR-8 and TLR-10, and in particular TLR-3, TLR-7 or TLR-8; and/or that contains one or more antigenic components that are capable of activating the mDC's by interaction with one or more of the following TLR's: TLR-1, TLR-2, TLR-3, TLR-4, TLR-5, TLR-6, TLR-7, TLR-8 and TLR-10; and in particular with TLR-2, TLR-3, TLR-4, TLR-5, TLR-7 and/or TLR-8, and most preferably with TLR-2, TLR-4 and/or TLR-5. As mentioned above, such a vaccine may in particular contain a weakened, attenuated or inactivated virus or viral particle that is capable of activating mDC's via interaction with TLR-3, TLR-7 and/or TLR-8; and/or contain a nucleic acid (DNA, single stranded RNA or double stranded RNA) that is capable of activating mDC's via interaction with TLR-3, TLR-7 and/or TLR-8 (or a virus or viral particle that contains or encodes such a nucleic acid).

According to another specific aspect, a vaccine may be used that contains one or more antigenic components that are can be electroporated into, endocytosed by, or otherwise taken up by and/or incorporated into APC's (and in particular DC's) and that, upon such uptake, are capable of activating the APC's, in particular via interaction with one or more TLR's that are expressed intracellularly by the APC's. Without being limited to a specific explanation, mechanism or hypothesis, in the case of pDC's, this may for example be a vaccine that contains one or more antigenic components that can be endocytosed by pDC's and that, upon such endocytosis, are capable of activating the pDC's by interaction with one or more of TLR's that are expressed intracellularly by pDC's, and in particular with one or more of the following TLR's: TLR-7, TLR-8 and/or TLR-9. In the case of mDC's, this can for example be a vaccine that contains one or more antigenic components that can be electroporated into or endocytosed by mDC's and that, upon such uptake, are capable of activating the mDC's by interaction with one or more TLR's that are expressed intracellularly by mDC's, and in particular with one or more of the following TLR's: TLR-2, TLR-3, TLR-4, TLR-5, TLR-7 or TLR-8, and preferably TLR-2, TLR-4 and/or TLR-5. Again, this may be a vaccine that contains one or more bacteria or viruses, virus particles or other viral-derived antigenic components (including nucleic acids) that can be electroporated into or endocytosed by antigen presenting cells such as pDC's and/or mDC's, and that contain or encode nucleic acids that are recognized by one or more TLR's that are intracellularly expressed by the DC's (such as one or more of the TLR's mentioned above).

As described above, vaccines that are suitable for use in the methods described herein and that contain one or more of the aforementioned antigenic components may be in any suitable form, such as in the form of a formulation or preparation (as described herein), which may be a ready-to-use formulation or preparation (or in a form that can be constituted into a ready-to-use form) and/or a commercial formulation or preparation. Again, such formulations and preparations are preferably approved for use in or in connection with human subjects.

Also, again, vaccines for use in the methods described herein may be contained in a suitable container (such as a flask, vial, bag or syringe) that may be packaged together with instructions for use of the vaccine in the methods described herein (or more generally, for use of the vaccine in methods for activating and optionally loading dendritic cells) or with a product information leaflet.

A vaccine for use in the methods described herein may also be provided as part of a kit-of-parts, as further described herein.

It will also be clear to the skilled person that - instead of a suitable (formulated) vaccine as mentioned herein - it is also possible to use, in the methods described herein, one or more of the antigenic components that are present in such a vaccine. Furthermore, it is also possible to use a suitable composition comprising such antigenic component(s), as further described herein.

Such antigenic components may in particular be as described herein, and may for example be one of the microbial ligands for TLR's mentioned above and/or one of the other suitable antigenic components mentioned above, such as one or more suitable antigenic components that are present in one of the vaccines mentioned herein. The antigenic component(s) may also be, again without limitation, a bacterium, virus, viral particle, nucleic acid that is derived from a bacterium or virus, or any other suitable composition or preparation that can be (or has been) derived from a bacterium or virus (such as a bacterial or viral lysate, fragment, fraction, supernatant or suspension); provided the foregoing are capable of activating APC's (and in particular DC's) as described herein.

For example, for activating pDC's, again one or more antigenic components may be used that are capable of activating the pDC's by interaction with one or more of the following TLR's: TLR-1, TLR-6, TLR-7, TLR-8, TLR-9 and TLR-10; and in particular TLR-7, TLR-8 and/or TLR-9, preferably TLR-7 or TLR-9, and most preferably (at least) TLR-9. This may again be a weakened, attenuated or inactivated virus or viral particle that is capable of activating pDC's via interaction with TLR-7, TLR-8 and/or TLR-9; and/or a nucleic acid (DNA, single stranded RNA or double stranded RNA) that is capable of activating pDC's via interaction with TLR-7, TLR-8 and/or TLR-9 (or a virus or viral particle that contains or encodes such a nucleic acid). Specific examples thereof may be as mentioned above.

Similarly, for activating mDC's, one or more antigenic components may be used that are capable of activating the mDC's by interaction with one or more of the following TLR's: TLR-1, TLR-2, TLR-3, TLR-4, TLR-5, TLR-6, TLR-7, TLR-8 and/or TLR-10, and in particular with TLR-2, TLR-3, TLR-4, TLR-5, TLR-7 and/or TLR-8, and most preferably with TLR-2, TLR-4 and/or TLR-5. These may also be a weakened, attenuated or inactivated virus or viral particle that is capable of activating mDC's via interaction with TLR-3, TLR-7 and/or TLR-8; and/or a nucleic acid (DNA, single stranded RNA or double stranded RNA) that is capable of activating mDC's via interaction with TLR-3, TLR-7 and/or TLR-8 (or a virus or viral particle that contains or encodes such a nucleic acid). Specific examples thereof may be as mentioned above.

Again, according to one specific but non-limiting aspect, such antigenic components may be antigenic components that can be electroporated into or endocytosed by antigen presenting cells such as DC's (i.e. by pDC's and/or by mDC's, respectively, as described herein) and/or antigenic components that are capable of activating DC's via interaction with TLR's that are expressed intracellularly by the DC's (i.e. by pDC's and/or by mDC's, respectively, as described herein).

Thus, in another aspect, the invention relates to a method for providing a composition that comprises at least one activated (as defined herein) antigen-presenting cell (and in particular, but without limitation, dendritic cell), which method at least comprises the step of:
a) providing a composition that comprises at least one antigen-presenting cell;
b) contacting said composition with one or more antigenic components (as defined herein) that are capable of activating (as defined herein) said antigen-presenting cell (and/or with a composition or preparation that comprises one or more such antigenic components).

Again, in this method, the antigen-presenting cell may be any desired or intended antigen-presenting cell, but may in particular be a dendritic cell (as further described herein).

The invention also relates to a composition that comprises at least one antigen-presenting cell (and in particular, but without limitation, dendritic cell) that has been activated (as defined herein) using one or more antigenic components (as described herein; and optionally in the form of a suitable composition, also as described herein) and/or using one of the methods described herein.

The invention further relates to an antigen-presenting cell (and in particular, but without limitation, dendritic cell) that has been activated using one or more antigenic components (as described herein; and optionally in the form of a suitable composition, also as described herein) and/or using one of the methods described herein, and to compositions comprising at least one such activated antigen-presenting cell.

The invention further relates to the use of an antigenic component (as described herein; and optionally in the form of a suitable composition, also as described herein) in the preparation of a composition that comprises at least one activated antigen-presenting cell (and in particular, but without limitation, dendritic cell), and also to the use of an antigenic component (as described herein; and optionally in the form of a suitable composition, also as described herein) in activating an antigen-presenting cell (and in particular, but without limitation, dendritic cell).

The invention further relates to an antigenic component (as defined herein) for use in activating antigen-presenting cells (and in particular, but without limitation, dendritic cells), and to the use of an antigenic component (as defined herein) in the preparation of a composition for activating antigen-presenting cells (and in particular, but without limitation, dendritic cells). The invention also relates to a composition comprising one or more such antigenic components for activating antigen-presenting cells (and in particular, but without limitation, dendritic cells).

The invention also relates to a method for activating (as defined herein) an antigen-presenting cell (and in particular, but without limitation, dendritic cell), which method comprises contacting the antigen-presenting cell with one or more antigenic components (as defined herein), wherein the contacting of the antigen-presenting cell with the antigenic component(s) is performed by contacting a composition that comprises the antigen-presenting cell with a vaccine or other composition or preparation that comprises the antigenic component(s).

In yet another aspect, the invention relates to a method for providing a composition that comprises an activated antigen-presenting cell (and in particular, but without limitation, dendritic cell) that has been loaded with one or more desired antigens, which method comprises at least the steps of:
a) providing a composition that comprises at least one antigen-presenting cell;
b) contacting said composition with one or more antigenic components (as defined herein) that are capable of activating (as defined herein) said antigen-presenting cell (and/or with a composition or preparation that comprises such an antigenic component); and
c) loading (as defined herein) the activated antigen-presenting cell with the one or more desired antigens.

Again, in this method, the antigen-presenting cell may be any desired or intended antigen-presenting cell, but may in particular be a dendritic cell (as further described herein).

In the above method, after step b) and before step c), (the composition comprising) the APC's/DC's may be treated or washed in order to remove the antigenic component and the activating composition (or any excess thereof).

Also, as described herein, when a virus, viral particle, viral nucleic acid, viral vector or other virus-derived composition or preparation is used as the antigenic component, such a virus-derived antigenic component may further be such that it is capable of loading the APC's/DC's with one or more desired antigens. For this purpose, the virus-derived antigenic component may for example encodes the desired antigen(s) and/or contain or comprise a nucleic acid that encodes the desired antigen(s), and may further be such that is capable of transforming or transfecting the APC's/DC's with a nucleic acid encoding the desired antigen(s), such that the APC's/DC's express the desired antigen(s). For example, a suitable gene therapy vector that is derived from a virus or based on a viral nucleic acid and that encodes the antigen(s) may be used.

The invention also relates to a composition that comprises at least one antigen-presenting cell (and in particular, but without limitation, dendritic cell) that has been activated (as defined herein) using one or more antigenic components (as described herein; and optionally in the form of a suitable composition, also as described herein) and loaded (as defined herein) with one or more desired antigens using the above method.

The invention further relates to an antigen-presenting cell (and in particular, but without limitation, dendritic cell) that has been activated (as defined herein) using one or more antigenic components (as described herein; and optionally in the form of a suitable composition, also as described herein) and loaded (as defined herein) with one or more desired antigens using the above methods, and to compositions comprising at least one such activated and loaded antigen-presenting cell.

The invention further relates to the use of an antigenic component in the preparation of a composition that comprises at least one activated and loaded antigen-presenting cell (and in particular, but without limitation, dendritic cell), and also to the use of an antigenic component in preparing such an activated and loaded antigen-presenting cell.

The invention also relates to an antigenic component for preparing activated and loaded antigen-presenting cells (and in particular, but without limitation, dendritic cells).

The invention further relates to applications and uses of an antigen-presenting cell (and in particular, but without limitation, dendritic cell) that has been activated and loaded using the above method (and to uses of compositions comprising such an activated and loaded antigen-presenting cell). Such applications and uses may again be as further described herein.

Again, the antigenic components used in the methods described herein are preferably safe for use in or in connection with human subjects; and/or may be antigenic components that are part of (and/or used in the preparation of) vaccines that have been approved for use in human subjects.

In one preferred, but non-limiting aspect, one or more antigenic components (or mixture thereof) used in the methods described herein are such that, when these antigenic components are contacted with the DC's to be activated, they are capable of increasing the production by the DC's of cytokines that are usually produced by such (activated) DC's (such as Type I interferons and in particular of IFN-alpha in the case of pDC's, and IL-12p70 in the case of mDC's), i.e. by at least 1%, preferably by at least 10%, such as by at least 20%, for example by 50% or more, compared to the DC's before they are contacted with the vaccine. This may for example be determined as described in the Experimental Section below. This aspect of the invention has been found to be particularly suited for the activation of pDC's, but can also be used for the activation of mDC's. Examples of such antigenic components will be clear to the skilled person based on the disclosure herein, and may for example be derived from vaccines that are capable of increasing the production of Type I interferons. In one specific aspect, the antigenic component(s) or mixture of antigenic components used is capable of increasing the production of Type I interferons without substantially inducing the maturation of the DC's.

In another preferred, but non-limiting aspect, the one or more antigenic components (or mixture thereof) used in the methods described herein are such that, when these antigenic components are contacted with the DC's to be activated, they are capable of inducing the maturation of pre-DC's into DC's (and in particular, into pDC's), as measured by the upregulation (i.e. increased expression) of the costimulatory molecules CD80, CD83 and/or CD86 and increased expression of the antigen presenting molecules MHC class I and MHC class II by the DC's (i.e. by at least 1%, preferably by at least 5%, such as by at least 10%, for example by 25% or more, compared to the DC's before they are contacted with the vaccine). Again, this may for example be determined as described in the Experimental Section below. This aspect of the invention has been found to be particularly suited for the activation of pDC's, but can also be used for the activation of mDC's. Examples of such antigenic components will be clear to the skilled person based on the disclosure herein, and may for example be derived from vaccines that are capable of inducing pDC maturation. In one specific aspect, the antigenic component(s) or mixture of antigenic components used is capable of inducing the maturation of the DC's without substantially increasing the production of Type I interferons.

In yet another preferred, but non-limiting aspect, the one or more antigenic components (or mixture thereof) used in the methods described herein are such that, when these antigenic components are contacted with the DC's to be activated, they are capable of both increasing the production by the DC's of cytokines that are usually produced by such (activated) DC's (such as Type I interferons and in particular of IFN-alpha in the case of pDC's, and IL-12p70 in the case of mDC's), i.e. by at least 1%, preferably by at least 10%, such as by at least 20%, for example by 50% or more, compared to the DC's before they are contacted with the vaccine, as well as inducing the maturation of pre-DC's into DC's (and in particular, into pDC's), as measured by the upregulation (i.e. increased expression) of the costimulatory molecules CD80 and/or CD86 and increased expression of the antigen presenting molecules MHC class I and MHC class II by the DC's (i.e. by at least 1%, preferably by at least 5%, such as by at least 10%, for example by 25% or more, compared to the DC's before they are contacted with the vaccine). Again, this may for example be determined as described in the Experimental Section below. This aspect of the invention has been found to be particularly suited for the activation of pDC's, but can also be used for the activation of mDC's. Examples of such antigenic components will be clear to the skilled person based on the disclosure herein, and include antigenic components that are derived from vaccines that are capable of both increasing production of Type interferons as well as inducing pDC maturation (such as FSME). Also, in the practice of the present invention, the use of antigenic components that are capable of both increasing IFN Type I production as well as inducing pDC maturation will usually be preferred, although the invention is not limited thereto.

It should also be noted that it is possible in the invention to activate DC's by using two or more different antigenic components, and that in doing so, a synergistic effect may be obtained. For example, when two or more different antigenic components are used, at least one antigenic component may be used that is capable of increasing the production of Type I interferons such as IFN-alpha, and at least one other antigenic component may be used that is capable of inducing DC maturation. Other combinations of suitable antigenic components may also be used. When DC's are activated according to the methods described herein using two or more different vaccines, the DC's to be activated may be contacted with a mixture of the two or more different antigenic components, may be contacted simultaneously with the two or or more different antigenic components, or may be contacted with the two or more different antigenic components in two or more separate steps (usually performed shortly after one another).

It is also possible to use, in addition to the antigenic component(s) or combination or mixture of antigenic components used, to use one or more antigenic components as described herein in combination with one or more cytokines (such as TNF-alpha, IL-6 and/or IL-lbeta, and/or other pharmaceutically acceptable cytokines that have been used in the art to stimulate pDC's or mDC's, respectively) and/or one or more suitable hormones such as prostaglandins (for example Prostaglandin E2). These may be mixed with the antigenic component (s) used, or the antigenic component(s) and the cytokines and/or hormones may be contacted with the DC's to be activated simultaneously or in separate steps (usually performed shortly after one another).

In addition, the antigenic vaccine component(s) (or mixture or combination thereof used) are preferably such that, and the methods described herein are preferably performed such that:
a) the resulting DC's are have the ability (or an improved ability) to migrate from the injection site to T cell areas in lymph nodes where they can then present the antigen to T cells, as may for example be determined by measuring the kinetics of acquisition of migratory function (for example using the chemotaxis assay or, in the case of mDC's, the random migration assay described in the Experimental Part below). This ability to migrate is preferably such that the resulting DC's are suitable for use in cancer immunotherapy. The migratory capacity of the DC's obtained using the methods of the invention may further be increased by adding a prostaglandine such as PGE₂;
   and/or
b) the resulting DC's are have the ability (or an improved ability) to produce the cytokines that are usually produced by such (activated) DC's (such as Type I interferons and in particular of IFN-alpha in the case of pDC's, and IL-12p70 in the case of mDC's), as may for example be determined using the cytokine detection assays like ELISA's or cytokine detection bead assays described in the Experimental Part below. This ability to produce Type I IFN is preferably such that the resulting DC's are suitable for use in cancer immunotherapy;
   and/or
c) the resulting DC's are have the ability (or an improved ability) to induce Th1-type CD4+ T cells and CD8+ cytotoxic T lymphocytes, as may for example be determined using T cell stimulation assays (i.e. primary inductions, mixed lymphocyte reaction, stimulation of antigen specific T cell lines) described in the Experimental Part below. This ability to induce of Th1-type CD4+ T cells and CD8+ cytotoxic T lymphocytes is preferably such that the resulting DC's are suitable for use in cancer immunotherapy;
   and/or
d) the resulting DC's are have the ability (or an improved ability) to express co-stimulatory molecules such as [CD80 and CD86] and have the ability (or an improved ability) to express of the antigen presenting molecules MHC class I and MHC class II, as may for example be determined using the flow cytometric assays described in the Experimental Part below. This ability to express co-stimulatory molecules is preferably such that the resulting DC's are suitable for use in cancer immunotherapy;
   and/or
e) the resulting DC's are have the ability (or an improved ability) to induce a Th1 response, as may for example be determined using the cytokine bead or cytokine ELISA assays described in the Experimental Part below. This ability to induce a Th1 response is preferably such that the resulting DC's are suitable for use in cancer immunotherapy.

Again, preferably DC's are obtained (i.e. either pDC's or mDC's, and activated using one or more vaccines and/or one or more antigenic components derived therefrom) that have the preferred properties described above, and that thus are particularly suited for the immunotherapy of cancer (optionally after loading with one or more tumor antigens or a mixture thereof).

In one specific, but non-limiting aspect, the antigenic component(s) or mixture combination of antigenic components that is used in the methods described herein is such that its ability to activate pDC's (as described herein) is inhibited or reduced when the pDC's is simultaneously incubated with an inhibitor of endosomal maturation (such as chloroquine).

In another preferred, but non-limiting aspect, the antigenic component(s) or mixture or combination of antigenic components that is used in the methods described herein is such that its ability to activate pDC's (as described herein) is inhibited or reduced when the pDC's is simultaneously incubated with an antagonist of a TLR, in particular an antagonist of an endosomal TLR (such as TLR-7 or TLR-9), and more in particular an inhibitor of TLR-9.

In one specific, but non-limiting aspect, the one or more antigenic components for use in the methods described herein may be contained in, part of, and/or used in the form of a suitable formulation or preparation, such as a solution or suspension of such antigenic components in a suitable medium, such as water, a physiologically acceptable (usually aqueous) buffer or solution or another suitable (aqueous) medium that is suitable for administration to a subject. Such a formulation or preparation may, in addition to the one or more antigenic components, contain one or more suitable constituents or carriers for such compositions known per se. Such a composition or formulation may also be in a form that is ready for its intended use (or in a form that can be constituted into a ready-to-use form).

Also, the antigenic components for use in the methods described herein (or a composition or formulation thereof) may be contained in a suitable container (such as a flask, vial, bag or syringe) that may be packaged together with instructions for use of the antigenic components (or composition or formulation) in the methods described herein (or more generally, for use of the antigenic components in methods for activating and optionally loading dendritic cells), or with a product information leaflet.

The antigenic component(s) for use in the methods described herein (or a composition or formulation thereof) may also be provided as part of a kit-of-parts, as further described herein.

In the methods described herein, the APC's/DC's may be obtained, handled, cultivated and optionally stored (i.e. prior to use in the methods described herein) in any suitable manner known per se. Suitable methods and techniques will be clear to the skilled person, and for example include the CliniMACS™ procedure, which leads to the development of clinical applicable pDCs having immune stimulatory characteristics. Reference is for example made to the handbooks and prior art mentioned herein. As mentioned herein, according to a specific but non-limiting aspect, when the APC's/DC's are intended for administration to a human subject, they may be obtained from said subject or obtained starting from APC's/DC's that have been obtained from said subject (i.e. by cultivation). For example, when DC's are used, such DC's may be obtained from a subject as DC's (i.e. pDC's) that need to be further activated (as defined herein) using the methods described herein.

Also, the activating of the APC's/DC's (i.e. using a vaccine or one or more antigenic components, as described herein) and the loading of the APC's/DC's (i.e. with the one or more desired antigens), may be performed using techniques for activating and loading APC's/DC's known per se to the skilled person (but using a vaccine or one or more antigenic components to activate the APC's/DC's, as described herein).

Generally, for activating the APC's/DC's, the APC's/DC's may be suitably contacted with the vaccine or with the one or more antigenic components (or a composition comprising the same), under conditions that are such, and in a manner that is such, that the APC's/DC's are activated. This will usually be performed while the APC's/DC's are suspended in a suitable medium, such as a physiological solution or buffer, or another suitable (usually aqueous) medium.

For example, and without limitation, when DC's are used, for activating a sample of between 1 million and 50 million DC's in between 0.2 ml and 1 ml of a physiologically acceptable (aqueous) buffer, solution or medium, the DC's may be contacted with between 0.01 μg/ml and 0.5 μg/ml of the vaccine (for example, an FSME-vaccine as mentioned herein), during a time of between 1 hour and 48 hours and at a temperature of between 20° C. and 37° C. This may for example be performed by simply mixing the sample of the dendritic cells with a vaccine that contains the virus or viral particles. Similar or equivalent conditions may be used for activating other APC's.

After the APC's/DC's have been activated, the sample of activated APC's/DC's may be washed or treated in order to remove the antigenic component and the activating composition (or any excess thereof). This may be performed in any suitable manner known per se, for example by washing with a physiologically acceptable solution, buffer or medium.

The activated APC's/DC's may then be loaded with the one or more desired antigens. This may generally be performed by contacting the activated APC's/DC's with the antigen(s) under conditions that are such, and in a manner that is such, that the APC's/DC's are loaded with the antigen.

For example, and without limitation, when DC's are used, for loading a sample of between 1 million and 50 million activated DC's in between 0.2 ml and 1 ml of water or a physiologically acceptable buffer, solution or medium, the DC's may be contacted with between 1 μM and 50 μM tumor-derived 9-mer peptides (units of the antigen(s)), during a time of between 1 hour and 4 hours and at a temperature of between 20° C. and 37° C. This may for example be performed by simply mixing the sample or suspension of the dendritic cells with a suspension or solution of the antigen(s), for example in a physiologically acceptable (aqueous) buffer, solution or medium. Similar or equivalent conditions may be used for activating other APC's.

The activated and loaded APC's/DC's may then optionally be washed in order to remove excess of activating composition and antigen(s), whereupon the activated and loaded APC's/DC's will usually be ready for use.

Generally, in the methods described herein, the APC's/DC's will be activated and loaded immediately prior to use. However, it is also possible to suitably store either the activated APC's/DC's (which may then be loaded immediately prior to use) or the activated and loaded APC's/DC's prior to use. Suitable techniques for storing (activated or activated/loaded) DC's will be clear to the skilled person, and for example include freezing in DMSO-containing media below −80° C. (see for example Feuerstein et al., Journal of Immunological Methods, 245 (2000), 15-29) or other suitable (cryo)preservation techniques known per se to the skilled person. Similar or equivalent techniques may be used for storing other APC's.

In the methods described herein, the APC's/DC's may be loaded with any desired antigen or antigens. The antigen will usually be a protein, (poly)peptide or other ligand that can be presented by APC's (and in particular DC's) to (other) cells of the immune system, such as B-cells and in particular T-cells, but may for example also be a suitable nucleic acid, and/or may be in the form of a suitable composition or preparation (for example, and without limitation, a cell fragment, cell extract, cell fraction or cell lysate, derived from the cell against which the immune response is to be raised or from a cell or cell line that contains or carries one or more antigenic determinants that are essentially the same as those expressed by the cell against which the immune response is to be raised). In particular, the antigen may be any protein, (poly)peptide or other ligand that can bind to (one or more receptors on) the surface of the APC's/DC's (and in particular to the MHC on the surface of the APC's/DC's) and/or that can be expressed on the surface of the APC's/DC's (i.e. following transient transformation or transfection of the APC/DC with a nucleic acid encoding the same), as further described herein.

When the APC's/DC's are loaded with a nucleic acid (for example with DNA, single stranded RNA or double stranded RNA), such nucleic acid may for example encode the relevant antigen. Also, when the APC's are to be loaded with RNA, preferably (single stranded) RNA such as mRNA is used, to prevent any RNA interference that might occur if double stranded RNA is used.

As will be clear to the skilled person, the choice of the antigen(s) will usually depend upon the intended use of the activated and loaded APC's/DC's. As mentioned herein, the activated and loaded APC's/DC's can generally be used for presenting the antigen(s) to T-cells in order to elicit an antigen-specific immune response (e.g. B-cell or T-cell mediated) against said antigen(s), either in vivo (e.g. for immunotherapy in a subject to be treated) or ex vivo (e.g. in a suitable cellular assay system or model system). In such a case, the choice of the antigen(s) will generally depend on the desired antigen-specific response to be obtained.

For example, by administering the activated and loaded APC's/DC's to a human subject, it is possible to trigger a T-cell mediated cytotoxic response or other specific (immune) response against the antigen(s) in said subject. As will be clear to the skilled person, this makes the activated and loaded APC's/DC's suitable for methods of immunotherapy in a subject, which methods at least comprise administration of the activated and loaded APC's/DC's (or of a suitable composition comprising the same) to a subject in need thereof Again, in this aspect, the choice of the antigen(s) will generally depend on the desired antigen-specific immune response to be obtained, which in turn will depend on the disease or condition to be prevented or treated in said subject.

Thus, in another aspect, the invention relates to a composition for immunotherapy in a subject, which composition comprises an antigen-presenting cell (and in particular, but without limitation, dendritic cell) that has been activated and loaded using the methods described herein, wherein said antigen-presenting cell has been loaded with an antigen that is suitable for (and/or intended for) use in immunotherapy in said subject.

In another aspect, the invention relates to a composition for generating a (T-cell or B-cell mediated) immune response in a subject, which composition comprises an antigen-presenting cell (and in particular, but without limitation, dendritic cell) that has been activated and loaded using the methods described herein, wherein said antigen-presenting cell has been loaded with the antigen against which the immune response is to be generated in said subject. The composition may in particular be used to generate a specific cytotoxic response against the antigen in said subject, and/or against cells that express the antigen or contain the antigen on their surface.

The above compositions are preferably such that they are suitable for administration to the subject to be treated. As such, they preferably contain APC's/DC's that are suitable for administration to the subject to be treated. In particular, these are preferably APC's/DC's that have been harvested or otherwise obtained from the subject to be treated, and/or APC's/DC's that have been obtained from APC's/DC's that have been harvested or otherwise obtained from the subject to be treated. In addition, the APC's/DC's are preferably loaded with antigens that are suitable for administration to the subject to be treated, and the composition preferably comprises—besides the activated and loaded APC's/DC's—further components and carriers that are suitable for administration to the subject to be treated.

Usually, also, in the above methods, a preparation or sample of APC's/DC's is used that contains a population of APC's/DC's, and in particular a population of APC's/DC's that comprises an amount of APC's/DC's that is suitable for immunotherapy in a subject. For example, and without limitation, when DC's are used, the methods described herein may be used to provide a population of between 100,000 and 100 million DC's, often between 1 million and 50 million DC's, for example in about between 0.2 and 1 ml of a physiologically acceptable buffer or solution. Similar or equivalent amounts may be used when using other APC's. Such a preparation or sample may then be administered to the subject to be treated, for example by means of injection or any other suitable technique for administering APC's/DC's known per se. This is preferably performed according to an administration regimen or dosing schedule that is such that an immune response against the antigen(s) is raised, and may for example, when DC's are used, comprise a single administration of between 1 and 50 million DC's, or several administrations of between 1 and 50 million DC's per administered dose, for example separated by several days. Similar or equivalent amounts may be used when using other APC's. A dosing schedule may also comprise an initial administration/immunization with the APC's/DC's, followed by one or more booster immunizations (optionally combined with administration of other active principles that may for example be intended to boost the immune response or immune system). For example, and without limitation, a suitable regimen may comprise about 3 or 4 such doses distributed between 10 and 28 days, depending on the condition to be prevented or treated and/or on (the strength of) the immune response to be raised. Generally, the clinician will be able to select (and where necessary suitably modify) a suitable treatment regimen for a specific subject and condition to be treated, optionally by suitably monitoring the immune response upon administration of the APC's/DC's. Reference is for example made to the review by Tuyaerts et al. cited herein.

In another aspect, the invention relates to a method for immunotherapy in a subject in need of such immunotherapy, which method at least comprises the step of administering to said subject a preparation or sample of activated and loaded antigen-presenting cells (and in particular, but without limitation, dendritic cells) as described herein.

The invention also relates to a method for generating an immune response in a subject, which method at least comprises the step of administering to said subject a preparation or sample of activated and loaded antigen-presenting cells (and in particular, but without limitation, dendritic cells), wherein said antigen-presenting cells have been loaded with the antigen(s) against which the immune response is to be raised.

The invention further relates to a method for providing an antigen-presenting cell (and in particular, but without limitation, dendritic cell), or preparation or sample of APC's/DC's, for use in immunotherapy in a subject, which method at least comprises the steps of:
a) harvesting a sample or population of antigen-presenting cells from said subject (and in particular, of DC's, and more in particular, pDC's);
b) activating the antigen-presenting cells in said sample or population using the methods described herein; and
c) loading the antigen-presenting cells with one or more antigens that are suitable for (and/or intended for) immunotherapy in said subject.

Again, in this method, the antigen-presenting cell may be any desired or intended antigen-presenting cell, but may in particular be a dendritic cell (as further described herein).

The invention further relates to a method for immunotherapy in a subject, which method at least comprises the above steps a) to c), and further comprises at least the step of administering the activated and loaded antigen-presenting cells (and in particular, but without limitation, dendritic cells) to said subject (i.e. as further described herein).

The invention also relates to a method for providing an antigen-presenting cell (and in particular, but without limitation, dendritic cell), or preparation or sample of APC's/DC's, for generating an immune response in a subject, which method at least comprises the steps of:
a) harvesting a sample or population of antigen-presenting cells from said subject (and in particular, of DC's and more in particular pDC's);
b) activating the antigen-presenting cells in said sample or population using the methods described herein; and
c) loading the antigen-presenting cells with one or more antigens against which the immune response is to be generated in said subject.

The invention further relates to a method for generating an immune response in a subject, which method at least comprises the above steps a) to c), and further comprises at least the step of administering the activated and loaded antigen-presenting cells (and in particular, but without limitation, dendritic cells) to said subject (i.e. as further described herein).

In the above methods, the subject may be a human subject (i.e. for immunotherapy or prophylaxis in human patients), but may also be another mammal, such as a rat, rabbit, dog, cat, cow, sheep, pig, horse or primate (either for veterinary purposes or a mammal that is used in or as an animal model).

Also, in both methods described above, the amount of APC's/DC's that is administered and the regimen according to which the APC's/DC's are administered are most preferably such that an immune response is generated in said subject (and in particular, a specific immune response against the antigen loaded onto the dendritic cells and/or against cells that carry or express said antigen). Reference is for example made to the dosing regimen mentioned herein.

Generally, the skilled person will be able to choose a specific antigen (or combination of antigens) for a specific disease or disorder to be prevented to treated. Generally, when the immune response is to be raised against a cell that is present in the subject to be treated (for example, a tumor cell), the antigen is most preferably an antigen that is expressed by said cell (for example, and without limitation, on the surface of said cell). Also, when the immune response is to be raised against a micro-organism that has infected the subject to be treated (such as a virus, bacterium or fungus), the antigen is most preferably an antigen that is expressed by said micro-organism.

Alternatively, it is also possible to use a suitable composition or preparation that is derived from the cell, tissue, or micro-organism against which the immune response is to be raised, such as a cell lysate, cell fraction, cell fragment or cell extract, suitable examples of which will be clear to the skilled person based on the disclosure herein. Such compositions or preparations may also be obtained or derived from cells, cell lines, tissues or micro-organisms that carry or express the same or similar antigens or antigenic determinants as the cell, tissue or micro-organism against which the immune response is to be raised, such that APC's/DC's that have been loaded using such a composition or preparation can be used to generate an immune response against the cell, tissue, or micro-organism against which the immune response is to be raised.

For example, when an immune response is to be raised against a tumor or tumor cell, the antigen may be protein or peptide that is expressed by the tumor cell, but may also be a suitable cell lysate, cell fraction, cell fragment or cell extract that has been obtained from a suitable cancer cell or suitable cancer tissue. This may for example be a cell lysate, cell fraction, cell fragment or cell extract that has been obtained from the tumor to be treated (i.e. obtained from tumor cells that have been removed from the patient to be treated), but may for example also be a cell lysate, cell fraction, cell fragment or cell extract that has been obtained (e.g. previously) from a similar tumor (e.g. from another patient), or a cell lysate, cell fraction, cell fragment or cell extract that has been obtained from a suitable tumor cell line. Similarly, when an immune response is to be raised against a micro-organism (e.g. a pathogenic micro-organism, such as those causing infectious diseases), the antigen may be protein or peptide that is expressed by the micro-organism, but may also be a suitable cell lysate, cell fraction, cell fragment or cell extract that has been obtained from the micro-organism or from the same or a similar strain of micro-organism.

As will be clear from the disclosure herein, and without limitation, activated and loaded APC's/DC's that have been obtained using the methods described herein may be used for immunotherapy of cancer in a subject, by loading the APC's/DC's with one or more antigens that are expressed by the cells of the tumor to be treated (also referred to in the art as “tumor-associated antigens” or “TAA's”, see for example the review by Tuyaerts et al. cited herein). Such antigens will be clear to the skilled person, and for example be an antigen that is present on the surface of or inside the cells of the tumor to be treated and/or that has been derived from the cells of the tumor to be treated. Reference is for example made to Van Der Bruggen et al., Immunological Reviews 2002, vol. 188, 51-64 and to the review by Novellino et al., Cancer Immunol. Immunother. (2005) 54: 187-207, which provide a lists of human tumor antigens that can be recognized by T-cells, which can also be used as antigens in the methods and compositions described herein.

Also, as mentioned herein, instead of such an antigen (which will often be a protein or polypeptide), it is also possible to use suitable (synthetic or semi-synthetic) tumour-specific peptide antigens, as well as a suitable cell lysate, cell fraction, cell fragment or cell extract that has been obtained from the cells of the tumor to be treated, or from a similar tumor or suitable tumor cell line.

Thus, in a specific, but non-limiting aspect, the invention relates to a composition for immunotherapy of cancer in a subject, which composition comprises an antigen-presenting cell (and in particular, but without limitation, dendritic cell) that has been activated and loaded using the methods described herein, wherein said antigen-presenting cell has been loaded with one or more antigens that are expressed by, are present on the surface of, and/or have been derived from the cells of the tumor to be treated.

The invention also relates to a method for providing such a composition, which comprises the above steps a) to c), in which the antigen-presenting cell is loaded with one or more antigens that are expressed by, are present on the surface of, and/or have been derived from the cells of the tumor to be treated.

The invention also relates to a method for cancer immunotherapy in a subject in need of such immunotherapy, which method at least comprises the step of administering to said subject a preparation or sample of activated and loaded antigen-presenting cells (and in particular, but without limitation, dendritic cells) as described herein, wherein said antigen-presenting cells have been loaded with one or more antigens that are expressed by, are present on the surface of, and/or have been derived from the cells of the tumor to be treated. In such a method, the preparation or sample of activated and loaded dendritic cells is preferably obtained by a method which comprises the above steps a) to c), in which the antigen-presenting cells are loaded with one or more antigens that are expressed by, are present on the surface of, and/or have been derived from the cells of the tumor to be treated.

In another specific, but non-limiting aspect, the invention relates to a composition for generating an immune response against one or more tumor cells, which composition comprises an antigen-presenting cell (and in particular, but without limitation, dendritic cell) that has been activated and loaded using the methods described herein, wherein said antigen-presenting cell has been loaded with one or more antigens that are expressed by, are present on the surface of, and/or have been derived from said tumor cell(s). Again, the invention also relates to a method for providing such a composition, which comprises the above steps a) to c), in which the antigen-presenting cells are loaded with one or more antigens that are expressed by, are present on the surface of, and/or have been derived from the cells of the tumor to be treated.

The invention also relates to a method for generating, in a subject, an immune response against one or more tumor cells present in said subject, which method at least comprises the step of administering to said subject a preparation or sample of activated and loaded antigen-presenting cells (and in particular, but without limitation, dendritic cells) as described herein, wherein said antigen-presenting cells have been loaded with one or more antigens that are expressed by, are present on the surface of, and/or have been derived from said tumor cell(s). Again, in such a method, the preparation or sample of activated and loaded antigen-presenting cells is preferably obtained by a method which comprises the above steps a) to c), in which the antigen-presenting cells are loaded with one or more antigens that are expressed by, are present on the surface of, and/or have been derived from the cells of the tumor to be treated.

The invention also relates to an antigen-presenting cell (and in particular, but without limitation, dendritic cell) that has been activated using a vaccine (and/or one or more antigenic components as described herein) and loaded with one or more antigens that are expressed by and/or derived from a tumor, for use in immunotherapy of cancer. The invention further relates to a composition comprising such an antigen-presenting cell.

The compositions and methods described herein may for example be used in the prevention and treatment of the following tumors: melanoma, colon carcinoma, renal cell carcinoma, mesothelioma, breast cancer, prostate cancer, glioblastoma, myeloma, lymphoma, bladder cancer, head and neck cell carcinoma, sarcoma's, pediatric solid tumors, etc. The compositions and methods described herein may also be used to treat metastases and/or to prevent metastases from spreading in a subject to be treated. Again, the clinician will be able to determine a suitable treatment regimen for the treatment of such tumors in the subject to be treated, using the activated and loaded dendritic cells described herein. Also, in such a treatment regimen, the use of the activated and loaded dendritic cells may be suitably combined with conventional treatments of cancer, such as radiation treatment, surgery and treatment with cytostatic drugs known per se.

Also, as mentioned above, the methods described herein can be used to activate and/or load one or more of these APC's either systemically or in the organ(s) or tissue(s) in which the tumour is present (e.g. by administration to said tissue or organ, and/or by administration into the tumor or into the immediate surroundings of the tumor).

The methods described herein can for example be used to activate and/or load one or more specific APC's in the tissue or organ in which they (and the tumor to be treated) occur. For example, methods described herein can be used to activate and/or load astrocytes/microglial cells in the brain, Ito cells/Kupfer cells and/or liver sinusoidal endothelial cells (LSEC) in the liver, alveolar macrophages in the lungs, osteoclasts in bone, or sinusoidal lining cells in the spleen.

It will be clear to the skilled person that the above method may generally comprise ex vivo activation and loading of the APC's/DC's (or suitable precursors for the DC's), which may then be suitably administered to the subject to be treated (and in particular, returned to the subject from which they were originally harvested. Alternatively, as mentioned herein, it is also possible to use a sample or population of APC's/DC's that have been obtained from another subject, and/or to use a sample or population of DC's (or APC's, where applicable) that has been cultivated in vitro, for example from suitable precursors as mentioned herein).

It is also envisaged that the methods described herein may be used to activate APC's/DC's in vivo, and in particular to generate a cytotoxic immune response against one or more tumor cells in the subject to be treated.

For example, the methods and compositions described herein can be used to activate APC's/DC's and/or to generate an (antigen-specific) immune response in the body of a subject to be treated (i.e. in situ), for example for tumour immunotherapy, for any other use of immunotherapy as described herein, and/or for immunomodulation and/or to induce tolerance in a subject against one or more specific antigens (as further described herein). Generally, this may be performed by suitably administering a vaccine or antigenic compound as described herein (in)to the body of the subject (i.e. into the circulation of the patient or to a part, tissue or organ of the body), and optionally also administering the desired antigen or antigens (in)to the body of the patient (i.e. into the circulation of the patient or to a part, tissue or organ of the body), either as essentially simultaneous administrations or according to a suitable administration regimen, such that at least one antigen-presenting cell (and in particular, but without limitation, dendritic cell) in the body of the subject is activated (as described herein) and optionally also loaded (as described herein) with the desired antigen(s). For example, but without limitation, the vaccine or antigenic component(s), and optionally the antigen(s), may be administered directly into the part(s) or tissues of the body where the immune response is to be raised. For instance, for the immunotherapy of tumors using the methods described herein, the vaccine or antigenic component(s), and optionally one or more antigen(s) that are specific for the tumor to be treated (as described herein), may be administered directly into the tumor and/or into the tissue that immediately surrounds the tumor.

Thus, in another aspect, the invention relates to a vaccine, to an antigenic component or to a pharmaceutical composition comprising at least one antigenic component for (use in) activating antigen-presenting cells (and in particular, but without limitation, dendritic cells) by administration to the body of a subject to be treated (i.e. into a part, tissue or organ of a subject to be treated, such as a tumor).

The invention also relates to an antigen (as described herein) or pharmaceutical composition comprising at least one antigen for raising an immune response in a subject, by means of administering said antigen or composition to the body of a subject to be treated (i.e. into a part, tissue or organ of a subject to be treated, such as a tumor), together with a vaccine, an antigenic component or a pharmaceutical composition comprising at least one antigenic component for activating antigen-presenting cells (and in particular, but without limitation, dendritic cells) (i.e. by essentially simultaneous administration or according to a suitable administration regimen). The antigen(s) or pharmaceutical composition comprising the antigen(s) may also be provided as a kit of parts together with the vaccine, the antigenic component(s) or a pharmaceutical composition comprising the antigenic component(s), which kit of parts may be as further described herein.

In one specific aspect, the antigen or antigen(s) may be tumor-derived, tumor-specific and/or tumor-associated antigens (i.e. as further described herein, including suitable tumor cell lysates or fractions); and the vaccine, antigenic component(s) or pharmaceutical composition comprising the antigenic components, as well as the antigen(s) or pharmaceutical composition comprising the antigen(s) may be suitable or intended for administration into a tumor or into the tissues that surround a tumor.

In another aspect, the invention also provides compounds, constructs or complexes that can be used to activate antigen-presenting cells, that can be used in the methods described herein, and/or that can be administered to a subject (e.g. systemically or in or near the site where the immune response is to be raised, such as in or in the immediate vicinity of a tumour to be treated) in order to activate at least one antigen-presenting cell (such as a dendritic cell) in the body of said subject, and optionally also to raise an immune response in said subject against one or more desired antigens.

As further described herein, such a compound, construct or complex may generally comprise:
(i) a first moiety that is capable of targeting the compound, construct or complex towards the antigen-presenting cell(s) to be activated (either in vitro, ex vivo or in vivo, i.e. in the body of a subject to be treated). This first moiety may for example be an antibody or antibody fragment directed against the antigen-presenting cell, as further described herein;
   and in addition one or both of:
(ii) an antigenic compound (i.e. for activating the antigen-presenting cell(s), as further described herein);
   and/or
(iii) the desired predetermined antigen or antigens (as defined herein) against which the immune response is to be raised. For example, when an immune response is to be raised against a tumor cell, this may be any suitable material or antigen that is derived from said tumor cell (or from an equivalent or similar tumor cell or cell tumor line), such as cellular antigens (as described herein), proteins, polypeptides, or RNA.

As further described herein, such a compound, complex or construct may be targeted towards (e.g. directed against) any suitable or desired “antigen-presenting cells” (as described herein), and may in particular be targeted towards dendritic cells.

Thus, in another aspect, the invention relates to a compound, construct or complex for activating at least one dendritic cell, comprising: (i) a first moiety that is capable of targeting the compound, construct or complex towards an APC (and in particular, but without limitation, to a DC); and (ii) an antigenic compound; and optionally (iii) one or more desired antigens. The invention further relates to a compound, construct or complex for raising an immune response in a subject against one or more desired antigens, comprising: (i) a first moiety that is capable of targeting the compound, construct or complex towards an APC (and in particular, but without limitation, to a DC); and optionally (ii) an antigenic compound; and (iii) the one or more desired antigens.

The first moiety may for example be a binding unit or binding domain that is capable of specifically binding to an APC (and in particular, but without limitation, to a DC) and/or to an antigen or antigenic component expressed by an APC (and in particular, but without limitation, a DC). Some non-limiting examples of binding units that are suitable for this purpose are immunoglobulins or immunoglobulin fragments, such as an antibody, antibody fragment or antibody-derived construct (for example, a Fab fragment, ScFv, V_{H} domain, V_{L} domain or single domain antibody).

The antigenic compound(s) may be any suitable antigenic compound(s) as described herein, and may thus for example be, again without limitation, a bacterium, virus, viral particle, nucleic acid that is derived from a bacterium or virus, or any other suitable composition or preparation that can be (or has been) derived from a bacterium or virus (such as a bacterial or viral lysate, fragment, fraction, supernatant or suspension); or any other suitable antigenic component that is used in a vaccine. Similarly, the antigen may be any suitable antigen(s) as described herein.

In such a compound, complex or construct, the first moiety and the antigenic compound(s), and optionally the antigen(s), may be suitably linked to each other or associated with each other. For example, the first moiety, the antigenic component(s), and optionally the antigen(s), may be covalently linked to each other, either directly or via a suitable linker or spacer, such as a peptidic linker (for this purpose any suitable linkers or spacer known per se can be used, and such linkers and spacers will be clear to the skilled person based on the disclosure herein). Alternatively, in such a complex or construct, the first moiety directed against the DC may be linked to a second moiety that can bind one or more antigenic components (to which the antigenic component(s) may be bound), and optionally to a third moiety for binding the antigen(s) (to which the antigens may be bound). It is also possible to provide a construct that comprises the first moiety linked to an antigenic component and that further comprises a moiety for binding the antigen(s) to which the antigens may be bound (i.e. a “third moiety” as referred to in the previous paragraph).

Another construct that is suitable for use in the methods described herein may comprise a first binding unit directed against an APC (and in particular, but without limitation, a DC) and either a desired antigen or a moiety for binding an antigen (i.e. a “third moiety” as referred to in the preceding paragraphs) to which antigens may be bound. Such a construct may be used to direct the desired antigen(s) to an APC (and in particular, but without limitation, to a DC) that has been activated in vivo or in situ with a vaccine or antigenic component using the methods described herein (i.e. by administering the vaccine, the antigenic component or a composition comprising the same to the body of a subject or to a specific part, tissue or organ of a subject).

In the above complexes or constructs, the second and third moieties (if present) may again be any suitable binding unit or binding domain, such as an antibody, antibody fragment or antibody-derived construct (for example, a Fab fragment, ScFv, V_{H} domain, V_{L} domain or single domain antibody). Also, in such a construct, the first moiety for binding the APC's/DC's, the second moiety for binding the antigenic component (or alternatively the antigenic component itself), and optionally the third moiety for binding the antigen(s) (or alternatively the antigen itself), may again be suitably linked to each other, i.e. directly or via a suitable linker or spacer, such as a peptidic linker. Such constructs, as well as complexes that comprise such constructs, the antigenic components (if these do not form part of the construct) and optionally the desired antigen(s), form further aspects of the invention.

The invention also relates to a pharmaceutical composition that comprises such a compound, complex or construct. Furthermore, if the compound, complex or construct does not comprise the antigenic compound and/or the antigen(s), respectively, these may also be included in this pharmaceutical composition (or alternatively, these may be administered and/or used as part of a separate pharmaceutical composition). Also, all the pharmaceutical compositions described herein may contain one or more pharmaceutically acceptable carriers, and may for example be in a form suitable for injection, such as a suspension or solution in a physiological buffer or solution.

Again, such compounds, complexes, constructs or compositions may be administered to a subject to be treated, optionally together with one or more antigenic compounds (where such antigenic compounds do not form part of the compound, complex or construct) and/or the one or more antigens (where such antigens do not form part of the compound, complex or construct), i.e. in such a way that at least one antigen-presenting cell (and in particular, but without limitation, dendritic cell) in the body of the subject is activated (as described herein) and optionally also loaded with the desired antigen(s). This may again be performed by essentially simultaneous administration or by administration according to a suitable administration regimen, to the body of a patient or to a specific part, organ or tissue of the body of a subject.

Also, for this purpose, the compounds, complexes or constructs (or a pharmaceutical composition comprising the same) may be provided as a kit of parts, together with one or more antigenic compounds or a pharmaceutical composition comprising the same (i.e. where the compound, complex or construct itself does not comprise an antigenic compound), and/or together with one or more antigens or a pharmaceutical composition comprising the same (i.e. where the compound, complex or construct itself does not comprise an antigen). Again, such a kit of parts may be as further described herein.

Based on the disclosure herein, it will also be clear to the skilled person that the compounds, complexes or constructs (or a pharmaceutical composition comprising the same, or the above kits), may also be used in methods for activating and/or loading APC's (and in particular DC's) in vitro and/or ex vivo, e.g. using the methods described herein. It will furthermore be clear to the skilled person that it may also be possible to use a suitable combination of ex vivo steps and in vivo (e.g. in situ or systemic) steps, as long as by doing so, the intended or desired antigen-presenting cells are activated and/or loaded and/or the intended or desired immune response is raised, at least at the site or in the tissue or organ where the antigen-presenting cells are to be activated and/or where the immune response is to be raised.

Again, as in the further description herein, said immune response may be any suitable immune response (such as a T-cell or B-cell mediated immune response) and is most preferably a specific immune response against the one or more (predetermined) antigens.

Again, according to a specific aspect, the antigen may be a tumor-derived, tumor-specific or tumor-associated antigen, in which case the complex or construct may be administered (optionally together with the vaccine, antigenic component or antigen(s), if these do not form part of the compound, complex or construct), into the tumor to be treated.

The methods, compositions and kits for activating and optionally loading APC's (and in particular DC's) in vivo or in situ as described herein may for example be used after surgery (or in the course of a surgical procedure) in order to generate an immune response against the tumor that is removed, to treat metastases and/or to prevent metastases from spreading, and generally to boost the immune system following such surgery.

Activated and loaded APC's (and in particular DC's) that have been obtained using the methods described herein may also be used for immunotherapy (curative and/or as prophylaxis, i.e. as a vaccine; and/or for alleviating the inflammatory responses or other symptoms of infection through tolerization) of infectious diseases in a subject, by loading the APC's/DC's with one or more antigens that are expressed by the micro-organism that has infected the subject to be treated (or to which the subject to be treated may be exposed). Such antigens may depend on the specific micro-organism (which may for example be a bacterium, virus or fungus), and may be suitably chosen by the skilled person based on the disclosure herein. For a non-limiting example of the use of dendritic cell vaccination in the treatment of infectious diseases, reference is for example made to Perruccio et al., Blood Cells, Molecules and Diseases 33 (2004), 248-255.

The invention also relates to compositions for the prevention and/or treatment of infectious diseases in a subject, to methods for preparing such compositions, and to methods for the prevention and/or treatment of infectious diseases in a subject, which compositions and methods may essentially be as described herein for the compositions and methods for the immunotherapy of cancer, but using one or more antigens that are expressed by the relevant pathogenic and/or infectious micro-organism (instead of antigens that are expressed by the tumor cells).

The invention also relates to an antigen-presenting cell (and in particular, but without limitation, dendritic cell) that has been activated using a vaccine (and/or one or more antigenic components as described herein) and loaded with one or more antigens that are expressed by and/or derived from a pathogenic and/or infectious micro-organism, for use in immunotherapy of infectious diseases. The invention further relates to a composition comprising such an antigen-presenting cell.

As it is known that dendritic cells may not only be used for raising an immune response in a subject, but may also be used for immunomodulation and/or to induce tolerance in a subject (such as peripheral tolerance, see for example the review by Xiao et al., J. Immunother., Vol. 29, No. 5 (2006), 465-471), the activated and loaded DC's that have been obtained using the methods described herein may also be used to induce DC-mediated tolerance in a subject, for example for immunotherapy (curative and/or as prophylaxis), for example for the treatment of auto-immune diseases, of inflammatory diseases or disorders (such as rheumatoid arthritis or asthma), transplant rejections or allergies in a subject. According to this aspect, the methods of the invention may be used to generate so-called “tolerogenic” DC's for use in therapy (see again the review by Xiao et al.). As a non-limiting example thereof, reference is made to Kuipers and Lambrecht, Vaccine 23 (2005), 4577-4588, who describe the use of tolerogenic DC's in the prevention and treatment of asthma (in particular atopic asthma). It is envisaged that suitable APC's (which have also been suitably loaded) may be used in a similar or equivalent manner.

The invention therefore also relates to antigen-presenting cells (and in particular, but without limitation, dendritic cells) (and to compositions comprising the same) that can be used for immunomodulation in a subject. The invention further relates to antigen-presenting cells (and in particular, but without limitation, dendritic cells) (and to compositions comprising the same) that can be used for inducing tolerance in a subject against one or more antigens, which the antigen-presenting cells have been activated and loaded using the methods described herein, i.e. with the antigens against which tolerance is to be induced in said subject. Such antigen-presenting cells and compositions may for example be used for the prevention and/or treatment of auto-immune diseases, of inflammatory diseases or disorders (such as rheumatoid arthritis or asthma), of transplant rejections and/or of allergies in a subject, by loading the antigen-presenting cells with one or more antigens that are involved in the undesired or excessive immune response that is involved in the relevant auto-immune disease, inflammatory disease, transplant rejection or allergy.

Other applications and uses of the antigen-presenting cells (and in particular, but without limitation, dendritic cells) , compositions and methods described herein will be clear to the skilled person based on the disclosure herein. The invention further relates to a kit of parts that at least comprises one or more antigen-presenting cells (and in particular, but without limitation, dendritic cells) and a vaccine for activating the antigen-presenting cells (or alternatively, one or more antigenic components as defined herein, or a composition comprising one or more such antigenic components). In such a kit of parts, the antigen-presenting cells and the vaccine (or antigenic components) will usually be present in separate containers, which may be packaged together, optionally with instructions for use or other product information. Such a kit of parts may optionally also contain one or more antigens for loading the antigen-presenting cells (i.e. once they have been activated with the vaccine or the antigenic component), which will usually also be present in a separate container.

The invention also relates to a kit of parts that can be used to activate and load antigen-presenting cells (and in particular, but without limitation, dendritic cells) with one or more desired antigens, which kit of parts at least comprises a vaccine for activating the antigen-presenting cells (or alternatively, one or more antigenic components as defined herein or a composition comprising one or more such antigenic components) and the one or more desired antigens. In such a kit of parts, the vaccine (or antigenic components) and the antigens will usually be present in separate containers, which may be packaged together, optionally with instructions for use or other product information.

The invention further relates to a kit of parts that at least comprises one or more antigen-presenting cells (and in particular, but without limitation, dendritic cells) that have been activated using one of the methods described herein, as well as one or more desired antigens for loading the activated antigen-presenting cells. In such a kit of parts, the activated antigen-presenting cells and the antigens will usually be present in separate containers, which may be packaged together, optionally with instructions for use or other product information.

In the above kits, the antigen-presenting cells (and in particular, but without limitation, dendritic cells), vaccines, antigenic components and/or antigens may be as further described herein.

Finally, although the invention has been described in detail with reference to activating (and loading) antigen-presenting cells (such as DC's), according to another specific aspect of the invention, it is envisaged that the methods, vaccines, antigenic components, compounds, constructs, complexes and kits described herein may also be used to activate other cells that carry one or more of the TLR's mentioned herein. These may for example, but without limitation, be cells that are involved in the immune system. Some non-limiting examples of cells that may be activated using the methods, vaccines, antigenic components, compounds, constructs, complexes and kits described herein are T-cells, B-cells, natural killer cells (NK-cells), natural killer T-cells (NKT-cells), regulatory T cells, cytotoxic T-lymphocytes (CTL's), etc.

It is envisaged that this aspect of the invention may for example be used for modulating (e.g. increasing or reducing) one or more immune responses in a subject.

Certain TLRs are expressed on T lymphocytes and can be modulated by TLR ligands. For example, TLR2, TLR3, TLR5 and TLR9 act as co-stimulatory receptors to enhance proliferation and effector function (i.e. cytokine production) after T cell receptor stimulation of T cells. Furthermore, modulation of the suppressive activity of naturally occurring regulatory T cells is observed after TLR2, TLR5 or TLR8 triggering. The direct responsiveness of T cells to TLR ligands offers new perspectives for the immunotherapeutic manipulation of T cell responses in for example infectious diseases, cancer and autoimmunity (ref Current Opinion in Immunology 2007, Kabelitz).

Some preferred, but non-limiting aspects of the invention are:
1. An in vitro or ex vivo method for providing a composition that comprises at least one activated dendritic cell, which method at least comprises the steps of: a) providing a composition that comprises at least one dendritic cell, in which said composition comprises at least one plasmacytoid-derived dendritic cell and/or at least one myeloid-derived dendritic cell; b) activating said dendritic cell by contacting it with a vaccine.
2. Method according to claim 1, in which the at least one dendritic cell is brought into a state in which it is capable of stimulating T-cells and/or a T-cell mediated response.
3. Method according to any of the preceding claim 1, in which the vaccine comprises a formulation or preparation of one or more antigenic components that are capable of activating one or more plasmacytoid-derived dendritic cells and/or one or more myeloid-derived dendritic cells through the interaction with one or more dsRNA sensors and/or toll-like receptors (TLR's) that are expressed by the dendritic cells to be activated.
4. Method according to claim 3, in which the dendritic cells are plasmacytoid-derived dendritic cells, and in which the one or more antigenic components are capable of activating plasmacytoid-derived dendritic cells by interaction with one or more of the following TLR's expressed by the plasmacytoid-derived dendritic cells: TLR-7, TLR-8, and/or TLR-9.
5. Method according to claim 3, in which the vaccine comprises one or more of the following antigenic components: inactivated, weakened or attenuated bacteria or viruses; inactivated, weakened or attenuated viral particles; DNA, single stranded RNA or double stranded RNA that is contained in or encoded by bacteria or viruses; or any other suitable antigenic components that are based on, and/or that have been derived from, micro-organisms, such as bacterial or viral proteins, as well as cell fragments or cell fractions that have been derived from bacteria, viruses or other suitable microorganisms.
6. A plasmacytoid-derived dendritic cell and/or myeloid-derived dendritic cell that has been activated using a method as defined in any of claim 1.
7. Method according to any one of claim 1; wherein in a further step c) said dendritic cell is loaded with the one or more desired antigens.
8. Method according to claim 7, in which, in step c), the one or more desired antigens is one or more tumour-associated antigens.
9. Method according to claim 7, for providing one or more tolerogenic dendritic cells.
10. Method according to claim 9, for providing one or more tolerogenic dendritic cells forthe prevention and/or treatment of an auto-immune disease, of an inflammatory disease or disorder such as rheumatoid arthritis or asthma, of a transplant rejection and/or of an allergy in a subject.
11. Method for providing at least one plasmacytoid-derived dendritic cell and/or at least one tolerogenic myeloid-derived dendritic cell for use in immunotherapy in a subject, which method at least comprises the steps of: a) harvesting a sample or population of said cells from said subject; b) activating these cells in said sample or population using a vaccine; and c) loading these cells with one or more antigens that are suitable for immunotherapy in said subject.
12. Method according to claim 11, wherein said activated dendritic cells is loaded with one or more tumor-associated antigens.
13. Method for immunotherapy in a subject, which method at least comprises the steps of: a) harvesting a sample or population of plasmacytoid-derived dendritic cell and/or myeloid-derived dendritic cell from said subject; b) activating these cells in said sample or population using a vaccine; c} loading these cells with one or more antigens that are suitable for immunotherapy in said subject; and d) administering the activated and loaded dendritic cells to said subject.
14. Method according to claim 13, for immunotherapy of cancer in a subject, in which, in step c), the activated dendritic cell is loaded with one or more tumor-associated antigens; and/or with one or more suitable (synthetic or semi-synthetic) tumour-specific peptide antigens; and/or with a celilysate, cell fraction, cell fragment or cell extract that has been obtained from a tumor cell or tumor cell line.
15. A kit for providing activated dendritic cells as defined in claim 1 that have been loaded with one or more desired antigens, at least comprising a vaccine for activating the cells and the one or more desired antigens.

The invention will now be illustrated by means of the following non-limiting Experimental Part and Figures, in which:

FIG. 1 shows phenotype and IFN-alpha production by pDCs. Surface marker expression was assessed by flow cytometry and type I IFN production was measured by ELISA. FIG. 1A: Expression levels of the surface molecules CD80, CD83, CD86, MHC class I and MHC class II on pDCs after 18 hours of cultivation with IL-3 and 18 hours of activation with either CpG C or FSME vaccine. FIG. 1B: IFN-alpha production was measured in the supernatants of pDCs after 18 hours of cultivation/activation with IL-3, CpG C or FSME vaccine. Means±SD represent IFN-alpha production of three different donors. (*p<0.05);

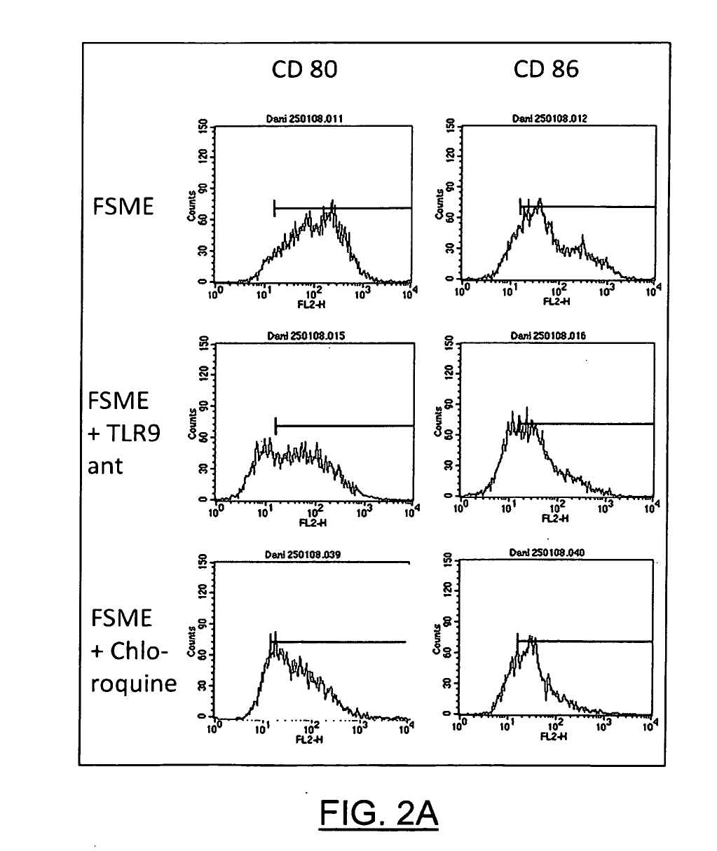

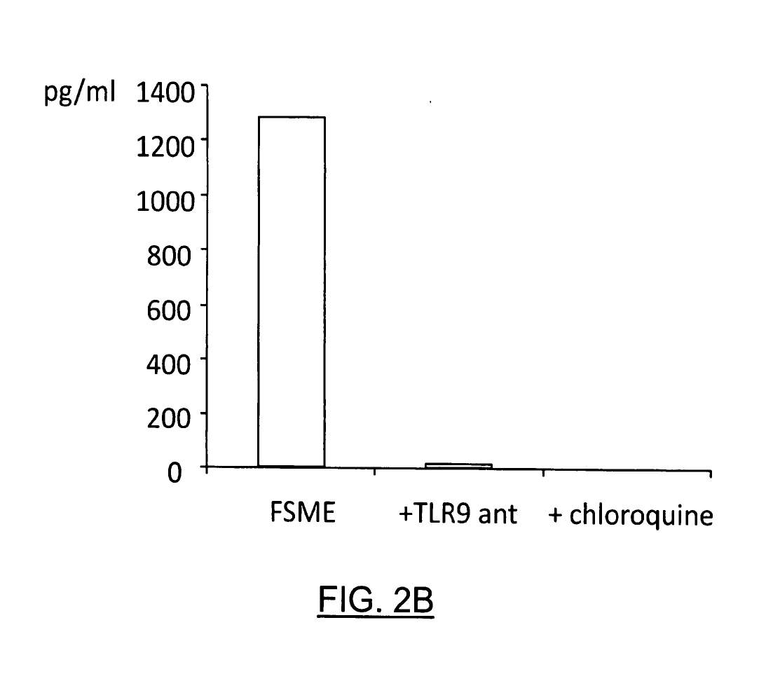

FIG. 2 shows the activation of pDCs with FSME vaccine is mediated via TLR-9 signaling. FIG. 2A: Expression of the co-stimulatory molecules CD80 and CD86 after activation FSME vaccine in the presence or absence of a TLR-9 antagonist or chloroquine. FIG. 2B: IFN-alpha production was measured in the supernatants of pDCs after 18 hours of activation with FSME vaccine in the presence or absence of a TLR-9 antagonist or cloroquine.

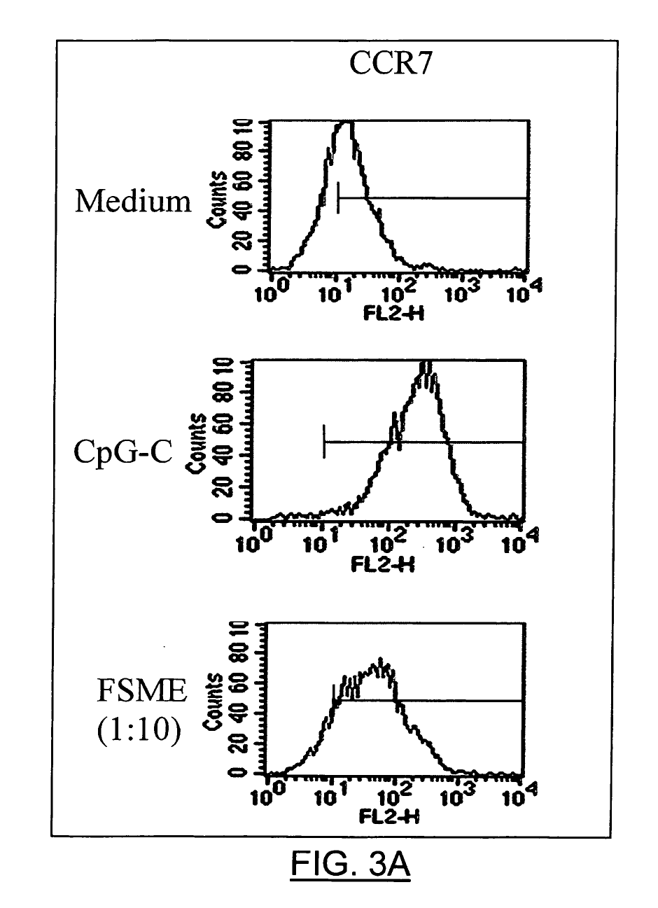

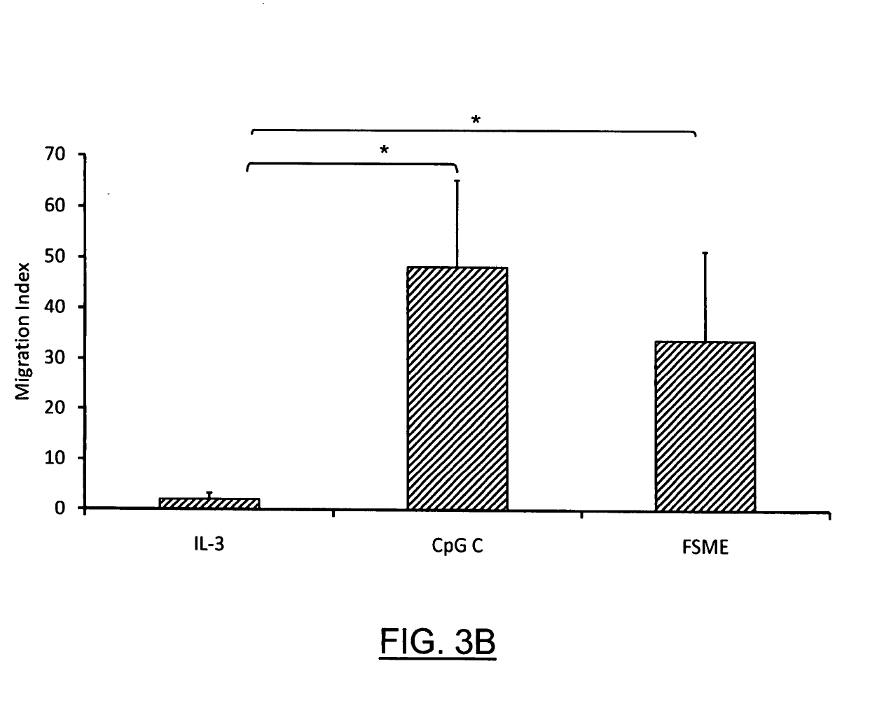

FIG. 3 shows the migratory capacity of pDCs after activation. FIG. 3A: Surface expression of CCR7 is up regulated on pDCs after overnight incubation with CpG C and FSME vaccine compared to IL-3 cultivation. FIG. 3B: 1*10⁵ overnight stimulated pDCs were allowed to migrate towards 100 ng/ml CCL21 for two hours. Spontaneous migration was assessed through migration of pDCs in the absence of CCL21. (*p<0.05)

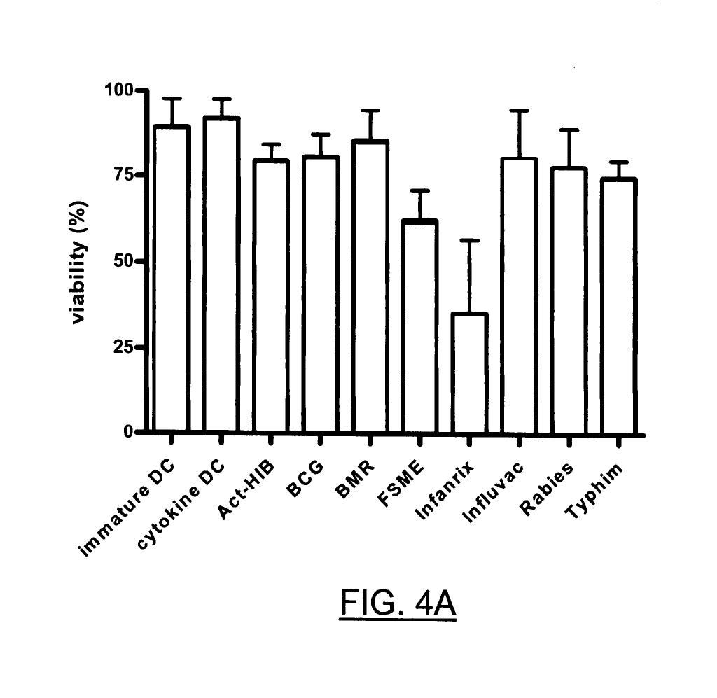

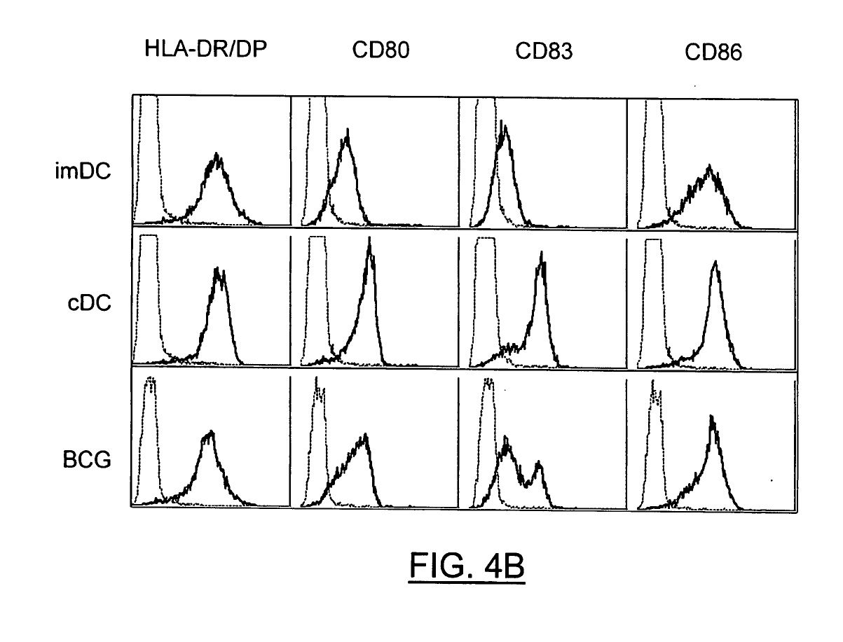

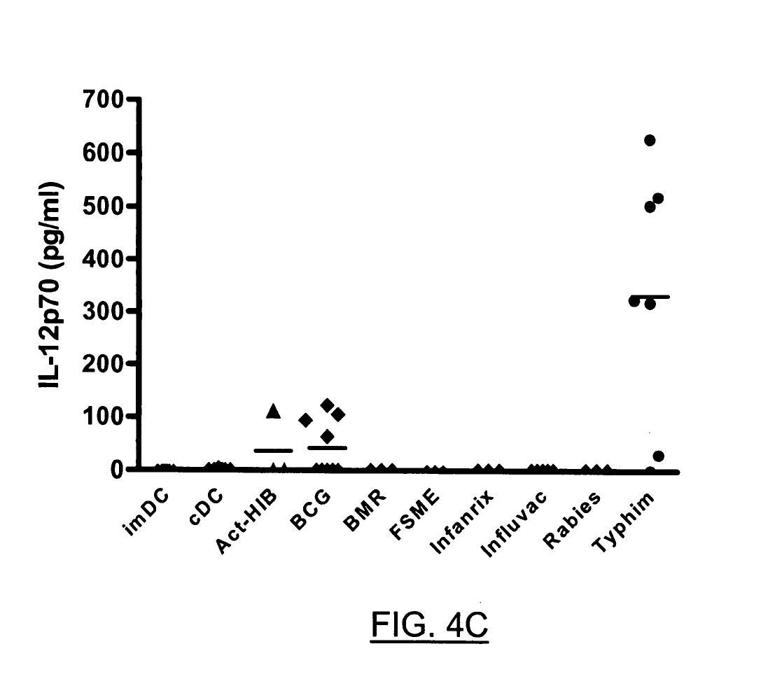

FIG. 4 shows that vaccines induce DC maturation. Immature DC were incubated with the conventional cytokine cocktail (TNF-alpha, IL-6, IL-1 beta, and PGE₂) or with different preventive vaccines for 48 hr. A. Viability was analysed by Trypan blue exclusion. Data are presented as the mean±SD of three independent experiments performed with DC from different donors. B. The expression of maturation markers HLA-DR/DP, CD80, CD83, CD86 (bold line) was measured by flow cytometry. The thin line represents the isotype control. C. 48 hr after addition of the vaccines IL-12p70 secretion was measured in the supernatant by ELISA. Per condition each symbol represents one donor. Means are shown for each vaccine.

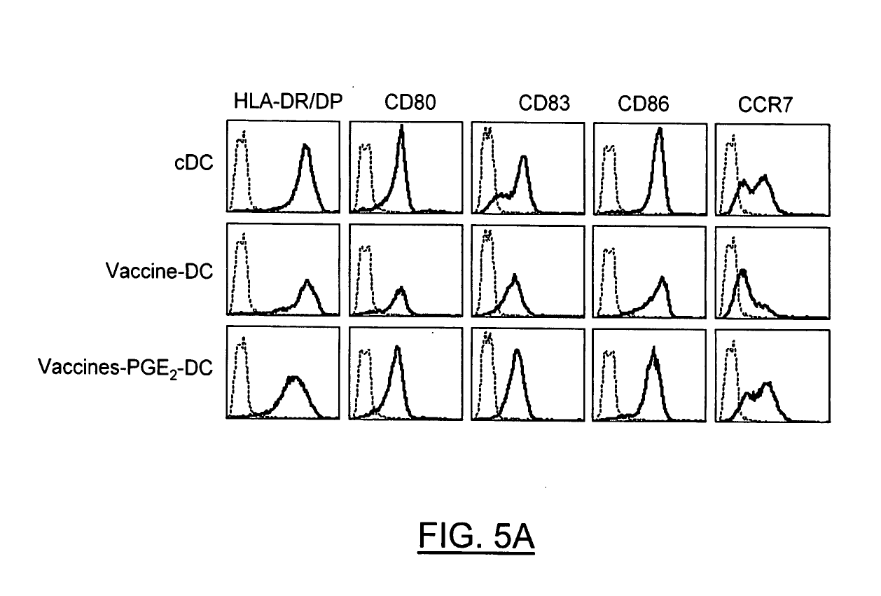

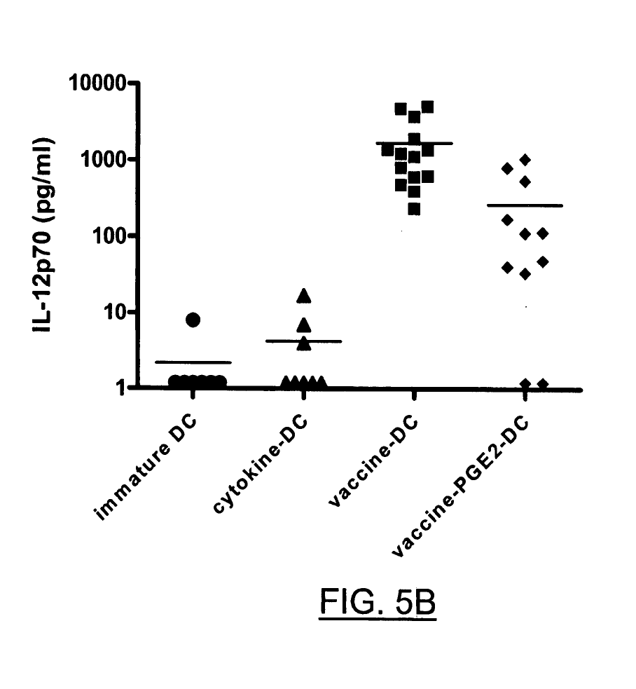

FIG. 5 shows that combining vaccines have synergistic effect on DC maturation. DC were matured for 48 hr with the conventional cytokine cocktail (TNFα, IL-6, IL1β, and PGE₂), preventive vaccines (BCG, Typhim, Influvac/Act-HIB), or vaccines with or without PGE₂ and the expression of maturation markers and IL-12p70 production was evaluated. A. The expression of maturation markers HLA-DR/DP, CD80, CD83, CD86, and CCR7 (bold line) was measured by flow cytometry. The thin line represents the isotype control. B. IL-12p70 production was measured by ELISA in the supernatant of DC cultures 48 hr after maturation. Per condition each symbol represents one donor. Means are shown for each maturation cocktail.

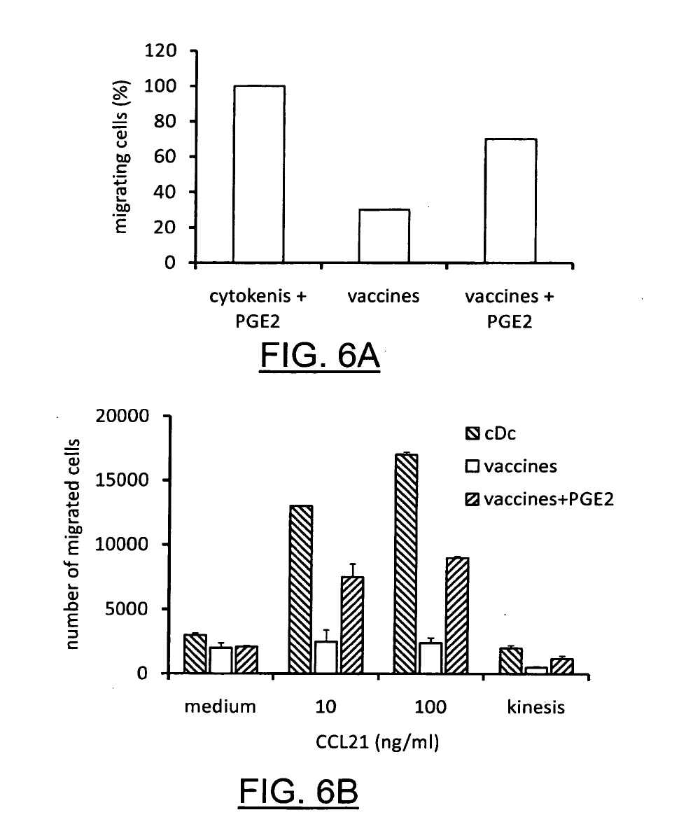

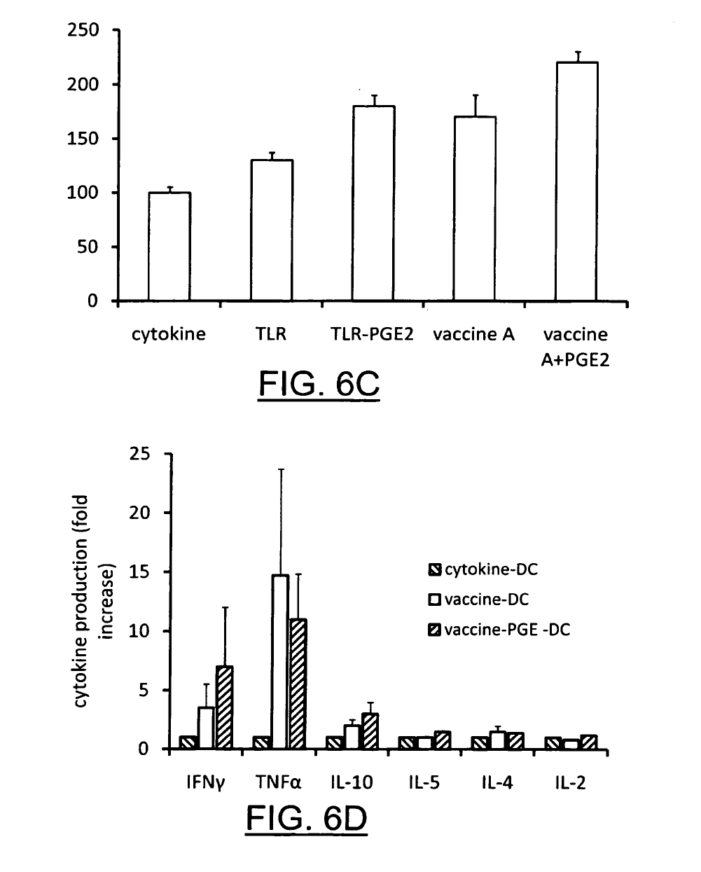

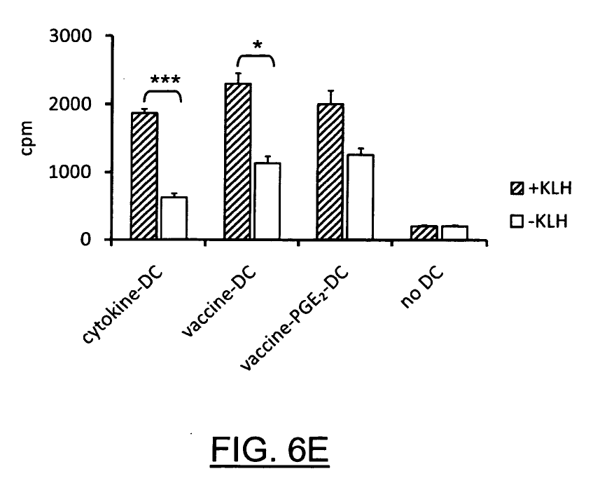

FIG. 6 shows that vaccine-DC are suitable for vaccination of melanoma patients. A. Random migration on fibronectin. Cytokine-DC, vaccine-DC, and vaccine-PGE2-DC were added to a fibronectin-coated plate and migration of individual cells was monitored for 60 min. Data represent the percentage of migrating cells of 50 cells pooled fromone experiment. B. CCR7-mediated chemotaxis of cytokine-DC, vaccine-DC, and vaccine-PGE2-DC was determined by the number of cells that had migrated into the lower compartment of a transwell system containing increasing concentrations of CCL21, counted by flow cytometry. To measure spontaneous migration, cells were incubated in a transwell without CCL21 in the upper and lower compartment (medium) or with CCL21 in both compartments (kinesis). The graph shows means of duplicates (±SD) and is from one representative experiment out of three performed (from different donors). C+D. The allostimulatory capacity of the DC was tested in a mixed lymphocyte reaction (MLR). Allogeneic PBL were cocultured with cDC, vaccine-DC and vaccine-PGE₂-DC and T cell proliferation was measured by incorporation of tritiated thymidine (C). The profile of cytolines secreted by PBL upon contact with cDC vaccine-DC and vaccine-PGE₂-DC was measured by cytokine bead array (D). The graph shows the fold change in the cytokine production of vaccine-DC and vaccine-PGE₂-DC relative to cDC of two different donors. The table presents the mean±SEM concentration (pg/ml) of each cytokine absolute numbers for all conditions. E. KLH-specific proliferation of PBL from a patient vaccinated with KLH-loaded DC. PBL were cocultured with autologous DC matured with the cytokine cocktail, vaccines or vaccines with PGE₂ with or without KLH. Proliferation was measured by incorporation of tritiated thymidine. Black bars represent DC loaded with KLH. Gray bars represent DC without KLH. The figure shows mean±SD of one representative expemeriment out of three performed.

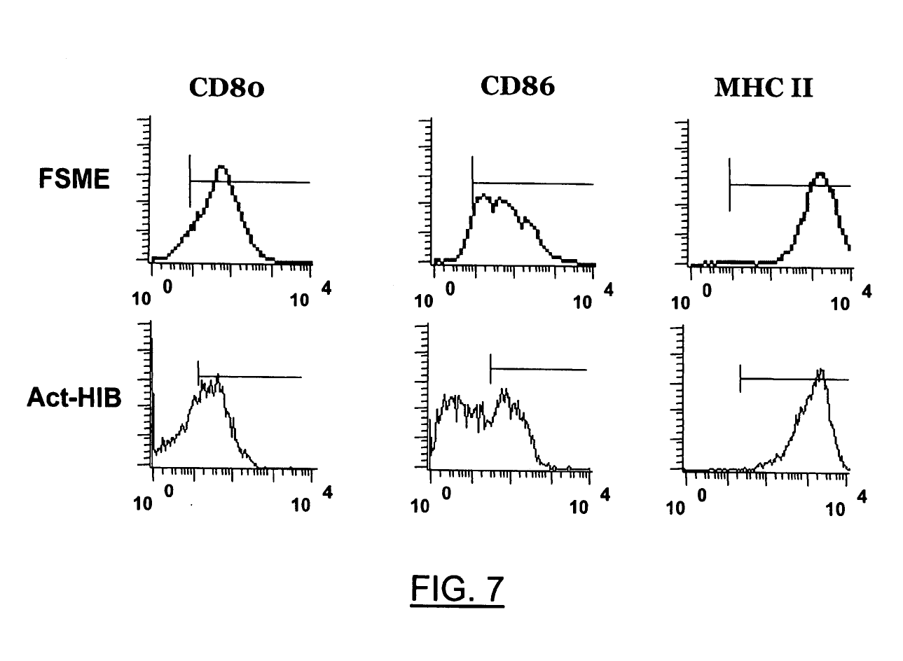

FIG. 7 shows the phenotype of pDC's (expression of CD80 and CD86 and MHC class II) after activation with FSME (upper panel) or Act-Hib (lower panel)

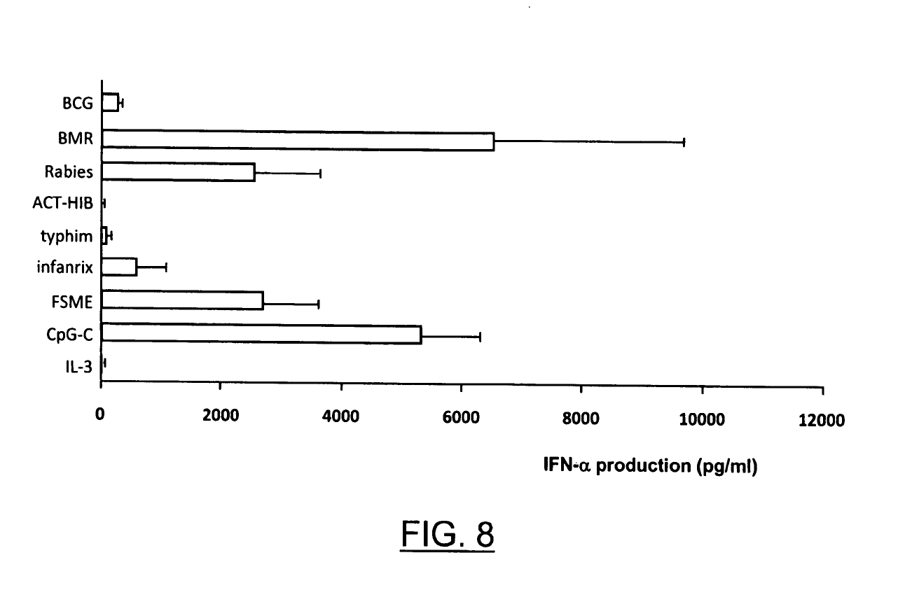

FIG. 8 shows the production of IFN-alpha by pDC's after activation with different vaccines.

### EXPERIMENT PART

Unless indicated or defined otherwise, all terms used herein have their usual meaning in the art, which will be clear to the skilled person. Reference is for example made to the handbooks mentioned herein, as well as standard handbooks in the fields of molecular biology and immunology, such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005).

Also, unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein.

Dendritic cells are one of the antigen presenting cells of the body that are able to recognize proteins, take them up and can initiate a de novo immune response against such proteins. In current practice, DC's that have been loaded with tumour antigens are used in the treatment of cancer. There are different types of antigen presenting cells including DC's, which occur in an immature or undifferentiated state and in a mature or differentiated state. The maturity or state of differentiation may also be very important for the activity of the DC. Up to now, in vitro maturation or differentiation was triggered using cytokines and small molecules (immune response modifiers) that activate the DC's by binding to toll-like receptors (TLR's). TLR's recognize and bind small micro-organisms or microbial particles (such as bacteria and particles) which leads to activation of the DC's.

A number of immune response modifiers and (other) ligands of TLR's are known. Some of these are also used in a clinical setting. However, often, these compounds are not readily available and/or not approved for use in or in connection with human subjects.

It has now been found that some widely used vaccines, including the influenza vaccine, the BMR vaccine and the other vaccines mentioned or referred to herein, can also be used to activate DC's in vitro. Although the inventors do not wish to be bound to any specific hypothesis or explanation, some of the experimental data obtained suggests that this activation is mediated by TLR's. Also, in practice, DC's that have been activated using such vaccines are in vitro substantially comparable to DC's that have been stimulated using small molecule IRM's. They are also equally capable of producing cytokines.

Thus, such vaccines can conveniently be used to activate DC's either in vitro (for example, to differentiate DC's that have been cultivated in vitro, which can subsequently be returned to the subject from which they have been originally obtained) or directly in vivo coupled to a DC specific antibody and antigen or in situ (for example, to boost the immune system after a surgical intervention).

Dendritic cells (DCs) are the professional antigen-presenting cells of the immune system. Following infection or inflammation they undergo a complex process of maturation, and migrate to lymph nodes where they present antigens to T cells. Their decisive role in inducing immunity formed the rationale for DC immunotherapy: DCs loaded with tumor antigens are injected into cancer patients to stimulate T cells to eradicate tumors.

In the work leading up to the present invention, vaccination of cancer patients with monocyte-derived DC loaded with peptides derived from tumor-associated antigens was explored. Large amounts of clinical grade mature DC were generated according to standard, routinely implemented protocols by culturing monocytes with IL-4 and GM-CSF for 6 to 7 days. Culturing the DCs in the presence of IL-1beta, IL-6, TNF-alpha and PGE2 for 2 subsequent days induces DC maturation. HLA-A2.1+, gp100+, tyrosinase+ metastatic melanoma patients are treated with peptide-pulsed mature DC. As peptides two HLA-A2.1 restricted gp100 peptides and a tyrosinase peptide were used. All DC vaccines are co-loaded with the foreign protein KLH that serves as a control for immune competence and stimulation of a T-helper response. Vaccinations were given 3 times with 2-week intervals. It was proven that DC therapy is feasible and non-toxic, and a significant correlation between the presence of antigen specific T cells in delayed type hypersensitivity sites and clinical responses was shown. For an optimal immune response DCs should 1) effectively take up and-, process antigen, 2) mature and migrate to a neighboring lymph node and reach the area in which the T-cells reside, and 3) effectively present antigen to T-cell. If one of these steps is hampered the resulting immune response will be limited or ineffective.

To date, monocyte-derived DCs are used worldwide in clinical vaccination trials. However, it is unclear whether monocyte-derived DCs are the most optimal source of DCs for the induction of potent immune responses. It is difficult to exclude that the extensive culture period (8-9 days) and compounds required to differentiate them into DCs negatively affects DC migration.

Two major types of naturally occurring DCs can be distinguished in the blood. Both myeloid- and plasmacytoid-DCs (MDCs and PDCs) have been isolated from blood and anti-tumor responses have been reported in animal models. While blood DCs may not require extensive culture, as discussed above activation through TLRs or CD40 ligand is essential prior to re-infusion, particularly because non activated or improperly activated DC may cause T-cell tolerance rather than productive T-cell immunity.

The most commonly used method to mature ex vivo produced DC in the clinic consists of a cocktail of pro-inflammatory cytokines (IL-1beta, IL-6, TNF-alpha) and prostaglandin E2, a hormone-like structure, which is secreted upon inflammation. However, maturation of DC can be accomplished by several distinct signals that alert the resting DC to the presence of pathogens or tissue injury. Especially pathogen associated molecular patterns that activate Toll-like receptors (TLRs) have now been shown to be potent inducers of DC maturation. Recent data demonstrate that activation of DC by solely cytokines yielded DC that supported CD4+ T-cell clonal expansion, but failed to efficiently direct helper T cell differentiation. In contrast, exposure of these cells to TLR-ligands generated DC that did promote T cell help.

In the present invention, clinical applicable compounds and compositions are used that can induce maturation of blood-derived DCs (both MDC and PDC) via TLRs and thereby can induce optimally equip the DCs to exert their immunomodulatory function.

### EXAMPLE 1

### Isolation of pDC's By Positive Selection, Activation with FSME and Loading with Tumor-Derived Antigens (Peptides)

PDC are purified from peripheral blood lymphocytes by positive sorting using anti-BDCA-4 conjugated magnetic microbeads (Miltenyi Biotec GmbH, Bergisch Gladbach, Germany) according to the manufacturer's instructions. Exclusive expression of CD304 (BDCA-4/Neuropilin-1) on plasmacytoid dendritic cells allows their direct isolation. The resulting PDC-enriched preparations are consistently more than 95% pure as assessed by flow cytometry (CD123⁺/BDCA-2⁺, FIG. 1A). PDCs were adjusted to 1*10⁶ cells/ml in X-VIVO-15 (Cambrex, Verviers, Belgium) supplemented with 5% Human Serum (HS), 10 ng/ml IL-3, and 0.1 μg/ml FSME for 8 hours at 37° C. In the last 2 hours with synthetic tumor-derived peptides gp100 and tyrosinase were added. Thereafter cells were washed extensively. Analyses performed by flow cytometry revealed the expression of costimulatory molecules and peptide-loaded activated pDC's are then resuspended in physiological salt solution (0.2 ml), harvested in a syringe and injected into patients.

### EXAMPLE 2

### Generation of DC-SIGN Antibody-KLH-Vaccine Conjugates

The chemical cross-linker sulfosuccinimidyl 4-(N-maleimidomethyl)cyclohexane-1-carboxylate (sSMCC; Pierce, Rockford, Ill.) was conjugated to KLH and the vaccine FMSE according to the manufacturer's protocol. Protected sulfhydryl groups were introduced to the humanized antihuman DC-SIGN antibody hD1V1G2/G4 (hD1) with N-succinimidyl-S-acetylthiopropionate (SATP; Pierce) and were reduced with hydroxylamine hydrochloride (Pierce) using the manufacturer's protocol. Subsequently, hD1 was added to sSMCC-treated KLH and FSME in phosphate-buffered saline (PBS, pH 7.4) and allowed to react for 16 hours at 4° C. Unbound sites were alkylated by adding iodoacetamide (Sigma-Aldrich, St Louis, Mo.) to a final concentration of 25 mM, followed by 30-minute incubation at room temperature. The protein mixture was loaded onto a Superose 6 column (24-mL bed volume; Amersham Pharmacia Biotech, Uppsala, Sweden), and fractions were collected and analyzed by sodium dodecyl sulfate-polyacrylamide gel electrophoresis (SDS-PAGE). Fractions containing hD1-KLH were pooled and fractions containing free hD1 were discarded.

Binding of hD1-KLH-FSME to DCs was assessed by immunofluorescence and flow cytometry. DCs were incubated with or without 10 μg/ml hD1-KLH. After a one-hour incubation at 4° C., cells were washed and incubated with Alexa Fluor 647—labeled anti-human IgG antibody. Cells were analyzed on a FACSCalibur flow cytometer using CellQuest software (BD Biosciences, San Jose, Calif.).

### EXAMPLE 3

### Use of FMSE to Activate Plasmacytoid Dendritic Cells

This Example describes the use of the readily available FSME vaccine (clinical grade applicable) to generate clinically applicable mature pDC's under GMP conditions. The culture protocol described in this Example allows the generation of potent pDC activation in terms of phenotype and secretion of type I IFN.

For the use of pDCs as cellular vaccines in cancer immunotherapy, pDCs have to be activated and loaded with relevant tumor antigen. In addition, it was found that the pDC's obtained by the methods described herein have the ability to migrate towards draining lymph nodes and the ability to produce type I IFN, as determined by measuring the kinetics of acquisition of migratory function, cytokine production and effect on T-cell function.

Based upon phenotype and IFN-α secretion, it was found that the activation of pDC's by FSME is regulated trough TLR-9 signaling. PDCs matured by this factor up-regulate the expression levels of CD80, CD83, CD86, CCR7, MHC class I and MHC class II molecules. The elevated expression levels of CD80, CD83 and CD86 indicate that the generated pDCs are highly mature and capable of providing costimulatory signals needed for optimal T-cell activation. Upregulation of the receptor CCR7 suggests that pDCs acquire CCL21-driven chemotactic ability. It was also found that FSME vaccine activated pDCs gain migratory capacity toward CCL21, a chemokine produced in secondary lymphoid organs.

It was also found that 6 hours of stimulation with commercial FSME vaccine yielded, on highly (GMP-protocol) purified pre-pDCs, phenotypically matured pDCs comparable to the DC's that can be obtained with synthetic TLR ligands (pDCs activated with CpG-C, which were used as a positive control). This is important because it has been shown that type I IFN secretion by pDCs is highest during the first 12 hours, and that after 12 hours of stimulation pDCs tend to show a diminished secretion of type I IFN.

A specifically relevant finding is the upregulation of the expression of MHC class I and II, showing the capacity to present antigen to CD4⁺ and CD8⁺ T cells, as well as the secretion of IFN-α. pDCs become refractory to secrete type I IFN after stimulation via TLR.

All cultures were performed in triplicate and results are shown as the mean±SD. Significant difference from control according to Student's t test.

### EXAMPLE 3A

### Isolation and Activation of pDC's

pDC's were isolated under GMP conditions using the CliniMACS system (Miltenyi Biotech, Germany) and activated with FSME vaccine (invention) or the synthetic TLR ligand CpG C (positive control).

Buffy coats or apheresis material were obtained from healthy volunteers according to institutional guidelines and pDCs were purified by positive isolation using the CliniMACS system, and anti-BDCA-4-conjugated magnetic microbeads (Miltenyi Biotec) and adjusted to 10⁶ cells/ml in X-VIVO-15 (Cambrex) in 5% HS, supplemented with 10 ng/ml IL-3, 5 μg/ml CpG C or FSME vaccine (1:10).

### EXAMPLE 3B

### Determining the Phenotype of the pDC's

The phenotype of the pDC populations was determined by flow cytometry. The following primary monoclonal antibodies (mAbs) and the appropriate isotype controls were used: anti-HLA-ABC (W6/32), anti-HLA DR/DP (Q5/13) and anti-CD80 (all Becton Dickinson, Mountain View, Calif., USA); anti-CD83 (Beckman Coulter, Mijdrecht, the Netherlands), anti-CD86 (Pharmingen, San Diego, Calif., USA), anti-CCR7 (R&D Systems); followed by goat-anti-mouse PE.

It was found that stimulation of pDC with FSME vaccine led to an increased number of binding antibodies specific for CD80, CD86, CD83, MHC class I, MHC class II and CCR7 as compared to stimulation with IL-3 (see FIG. 1). The increased binding of antibodies after activation with FSME was comparable with the binding after stimulation with CpG-C.

### EXAMPLE 3C

### Cytokine Detection

Supernatants were collected from pDC cultures after 6 to 16 h of stimulation, and IFN-alpha production was analyzed with murine monoclonal capture and HRP-conjugated anti-IFN-□lpha antibodies (BenderMed systems) using standard ELISA procedures.

To analyze the T helper cell profile, supernatants were collected after 6 days of pDC-PBLs in culture, T cells were harvested, washed and resuspended to 2*10⁵/100 μl and stimulated O/N with FSME vaccine. Cytokines in the supernatant were analyzed with a cytometric bead array for human Th1/Th2 cytokines (BD Biosciences, San Diego, Calif.) according to the manufacturer's protocol (detecting IL-2, IL-4, IL-5, IL-10, IFN-gamma and TNF-alpha).

It was found that stimulation of pDC with FSME vaccine led to an increased production of IFN-alpha (see FIG. 1). The increased production of this cytokine after activation with FSME was comparable with the production after stimulation with CpG-C.

T cells (both allogeneic as well as autologous) cocultured with FSME-stimulated pDC's were equally efficient as CpG-C stimulated pDC's in producing cytokines. High levels of IFN-gamma, TNF-alpha and IL-2 were measured indicating full T cell activation.

### EXAMPLE 3D

### Chemotaxis

For CCR7-mediated migration a standard in vitro transwell migration assay was performed. 5 μm pore size polycarbonate membranes (Costar, London, UK) were placed upon an aliquot of 600 μl X-Vivo 15 medium with 5% HS with or without CCL21 (100 ng/ml; Tebu-Bio). A total of 1×10⁵ pDC in 100 μl culture medium were seeded in the upper compartment. To analyze migration toward the gradient, CCL21 was added to the lower wells. Spontaneous migration and kinesis were measured by incubation of the cells in a transwell without CCL21 in the lower well. pDC were allowed to migrate for 120 min. in a 5% CO₂, humidified incubator at 37° C. After incubation, beads (Beckman Coulter) were added to 600 microliter culture medium containing migrated pDC and then counted by flow cytometry. A total amount of 5000 beads were counted and correlated to amount of DC measured. All conditions were tested in duplicate.

It was found (see FIG. 3) that pDC's activated with FSME show a migratory capacity which is similar to the migratory capacity of pDC's that had been activated with the synthetic TLR-ligands R848 and CpG-C (which pDC's were used as positive controls).

### EXAMPLE 3E

### Measuring the Mixed Lymphocyte Reaction

The allostimulatory capacity of the pDC was tested in a mixed lymphocyte reaction (MLR). Allogeneic T cells were co-cultured with differently matured pDCs in a 96-well round bottom plate (pDC:T cell ratio 1:20 with 1*10⁵ PBL). After 6 days of culture, 1 μCi/well of tritiated thymidine was added for 16 h and incorporation was measured in a beta-counter.

It was found that coculturing of FSME-stimulated pDC's led to an increased proliferation of allogeneic T cells as compared to IL-3 stimulated pDC's. The increased proliferation after activation with FSME was comparable with pDC-induced T cell proliferation after activation with CpG-C.

### EXAMPLE 3F

### Measuring Specific KLH Responses

Cellular responses against the protein keyhole limpet hemocyanin (KLH) were measured in a proliferation assay. In our vaccination studies, KLH is added to immature DC culture as an immunomonitoring tool. Peripheral blood mononuclear cells (PBMC) were isolated from blood samples from four patients taken after four biweekly vaccinations with mature DC. CD4⁺ T cells were isolated with a CD4⁺ T cell isolation kit (Miltenyi Biotec, Bergisch Gladbach, Germany) according to the manufacturer's instructions. The purified T cells were plated in a 96-well tissue culture microplate with autologous pDCs that were cultured with or without KLH and matured with CpG-C or FSME. After 4 days of culture, 1 μCi/well of tritiated thymidine was added for 16 h and incorporation was measured in a beta-counter.

It was found that coculturing of FSME-stimulated KLH-loaded pDC's led to an increased proliferation of autologous T cells as compared to IL-3 stimulated KLH-loaded pDC's. The increased proliferation after activation with FSME was comparable with pDC-induced T cell proliferation after activation with CpG-C.

### EXAMPLE 4

### Use of Vaccines to Activate Monocyte-Derived Dendritic Cells

This example shows that mDC's can be activated using commercially available vaccines, and shows that preferably, a combination or mixture of vaccines is used to activate mDC's.

### EXAMPLE 4A

### Antibodies and Immunostaining

The phenotype of the DC populations was determined by flow cytometry. The following primary monoclonal antibodies (mAbs) or the appropriate isotype controls were used: anti HLA-ABC (W6/32), anti-HLA DR/DP (Q5/13) and anti-CD80 (all Becton Dickinson, Mountain View, Calif., USA), anti-CD83 (Beckman Coulter, Mijdrecht, the Netherlands), anti-CD86 (Pharmingen, San Diego, Calif., USA), anti-CCR7 (R&D systems), anti-CD14 (Beckman Coulter), followed by Alexa Fluor 488 conjugated goat anti-mouse IgG (Molecular Probes).

It was found (see FIG. 4) that stimulation of monocyte-derived DC with BCG, Typhim vaccines led to a slightly increased number of binding antibodies specific for CD80, CD86, CD83, MHC class I, and MHC class II as compared to no stimulation. However, the combination of vaccines either BCG, Typhim, and Influvac or BCG, Typhim and Act-HIB led to high CD80, CD86, CD83, MHC class I, and MHC class II (see FIG. 5). The expression of these molecules was comparable to the expression after maturation with a cocktail of cytokines (IL-1beta, TNF-alpha, IL-6 and PGE2) or after TLR mediated maturation (poly I:C and R848). Addition of PGE2 to the combination of vaccines resulted in an upregulation of CCR7 to levels comparable to the cytokine-matured DC (see FIG. 5).

### EXAMPLE 4B

### Culture Media and Cytokines

For DC culture, X-VIVO 15 (BioWhittaker, Walkersville, Md., USA) was supplemented with 2% human serum (HS; serum of six blood donors type AB was pooled; Sanquin, Bloodbank Zuid-Oost, Nijmegen, the Netherlands), IL-4 (300 U/ml) and GM-CSF (450 U/ml) (both from Strathmann, Hamburg, Germany). For DC maturation the following products were used: recombinant TNFα (100 ng/ml; CellGenix, Freiburg, Germany), IL-1β (5 ng/ml; Immunotools, Friesoythe, Germany), PGE₂ (10 μg/ml; Pharmacia & Upjohn, Puurs, Belgium), IL-6 (15 ng/ml; CellGenix) and for the vaccine matured DC: BCG (4%), Typhim (4%), and Influvac (4%) (vaccineA-DC); BCG (4%), Typhim (4%) and Act-HIB (4%) (vaccineB-DC); BCG (4%), Typhim (4%), Influvac (4%) and 10 pg/ml PGE₂ (vaccineA-PGE2-DC); BCG (4%), Typhim (4%), Act-HIB (4%) and 10 μg/ml PGE₂ (vaccineB-PGE2-DC).

### EXAMPLE 4C

### Vaccines Used

Act-HIB® (Aventis Pasteur, Brussels, Belgium), BCG vaccin SSI (Nederlands Vaccin Instituut, Bilthoven, The Netherlands), BMR vaccine (Bof-Mazelen-, Rubellavaccin, Nederlands Vaccin Instituut, Bilthoven, The Netherlands), FSME-IMMUN (Baxter AG, Vienna, Austria), Infanrix-IPV+HIB (GlaxoSmithKline BV, Zeist, The Netherlands), Influvac 2007/2008 (Solvay Pharmaceuticals, Weesp, The Netherlands), Inactivated Rabies vaccine Merieux HDCV (Sanofi Pasteur MSD, Brussels, Belgium), Typhim Vi (Sanofi Pasteur MSD, Brussels, Belgium).

### EXAMPLE 4D

### Preparation of mDC's From Peripheral Blood Precursors

DC were generated from PBMC prepared from leukapheresis products or from buffy coats essentially as described previously. Buffy coats were obtained from healthy volunteers according to institutional guidelines. Plastic-adherent monocytes from leukapheresis or buffy coats were cultured in X-VIVO 15™ medium (BioWhittaker, Walkersville, Md.) supplemented with 2% pooled human serum (HS) (Bloodbank Rivierenland, Nijmegen, The Netherlands), IL-4 (500 U/ml) and GM-CSF (800 U/ml) (both from CellGenix, Freiburg, Germany). On day 6 or 7 cells were either kept in the immature state or one of the following maturation cocktail was added for 48 h: autologous MCM (30%, v/v) and 10 ng/ml recombinant TNF-α (CellGenix) and 10 μg/ml PGE₂ (Pharmacia & Upjohn, Puurs, Belgium) or 10 ng/ml recombinant TNF-α (CellGenix), 5 ng/ml IL-1β (ImmunoTools, Friesoythe, Germany), 15 ng/ml IL-6 (CellGenix) and 10 μg/ml PGE₂ (Pharmacia) (conventional DC, cDC); 20 μg/ml poly(I:C) and 3 μg/ml R848 (TLR-DC); 20 μg/ml poly(I:C), 3 μg/ml R848 and 10 μg/ml PGE₂ (TLR-PGE2-DC); BCG (4%), Typhim (4%), and Influvac (4%) (vaccineA-DC); BCG (4%), Typhim (4%) and Act-HIB (4%) (vaccineB-DC); BCG (4%), Typhim (4%), Influvac (4%) and 10 μg/ml PGE₂ (vaccineA-PGE2-DC); BCG (4%), Typhim (4%), Act-HIB (4%) and 10 μg/ml PGE₂ (vaccineB-PGE2-DC). Single vaccines were added at a concentration of 5%.

### EXAMPLE 4E

### TLR Ligand Screening

The presence of TLR ligands was tested on recombinant HEK-293 cell lines that functionally express a given TLR protein as well as a reporter gene driven by a NFKB inducible promoter. TLR ligand screening was performed by InvivoGen (InvivoGen Europe, Toulouse, France).

### EXAMPLE 4F

### In Vitro Migration Assays

For random migration on fibronectin, flat-bottomed plates 96-well plates (Costar, Corning, N.Y.) were coated with 20 μg/ml fibronectin (Roche, Mannheim, Germany) for 60 min at 37° C. and blocked with 0.01% gelatin (Sigma Chemical Co., St. Louis, Mo.) for 30 min at 37° C. 4000 DC per well were seeded on fibronectin-coated plates and recorded for 60 min at 37° C., after which migration tracks of individual DC were analyzed using an automated cell tracking system. The migrated distance is the traversed path in 60 min.

For CCR7-mediated migration a standard in vitro transwell migration assay was used. Transwell inserts with 5 μm pore size polycarbonate membranes (Costar, London, UK) were preincubated with 100 μl of X-Vivo 15™/2% HS in 24-well plates, each well containing 600 pl of the same medium. A total of 1×10⁵ DC were seeded in the upper compartment. To analyze migration toward the gradient, CCL21 (100 ng/ml) was added to the lower wells. Spontaneous migration and kinesis were measured by incubation of the cells in a transwell without or with CCL21 in both the upper and the lower well, respectively. DC were allowed to migrate for 60 min. in a 5% CO₂, humidified incubator at 37° C. After this time period, DC were harvested from the lower chamber and counted by flow cytometry. All conditions were tested in duplicate. The results are shown in FIG. 6.

### EXAMPLE 4G

### CD40L Stimulation

DC were harvested, washed and seeded in a 96-well roundbottomed plate at 50×10³ cells in 100 μl per well. To mimic the interaction with CD40L-expressing Th-cells, CD40L trimers (Leinco Technologies, Mo., USA) were added to the vaccine-matured DC at a concentration of 1 μg/ml. Twenty-four hour supernatants were analyzed by IL-12p70 ELISA.

### EXAMPLE 4H

### Production of IL-12p70 by Activated mDC's

The production of IL-12p70 was measured in the supernatants 48 hr after induction of maturation or 24 hr after secondary stimulation with CD40L using a standard sandwich ELISA (Pierce Biotechnology, Rockford). The procedure was performed according to the manufacturer's instructions. The results are shown in FIG. 5.

### EXAMPLE 4I

### Mixed Lymphocyte Reaction (MLR)

The ability of the DC to induce T cell proliferation was studied in an allogeneic proliferation assay. Briefly, DC were added to 1×10⁵ freshly isolated allogeneic non-adherent PBMC from a healthy donor. After 4 days of culture, 1 μCi of tritiated thymidine was added per well. Incorporation of tritiated thymidine was measured in a beta-counter after 8 hours of pulsing. Cytokine production was measured in all MLR-supernatants after 48 hours by cytometric bead array (Th1/Th2 Cytokine CBA 1; BD PharMingen, San Diego, Calif.).

### EXAMPLE 4J

### Antigen-Specific Proliferation Assay

Cellular responses against the protein keyhole limpet hemocyanin (KLH) were measured in a proliferation assay. KLH is added to the immature DC culture as a immunomonitoring tool. Peripheral blood mononuclear cells (PBMC) were isolated from blood sample from four patients taken after four biweekly vaccinations with mature DC. CD4+ T cells were isolated with a CD4+ T cell isolation kit (Miltenyi Biotech, Bergisch Gladbach, Germany) according to the manufacturer's instructions. The purified T cells were plated in a 96-well tissue culture microplate with autologous DC that were cultured with or without KLH and matured with the cytokine cocktail, with poly(I:C) and R848, or with vaccines with or without PGE2. After 4 days of culture, 1 μCi/well of tritiated thymidine was added for 8 h, and incorporation of tritiated thymidine was measured in a beta-counter. Cytokine production was measured in the supernatants after 24/48 hours by cytometric bead array (Th1 /Th2 Cytokine CBA 1, BD PharMingen, San Diego, Calif.). The results are shown in FIG. 6.

### EXAMPLE 5

### Production of Vaccine-Matured Antigen-Loaded mDC's

In this Example, vaccine matured mDC's were loaded with the tumor antigen gp100. Loading of the antigen was performed by electroporation of the DC's with mRNA coding for gp100. This example shows that vaccine matured DC's can be antigen-loaded by using mRNA encoding the antigen instead of the antigen itself (i.e. in the form of tumor lysates, tumor protein or defined tumor peptides).

mDC's activated with cytokines, mDCs activated with synthetic TLR-ligands, and mDC's activated with vaccines were electroporated with gp100 mRNA and protein expression was analyzed using FACS analysis and on cytospins 2 hr after electroporation.

Mature DCs were washed twice in PBS and once in OptiMEM® without phenol red (Invitrogen, Breda, The Netherlands). 20 μg RNA (gp100 RNA, Curevac) was transferred to a 4-mm cuvette (BioRad, Veenendaal, The Netherlands) and 10×10⁶ DC were added in 200 μl OptiMEM® and incubated for 3 min before being pulsed with an exponential decay pulse at 300 V and 150 μF in a Genepulser Xcell (BioRad) according to a standard protocol (see for example Beekman et al, manuscript submitted for publication). Immediately after electroporation the cells were transferred to warm (37° C.) X-VIVO 15™ without phenol red (Cambrex Bio Science, Verviers, Belgium) supplemented with 5% HS and left for at least 2 h at 37° C., before further manipulations were performed. Electroporation efficiency was analyzed by intracellular staining and FACS analysis.

### EXAMPLE 6

### Testing of Different Vaccines for Their Ability to Interact with TLR's and to Activate DC's

### EXAMPLE 6A

### Ability to Interact with TLR's Expressed by mDC's or pDC's

The vaccines listed in Table 4 were tested for their capacity to interact with TLRs. HEK293 cells stably transfected with plasmids constitutively expressing human TLR genes were used to investigate the chosen vaccines. The HEK293 cell line was selected for its null or low basal expression of the TLR genes. As shown in Table 4, components of 8 vaccines were able to activate TLR-expressing HEK293 transfectants. TLR2-mediated activation was observed with BCG and Infanrix, the latter and Act-Hib were also able to activate via TLR4. BMR, a vaccine composed of vaccination against measles, mumps and rubella was able to activate via TLR5 and TLR9. TLR9-mediated activation was also observed with the FSME, Act-Hib and Rabies vaccines.

TABLE 4
testing of different vaccines for their ability to
interact with different TLR's expressed by DC's. (*)

| Vaccine | TLR2 | TLR4 | TLR5 | TLR7 | TLR8 | TLR9 |
|---|---|---|---|---|---|---|
| TYPHIM Vi | | | | | | |
| BCG | + | | | | | +? |
| ACT-HIB | | + | | | | + |
| FSME | | | | | | + |
| Rabies | | | | | | + |
| BMR | | | + | | | +? |
| INFANRIX | + | + | | | | +? |
| INFLUVAC | | | | | | |

Note:
“+?” indicates that the vaccine is expected to bind to said TLR, but that this has not yet been experimentally demonstrated. As further described herein TLR-2, TLR-4 and TLR-5 are predominantly expressed by mDC's, and TLR-7 and TLR-9 are predominantly expressed by pDC's.

### EXAMPLE 6B

### Use of Preventive Vaccines to Induce DC Maturation (Via TLR Activation)

The vaccines listed in Table 4 were tested for their ability to induce DC maturation in vitro. The vaccines were added at 5% (v/v) concentration to the culture medium. The majority of the vaccines were non-toxic and yielded normal numbers of DC in the concentrations used (data not shown). Activation of the DC's was determined using one or more of the assays described in Example 3 (for pDC's) or Example 4 (for pDC's). The results are shown in FIG. 7 (phenotype) and FIG. 8 (IFN-alpha production).

For the activation of pDC's, it was found that four vaccines (FSME, INFANRIX, BMR, Rabies) had the ability to induce IFN-α production, whereas FSME and to a lesser extend Act-Hib and BCG had the ability to induce the differentiation of pre-pDCs into pDCs (FIG. 7).

This shows that vaccines are able to stimulate pDCs and that different vaccines may be used to exert different effects on pDCs: Some vaccines may be used to induce high levels of type I IFNs without having a major activity with respect to inducing antigen presenting molecules. Other vaccines (such as Act-Hib) can be used to upregulate the expression of costimulatory molecules CD80 and CD86 without resulting in a major increase in the production of type I IFN. It is also possible that the use of a combination of two or more of these vaccines could lead to induction of both IFN-α production as well as phenotypic maturation of pDCs.

Other vaccines (such as, in particular, FSME) were found to be able to induce both IFN-α production and phenotypic maturation of pDCs.

To confirm that endosomal maturation and binding of the vaccine components to TLR's are involved in the vaccine-induced maturation of pDC's according to the invention, pDCs were activated with the vaccines in the presence or absence of chloroquine. It was found that treatment with chloroquine completely inhibited the IFN-α secretion and differentiation of vaccine-activated pDCs. (see FIG. 2). This suggest that, in particular for pDC's, the effects induced by the vaccines used in the invention are likely dependent on endosomal maturation and binding of the vaccine components to TLR's (similar to what has been reported for the synthetic TLR targeting compounds like CpG and R848).

The vaccines listed in Table 4 that can interact with TLR-2, TLR-4 and/or TLR-5 were most suited for activating mDC's, whereas the vaccines listed in Table 4 that can interact with TLR-7, TLR-8 and/or especially TLR-9 were most suited for activating pDC's (detailed data not shown).

### EXAMPLE 6C

### Use of Combinations of Vaccines to Mature mDC's

Vaccines with different TLR ligands (and each with the ability to individually induce at least some maturation of DC's) were combined and tested for their ability to mature mDC's. As shown in FIG. 1, expression of maturation markers was strongly increased on DC matured with a combination of BCG, Typhim and Influvac, to levels that were comparable to those obtained for cytokine-matured DC (positive control). As shown in FIG. 2, compared to DC treated with single vaccines, such as BCG or Typhim, IL-12p70 production of the vaccine-matured mDC's was strongly increased, suggesting a synergistic effect of the vaccine combination compared to the corresponding separate vaccines

It was also found that, after maturation with the vaccine combination, expression of the chemokine receptor CCR7 (involved in DC migration to T cell areas of the lymph nodes) by the vaccine-matured mDC's was slightly increased; and could be increased further by addition of PGE₂ to a level that was comparable to that obtained with cytokine-matured DC (positive control). In addition, adding PGE2 improved the ability of the vaccine-matured DC's to migrate (i.e. towards lymph nodes), as determined using the random migration assay and CCR7 mediated migration assay described in Example 4. The results are shown in FIGS. 3A and 3B.

The vaccine-matured DC were also tested for their ability to stimulate antigen-specific T cells, by measuring KLH-specific proliferation of CD4+ T cells isolated from patients that had been vaccinated previously with KLH-loaded DC. The results are shown in FIG. 4.

### EXAMPLE 7

### Use of Vaccine-Activated DC's in Cancer Immunotherapy

From the further disclosure and results presented herein, it can be seen that vaccine-matured mDC's, loaded with peptides against gp100 and tyrosinase and KLH, can migrate into the T-cell area of lymph nodes in vivo and are capable of eliciting antigen specific T- and B-cell responses.

It can also be seen that pDC are at least equally strong inducers of immune responses when compared with their myeloid counterparts (mDC's), and can efficiently promote both Th2 as well as Th1 responses and produce high amounts of IFNalpha and IL12 when properly activated with the vaccines used herein.

For testing in human volunteers, after obtaining all necessary approvals and permissions, 5 patients are vaccinated with PDC, cultured under GMP conditions. Peripheral blood mononuclear cells are obtained by leukapheresis. From these cells pDC are isolated by magnetic cell sorting with clinical grade antibodies against BDCA-4 (Miltenyi Biotec) coupled to magnetic beads. HLA-A2.1 and/or HLA-A3 and/or HLA-DR4 positive stage IV melanoma patients are administered escalating doses of 0.3×10⁶, 1×10⁶ and 3×10⁶ PDC stimulated for 6 hours with FSME vaccine pulsed with synthetic peptides derived from melanoma associated antigens gp100 and tyrosinase.

The antigen-loading of the pDC's is performed as follows: pDC are pulsed with peptides in XVivo medium at 370 C for 2 h, after which PDC are washed in PBS/autologous serum. The following peptides-(all GMP grade) will be used:

TABLE 5
Anti-tumor peptides

| | | | SEQ ID |
|---|---|---|---|
| Name | Peptide | Sequence | NO: |
| HLA-A0201 | gp100-derived | YLEPGPVTA | 1 |
| | peptide: 280-288 | | |
| HLA-A0201 | gp100-derived | KTWGQYWQV | 2 |
| | peptide: 154-162 | | |
| HLA-A0201 | tyrosinase-derived | YMDGTMSQV | 3 |
| | peptide: 369-377 | | |
| HLA-DR4 | gp100-derived | WWRQLYPEWTEA | 4 |
| | peptide: 44-59 | QRLD | |
| HLA-DR4 | tyrosinase-derived | DYSYLQDSDPDSF | 5 |
| | peptide: 448-462 | QD | |
| HLA-A3 | gp100-derived | ALLAVGATK | 6 |
| | peptide: 17-25 | | |
| HLA-A3 | MAGE-1 derived | SLFRAVITK | 7 |
| | peptides: 96-104 | | |

Isolation, culture, stimulation and pulsing of pDC will be carried out under suitable GMP/GLP conditions.

The vaccine is injected intranodally under ultrasound guidance following standard protocol (or alternatively, the vaccine will be administred i.v./i.d.) Patients are administered 3 vaccinations with a 2-week interval. One week after the last vaccination a DTH test is performed. From positive induration sites biopsies are taken for T-cell culture, immunohistochemistry and in situ tetramer staining.

Toxicity is assessed after each vaccination according to the NCI common toxicity criteria. For immunomonitoring the induction of gp100 and tyrosinase specific T cell responses in peripheral blood, DTH reaction sites and (if available) tumor material is determined. Also, cytokine profiles of responding T cells are determined. For this, before start of therapy and after each immunization peripheral blood mononuclear cells are obtained from the patient for monitoring purposes; and after three vaccinations a DTH test are performed and biopsies are taken from positive induration sites. All studies and assays are performed according to standard clinical protocols.

Tetramer analyses of PBMC for gp100 and tyrosinase are performed after the third vaccination by flow cytometry.

In all patients a DTH skin test is performed according to standard protocols in the skin of the back 1-2 weeks after the 3rd immunization with pDC's with peptide-pulsed pDC's, with KLH-pulsed pDC's. 48-hours later by 6 mm punch biopsies from each positive DTH reaction (defined as an induration of at least 2 mm in diameter) are taken. These biopsies are be split in three 2-mm portions, which are used for immunohistology, PCR analysis and T cell responses. If applicable, biopsies are taken from (sub-)cutaneous metastases.

Characterization of leukocyte infiltrates is performed with antibodies against DC markers and surface markers on infiltrating mononuclear cells. Antibodies recognizing the following antigens are employed on 4 μm frozen sections. Determination of the following markers is of particular interest:
Monocyte, macrophage, DC lineage markers: CD1a, CD11c, CD 14, CD 123, CD68, CD83, Il-3R, DC-SIGN;
Lymphocyte (activation/maturation) markers: CD3, CD4, CD8, CD28, CD45 RA/RO, CD69, Il-2 receptor, FASL-HLA and co-stimulatory molecules: CD80 (B7.1), CD86 (B7.2), CD40, HLA-class I and II, CD8;
Chemokines and chemokine receptors: immature DC predominantly express MIP-3 alpha but not MIP3 beta, whereas they express both DC-CK1. Also marked differences have been found in chemokine receptor expression: immature DC express CCR1, CCR3, CCR5, CCR6 (receptor for MIP3 alpha), whereas mature DC express predominantly CCR7 (receptor for MIP3 beta) and CXCR4. of 10, 11, 12

T cell responses will be determined as follows. For determining the proliferation and cytotoxicity of T cells, bulk cultures of T cells isolated from DTH biopsies and tumor metastases (if available) are be grown (low dose IL-2) in vitro and restimulated with peptide (gp100/tyrosinase). After one week, their proliferative capacity as well as their cytotoxic activity against peptide/protein loaded target cells and tumor cells are tested in a ³H-thymidine incorporation test and 51Cr release assay respectively. IFN-gamma and TNF-alpha release as a marker for activation are determined using Elispot assays.

Cytokines produced by the T-cells are measured using a flowcytometric assay in which IL-2, IL-4, IL-5, IL-10, IFN-gamma, TNF-alpha are determined simultaneously (Beckton & Dickinson. The same assay can be used to determine cytokines secreted by T cells from DTH and tumor biopsies after antigen specific restimulation).

Finally, tetramers (gp100, tyrosinase) will be used to identify antigen specific T cells.

1. An in vitro or ex vivo method for providing a composition that comprises at least one activated dendritic cell, which method at least comprises the steps of: a) providing a composition that comprises at least one dendritic cell, in which said composition comprises at least one plasmacytoid-derived dendritic cell and/or at least one myeloid-derived dendritic cell; b) activating said dendritic cell by contacting it with a vaccine.

2. Method according to claim 1, in which the at least one dendritic cell is brought into a state in which it is capable of stimulating T-cells and/or a T-cell mediated response.

3. Method according to claim 1, in which the vaccine comprises a formulation or preparation of one or more antigenic components that are capable of activating one or more plasmacytoid-derived dendritic cells and/or one or more myeloid-derived dendritic cells through the interaction with one or more dsRNA sensors and/or toll-like receptors (TLR's) that are expressed by the dendritic cells to be activated.

4. Method according to claim 3, in which the dendritic cells are plasmacytoid-derived dendritic cells, and in which the one or more antigenic components are capable of activating plasmacytoid-derived dendritic cells by interaction with one or more of the following TLR's expressed by the plasmacytoid-derived dendritic cells: TLR-7, TLR-8, and/or TLR-9.

5. Method according to claim 3, in which the vaccine comprises one or more of the following antigenic components: inactivated, weakened or attenuated bacteria or viruses; inactivated, weakened or attenuated viral particles; DNA, single stranded RNA or double stranded RNA that is contained in or encoded by bacteria or viruses; or any other suitable antigenic components that are based on, and/or that have been derived from, micro-organisms, such as bacterial or viral proteins, as well as cell fragments or cell fractions that have been derived from bacteria, viruses or other suitable microorganisms.

6. A plasmacytoid-derived dendritic cell and/or myeloid-derived dendritic cell that has been activated using a method as defined in any of claims 1.

7. Method according to claim 1; wherein in a further step c) said dendritic cell is loaded with the one or more desired antigens.

8. Method according to claim 7, in which, in step c), the one or more desired antigens is one or more tumour-associated antigens.

9. Method according to claim 7, for providing one or more tolerogenic dendritic cells.

10. Method according to claim 9, for providing one or more tolerogenic dendritic cells for the prevention and/or treatment of an auto-immune disease, of an inflammatory disease or disorder such as rheumatoid arthritis or asthma, of a transplant rejection and/or of an allergy in a subject.

11. Method for providing at least one plasmacytoid-derived dendritic cell and/or at least one tolerogenic myeloid-derived dendritic cell for use in immunotherapy in a subject, which method at least comprises the steps of: a) harvesting a sample or population of said cells from said subject; b) activating these cells in said sample or population using a vaccine; and c) loading these cells with one or more antigens that are suitable for immunotherapy in said subject.

12. Method according to claim 11, wherein said activated dendritic cells is loaded with one or more tumor-associated antigens.

13. Method for immunotherapy in a subject, which method at least comprises the steps of: a) harvesting a sample or population of plasmacytoid-derived dendritic cell and/or myeloid-derived dendritic cell from said subject; b) activating these cells in said sample or population using a vaccine; c} loading these cells with one or more antigens that are suitable for immunotherapy in said subject; and d) administering the activated and loaded dendritic cells to said subject.

14. Method according to claim 13, for immunotherapy of cancer in a subject, in which, in step c), the activated dendritic cell is loaded with one or more tumor-associated antigens; and/or with one or more suitable (synthetic or semi-synthetic) tumour-specific peptide antigens; and/or with a cell lysate, cell fraction, cell fragment or cell extract that has been obtained from a tumor cell or tumor cell line.

15. A part for providing activated dendritic cells as defined in claim 1 that have been loaded with one or more desired antigens, at least comprising a vaccine for activating the cells and the one or more desired antigens.
